(19) United States
(12) Patent Application Publication
(10) Pub. No.: US 2021/0310075 A1
(43) Pub. Date: Oct. 7, 2021

(54) Cancer Classification with Synthetic Training Samples

(71) Applicant: Grail, Inc., Menlo Park, CA (US)

(72) Inventors: M. Cyrus Maher, San Mateo, CA (US); Samuel S. Gross, Sunnyvale, CA (US); Joshua Newman, Mountain View, CA (US); Joerg Bredno, Menlo Park, CA (US); Ognjen Nikolic, Menlo Park, CA (US)

(21) Appl. No.: 17/216,555

(22) Filed: Mar. 29, 2021

Related U.S. Application Data
(60) Provisional application No. 63/154,667, filed on Feb. 27, 2021.
(60) Provisional application No. 63/001,729, filed on Mar. 30, 2020.

(51) Int. Cl. C12Q 1/6886; G16B 40/20

(52) U.S. Cl. CPC C12Q 1/6886; C12Q 2600/154; G16B 40/20

(57) ABSTRACT

Methods and systems for detecting cancer and/or determining a cancer tissue of origin are disclosed. A multiclass cancer classifier is disclosed that is trained with a plurality of biological samples containing cfDNA fragments and at least one synthetic training sample generated from the biological samples. The analytics system generates the synthetic training sample by sampling fragments from a training sample labeled as cancer and sampling fragments from another training sample labeled as non-cancer. The sampling probability is determined based on a limit of detection of the cancer classifier, e.g., in order to generate synthetic training samples with cancer tumor fraction proximate to the limit of detection.

### RELATED APPLICATIONS

This application claims the benefit of and priority to U.S. Provisional Application No. 63/001,729 filed Mar. 30, 2020, and U.S. Provisional Application No. 63/154,667 filed Feb. 27, 2021, both of which are incorporated by reference in their entireties. All other publications, patents, and patent applications described herein are incorporated by reference in their entireties. In the event of a conflict between a term herein and a term in an incorporated reference, the term herein controls.

### BACKGROUND

### Field of Art

Deoxyribonucleic acid (DNA) methylation plays an important role in regulating gene expression. Aberrant DNA methylation has been implicated in many disease processes, including cancer. DNA methylation profiling using methylation sequencing (e.g., whole genome bisulfite sequencing (WGBS)) is increasingly recognized as a valuable diagnostic tool for detection, diagnosis, and/or monitoring of cancer. For example, specific patterns of differentially methylated regions and/or allele specific methylation patterns may be useful as molecular markers for non-invasive diagnostics using circulating cell-free (cf) DNA. However, there remains a need in the art for improved methods for analyzing methylation sequencing data from cell-free DNA for the detection, diagnosis, and/or monitoring of diseases, such as cancer.

The present disclosure is directed to addressing one or more of these above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

Early detection of a disease state (such as cancer) in subjects is important as it allows for earlier treatment and therefore a greater chance for survival. Sequencing of DNA fragments in cell-free (cf) DNA sample can be used to identify features that can be used for disease classification. For example, in cancer assessment, cell-free DNA based features (such as presence or absence of somatic variant, methylation status, or other genetic aberrations) from a blood sample can provide insight into whether a subject may have cancer, and further insight on what type of cancer the subject may have. Towards that end, this description includes systems and methods for analyzing cell-free DNA sequencing data for determining a subject's likelihood of having a disease.

The present disclosure addresses the problems identified above by providing improved systems and methods for making use of existing data in order to improve the performance of classifiers that discriminate disease conditions. Generally, the disclosed systems and methods accomplish this by enabling synthetic expansion of biological data sets, particularly those data sets based on genomic data extracted from nucleic acid sequencing of biological samples, e.g., tumor samples, liquid biopsies, etc. In some embodiments, this is accomplished by generating augmented data constructs that are based upon one or more data constructs generated from a biological sample. The augmented data constructs can be used to supplement existing data constructs generated directly from biological samples, generating an expanded biological data set. These expanded biological data sets can facilitate the training of disease classifiers with higher specificity and/or sensitivity than disease classifiers trained against only the original data constructs generated directly from the biological samples. The improvement can be attributable to several factors. For example, by using a larger (expanded) training data set, the incidence of data overfitting is reduced because the classifier can better generalize trends in the data. In addition, by controlling the amount of disease signal in an augmented data construct, the expanded data set can be constructed such that it contains a higher percentage of data constructs having disease signals near the level of detection (LOD) of the classifier. This, in turn, can allow better training of the model in feature space where the disease signal is scarcer.

An analytics system processes a multitude of sequencing data from a plurality of samples (e.g., a plurality of cancer and non-cancer samples) to identify features that are subsequently utilized for cancer classification. The analytics system generates at least one synthetic training sample from the obtained biological samples. The analytics system generates the synthetic training sample by sampling fragments from a training sample labeled as cancer and sampling fragments from another training sample labeled as non-cancer. The analytics system may further label the synthetic training sample with a particular cancer type belonging to the cancer training sample used to generate the synthetic training sample. The sampling probability is determined based on a limit of detection of the cancer classifier, e.g., in order to generate synthetic training samples with cancer tumor fraction proximate to the limit of detection. With the sequencing data, the analytics system is able to train and deploy a cancer classifier for generating a cancer prediction for a test sample.

In selecting which training samples are used to train the cancer classifier, the analytics uses training samples that have already been identified and labeled as having one or a number of cancer types, as well as training samples that are from healthy individuals that are labeled as non-cancer. Each training sample includes a set of fragments. For each training sample, the analytics system generates a feature vector, for example, by assigning a score to each of the identified features. The analytics system may group the training samples into sets of one or more training samples for iterative training of the cancer classifier. The analytics system inputs each set of feature vectors into the cancer classifier and adjusts classification parameters in the cancer classifier such that a function of the cancer classifier calculates cancer predictions that predict the labels of the training samples in the set based on the feature vectors and the classification parameters with an above-threshold accuracy. The cancer classifier is iteratively trained by iterating the above steps through each set of training samples.

During deployment, the analytics system generates a feature vector for a test sample in a similar manner to the training samples, e.g., by assigning a score to each of a plurality of features in a feature vector for each of the test samples. Then the analytics system inputs the feature vector for the test sample into the cancer classifier which returns a cancer prediction. In one embodiment, the cancer classifier may be configured as a binary classifier to return a cancer prediction of a likelihood of having or not having cancer. In another embodiment, the cancer classifier may be configured as a multiclass classifier to return a cancer prediction with prediction values for each of a plurality of cancer types.

### BRIEF DESCRIPTION OF DRAWINGS

FIG. 1A is an exemplary flowchart describing a process of sequencing a fragment of cell-free (cf) DNA to obtain a methylation state vector, according to one or more embodiments.

FIG. 1B is an exemplary illustration of the process of FIG. 1A of sequencing a fragment of cell-free (cf) DNA to obtain a methylation state vector, according to one or more embodiments.

FIGS. 2A & 2B exemplary illustrate flowcharts describing a process of determining anomalously methylated fragments from a sample, according to one or more embodiments.

FIG. 3 illustrates an exemplary process of generating a synthetic training sample, according to one or more embodiments.

FIG. 4 is an exemplary flowchart describing a process of generating a synthetic training sample for training of a cancer classifier, according to one or more embodiments.

FIG. 5A illustrates an example workflow 500 for generating augmented data and optionally training a classifier to discriminate disease states from one another, according to one or more embodiments.

FIG. 5B illustrates an example workflow for generating supplemental data, according to one or more embodiments.

FIG. 6A is an exemplary flowchart describing a process of training a cancer classifier, according to one or more embodiments.

FIG. 6B illustrates an example generation of feature vectors used for training the cancer classifier, according to one or more embodiments.

FIG. 7A illustrates an exemplary flowchart of devices for sequencing nucleic acid samples according to one or more embodiments.

FIG. 7B is an exemplary block diagram of an analytics system, according to one or more embodiments.

FIG. 8 illustrates exemplary graphs showing cancer prediction accuracy of a multiclass cancer classifier for various cancer types, according to an example implementation.

FIG. 9 illustrates exemplary graphs showing cancer prediction accuracy of a multiclass cancer classifier for various cancer types after first using a binary cancer classifier, according to an example implementation.

FIG. 10 illustrates an exemplary confusion matrix demonstrating performance of a trained cancer classifier, according to an example implementation.

FIG. 11 illustrates an exemplary table comparing performance of a cancer classifier trained with synthetic training samples, according to some example implementations.

FIGS. 12A-12C illustrate example cancer probability graphs, according to one or more embodiments.

FIG. 13 illustrates the evaluation of two classifiers trained to detect cancer based on genomic characteristics of cell-free DNA in patient samples, according to one or more embodiments.

FIG. 14 illustrates an exemplary graph of training set sensitivity vs. test set sensitivity, according to one or more embodiments.

FIG. 15 illustrates an exemplary graph of curves representative of percentages of feature space that are maximized or minimized along some dimension as the number of features used in the classifier expands, according to one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### I. Overview

### I.A. Overview of Methylation

In accordance with the present description, cfDNA fragments from an individual are treated, for example by converting unmethylated cytosines to uracils, sequenced and the sequence reads compared to a reference genome to identify the methylation states at specific CpG sites within the DNA fragments. Each CpG site may be methylated or unmethylated. Identification of anomalously methylated fragments, in comparison to healthy individuals, may provide insight into a subject's cancer status. As is well known in the art, DNA methylation anomalies (compared to healthy controls) can cause different effects, which may contribute to cancer. Various challenges arise in the identification of anomalously methylated cfDNA fragments. First off, determining a DNA fragment to be anomalously methylated can hold weight in comparison with a group of control individuals, such that if the control group is small in number, the determination loses confidence due to statistical variability within the smaller size of the control group. Additionally, among a group of control individuals, methylation status can vary which can be difficult to account for when determining a subject's DNA fragments to be anomalously methylated. On another note, methylation of a cytosine at a CpG site can causally influence methylation at a subsequent CpG site. To encapsulate this dependency can be another challenge in itself.

Methylation can typically occur in deoxyribonucleic acid (DNA) when a hydrogen atom on the pyrimidine ring of a cytosine base is converted to a methyl group, forming 5-methylcytosine. In particular, methylation can occur at dinucleotides of cytosine and guanine referred to herein as “CpG sites”. In other instances, methylation may occur at a cytosine not part of a CpG site or at another nucleotide that is not cytosine; however, these are rarer occurrences. In this present disclosure, methylation is discussed in reference to CpG sites for the sake of clarity. Anomalous DNA methylation can be identified as hypermethylation or hypomethylation, both of which may be indicative of cancer status. Throughout this disclosure, hypermethylation and hypomethylation can be characterized for a DNA fragment, if the DNA fragment comprises more than a threshold number of CpG sites with more than a threshold percentage of those CpG sites being methylated or unmethylated.

The principles described herein can be equally applicable for the detection of methylation in a non-CpG context, including non-cytosine methylation. In such embodiments, the wet laboratory assay used to detect methylation may vary from those described herein. Further, the methylation state vectors discussed herein may contain elements that are generally sites where methylation has or has not occurred (even if those sites are not CpG sites specifically). With that substitution, the remainder of the processes described herein can be the same, and consequently the inventive concepts described herein can be applicable to those other forms of methylation.

### I.B. Definitions

The term “cell free nucleic acid” or “cfNA” refers to nucleic acid fragments that circulate in an individual's body (e.g., blood) and originate from one or more healthy cells and/or from one or more unhealthy cells (e.g., cancer cells). The term “cell free DNA,” or “cfDNA” refers to deoxyribonucleic acid fragments that circulate in an individual's body (e.g., blood). Additionally, cfNAs or cfDNA in an individual's body may come from other non-human sources.

The term “genomic nucleic acid,” “genomic DNA,” or “gDNA” refers to nucleic acid molecules or deoxyribonucleic acid molecules obtained from one or more cells. In various embodiments, gDNA can be extracted from healthy cells (e.g., non-tumor cells) or from tumor cells (e.g., a biopsy sample). In some embodiments, gDNA can be extracted from a cell derived from a blood cell lineage, such as a white blood cell.

The term “circulating tumor DNA” or “ctDNA” refers to nucleic acid fragments that originate from tumor cells or other types of cancer cells, and which may be released into a bodily fluid of an individual (e.g., blood, sweat, urine, or saliva) as result of biological processes such as apoptosis or necrosis of dying cells or actively released by viable tumor cells.

The term “DNA fragment,” “fragment,” or “DNA molecule” may generally refer to any deoxyribonucleic acid fragments, i.e., cfDNA, gDNA, ctDNA, etc.

The term “anomalous fragment,” “anomalously methylated fragment,” or “fragment with an anomalous methylation pattern” refers to a fragment that has anomalous methylation of CpG sites. Anomalous methylation of a fragment may be determined using probabilistic models to identify unexpectedness of observing a fragment's methylation pattern in a control group.

The term “unusual fragment with extreme methylation” or “UFXM” refers to a hypomethylated fragment or a hypermethylated fragment. A hypomethylated fragment and a hypermethylated fragment refers to a fragment with at least some number of CpG sites (e.g., 5) that have over some threshold percentage (e.g., 90%) of methylation or unmethylation, respectively.

The term “anomaly score” refers to a score for a CpG site based on a number of anomalous fragments (or, in some embodiments, UFXMs) from a sample overlaps that CpG site. The anomaly score is used in context of featurization of a sample for classification.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

As used herein, the term “biological sample,” “patient sample,” or “sample” refers to any sample taken from a subject, which can reflect a biological state associated with the subject, and that includes cell-free DNA. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “control,” “control sample,” “reference,” “reference sample,” “normal,” and “normal sample” describe a sample from a subject that does not have a particular condition, or is otherwise healthy. In an example, a method as disclosed herein can be performed on a subject having a tumor, where the reference sample is a sample taken from a healthy tissue of the subject. A reference sample can be obtained from the subject, or from a database. The reference can be, e.g., a reference genome that is used to map nucleic acid fragment sequences obtained from sequencing a sample from the subject. A reference genome can refer to a haploid or diploid genome to which nucleic acid fragment sequences from the biological sample and a constitutional sample can be aligned and compared. An example of a constitutional sample can be DNA of white blood cells obtained from the subject. For a haploid genome, there can be only one nucleotide at each locus. For a diploid genome, heterozygous loci can be identified; each heterozygous locus can have two alleles, where either allele can allow a match for alignment to the locus.

As used herein, the term “cancer” or “tumor” refers to an abnormal mass of tissue in which the growth of the mass surpasses and is not coordinated with the growth of normal tissue.

As used herein, the phrase “healthy,” refers to a subject possessing good health. A healthy subject can demonstrate an absence of any malignant or non-malignant disease. A “healthy individual” can have other diseases or conditions, unrelated to the condition being assayed, which can normally not be considered “healthy.”

As used herein, the term “methylation” refers to a modification of deoxyribonucleic acid (DNA) where a hydrogen atom on the pyrimidine ring of a cytosine base is converted to a methyl group, forming 5-methylcytosine. In particular, methylation tends to occur at dinucleotides of cytosine and guanine referred to herein as “CpG sites.” In other instances, methylation may occur at a cytosine not part of a CpG site or at another nucleotide that's not cytosine; however, these are rarer occurrences. Anomalous cfDNA methylation can be identified as hypermethylation or hypomethylation, both of which may be indicative of cancer status. DNA methylation anomalies (compared to healthy controls) can cause different effects, which may contribute to cancer. The principles described herein are equally applicable for the detection of methylation in a CpG context and non-CpG context, including non-cytosine methylation. Further, the methylation state vectors may contain elements that are generally vectors of sites where methylation has or has not occurred (even if those sites are not CpG sites specifically).

As used interchangeably herein, the term “methylation fragment” or “nucleic acid methylation fragment” refers to a sequence of methylation states for each CpG site in a plurality of CpG sites, determined by a methylation sequencing of nucleic acids (e.g., a nucleic acid molecule and/or a nucleic acid fragment). In a methylation fragment, a location and methylation state for each CpG site in the nucleic acid fragment is determined based on the alignment of the sequence reads (e.g., obtained from sequencing of the nucleic acids) to a reference genome. A nucleic acid methylation fragment comprises a methylation state of each CpG site in a plurality of CpG sites (e.g., a methylation state vector), which specifies the location of the nucleic acid fragment in a reference genome (e.g., as specified by the position of the first CpG site in the nucleic acid fragment using a CpG index, or another similar metric) and the number of CpG sites in the nucleic acid fragment. Alignment of a sequence read to a reference genome, based on a methylation sequencing of a nucleic acid molecule, can be performed using a CpG index. As used herein, the term “CpG index” refers to a list of each CpG site in the plurality of CpG sites (e.g., CpG 1, CpG 2, CpG 3, etc.) in a reference genome, such as a human reference genome, which can be in electronic format. The CpG index further comprises a corresponding genomic location, in the corresponding reference genome, for each respective CpG site in the CpG index. Each CpG site in each respective nucleic acid methylation fragment is thus indexed to a specific location in the respective reference genome, which can be determined using the CpG index.

As used herein, the term “true positive” (TP) refers to a subject having a condition. “True positive” can refer to a subject that has a tumor, a cancer, a pre-cancerous condition (e.g., a pre-cancerous lesion), a localized or a metastasized cancer, or a non-malignant disease. “True positive” can refer to a subject having a condition, and is identified as having the condition by an assay or method of the present disclosure. As used herein, the term “true negative” (TN) refers to a subject that does not have a condition or does not have a detectable condition. True negative can refer to a subject that does not have a disease or a detectable disease, such as a tumor, a cancer, a pre-cancerous condition (e.g., a pre-cancerous lesion), a localized or a metastasized cancer, a non-malignant disease, or a subject that is otherwise healthy. True negative can refer to a subject that does not have a condition or does not have a detectable condition, or is identified as not having the condition by an assay or method of the present disclosure.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. Exemplary human reference genomes include but are not limited to NCBI build 34 (UCSC equivalent: hg16), NCBI build 35 (UCSC equivalent: hg17), NCBI build 36.1 (UCSC equivalent: hg18), GRCh37 (UCSC equivalent: hg19), and GRCh38 (UCSC equivalent: hg38).

As used herein, the term “sequence reads” or “reads” refers to nucleotide sequences produced by any sequencing process described herein or known in the art. Reads can be generated from one end of nucleic acid fragments (“single-end reads”), and sometimes are generated from both ends of nucleic acids (e.g., paired-end reads, double-end reads). In some embodiments, sequence reads (e.g., single-end or paired-end reads) can be generated from one or both strands of a targeted nucleic acid fragment. The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the terms “sequencing” and the like as used herein refers generally to any and all biochemical processes that may be used to determine the order of biological macromolecules such as nucleic acids or proteins. For example, sequencing data can include all or a portion of the nucleotide bases in a nucleic acid molecule such as a DNA fragment.

As used herein, the term “sequencing depth,” is interchangeably used with the term “coverage” and refers to the number of times a locus is covered by a consensus sequence read corresponding to a unique nucleic acid target molecule aligned to the locus; e.g., the sequencing depth is equal to the number of unique nucleic acid target molecules covering the locus. The locus can be as small as a nucleotide, or as large as a chromosome arm, or as large as an entire genome. Sequencing depth can be expressed as “Yx”, e.g., 50×, 100×, etc., where “Y” refers to the number of times a locus is covered with a sequence corresponding to a nucleic acid target; e.g., the number of times independent sequence information is obtained covering the particular locus. In some embodiments, the sequencing depth corresponds to the number of genomes that have been sequenced. Sequencing depth can also be applied to multiple loci, or the whole genome, in which case Y can refer to the mean or average number of times a locus or a haploid genome, or a whole genome, respectively, is sequenced. When a mean depth is quoted, the actual depth for different loci included in the dataset can span over a range of values. Ultra-deep sequencing can refer to at least 100× in sequencing depth at a locus.

As used herein, the term “sensitivity” or “true positive rate” (TPR) refers to the number of true positives divided by the sum of the number of true positives and false negatives. Sensitivity can characterize the ability of an assay or method to correctly identify a proportion of the population that truly has a condition. For example, sensitivity can characterize the ability of a method to correctly identify the number of subjects within a population having cancer. In another example, sensitivity can characterize the ability of a method to correctly identify the one or more markers indicative of cancer.

As used herein, the term “specificity” or “true negative rate” (TNR) refers to the number of true negatives divided by the sum of the number of true negatives and false positives. Specificity can characterize the ability of an assay or method to correctly identify a proportion of the population that truly does not have a condition. For example, specificity can characterize the ability of a method to correctly identify the number of subjects within a population not having cancer. In another example, specificity characterizes the ability of a method to correctly identify one or more markers indicative of cancer.

As used herein, the term “subject” refers to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. In some embodiments, a subject is a male or female of any stage (e.g., a man, a woman or a child). A subject from whom a sample is taken, or is treated by any of the methods or compositions described herein can be of any age and can be an adult, infant or child.

As used herein, the term “tissue” can correspond to a group of cells that group together as a functional unit. More than one type of cell can be found in a single tissue. Different types of tissue may consist of different types of cells (e.g., hepatocytes, alveolar cells or blood cells), but also can correspond to tissue from different organisms (mother vs. fetus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” can be used to refer to a tissue from which a cell-free nucleic acid originates. In one example, viral nucleic acid fragments can be derived from blood tissue. In another example, viral nucleic acid fragments can be derived from tumor tissue.

As used herein, the term “genomic” refers to a characteristic of the genome of an organism. Examples of genomic characteristics include, but are not limited to, those relating to the primary nucleic acid sequence of all or a portion of the genome (e.g., the presence or absence of a nucleotide polymorphism, indel, sequence rearrangement, mutational frequency, etc.), the copy number of one or more particular nucleotide sequences within the genome (e.g., copy number, allele frequency fractions, single chromosome or entire genome ploidy, etc.), the epigenetic status of all or a portion of the genome (e.g., covalent nucleic acid modifications such as methylation, histone modifications, nucleosome positioning, etc.), the expression profile of the organism's genome (e.g., gene expression levels, isotype expression levels, gene expression ratios, etc.).

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

### II. Sample Processing

II.A. Generating Methylation State Vectors for DNA Fragments

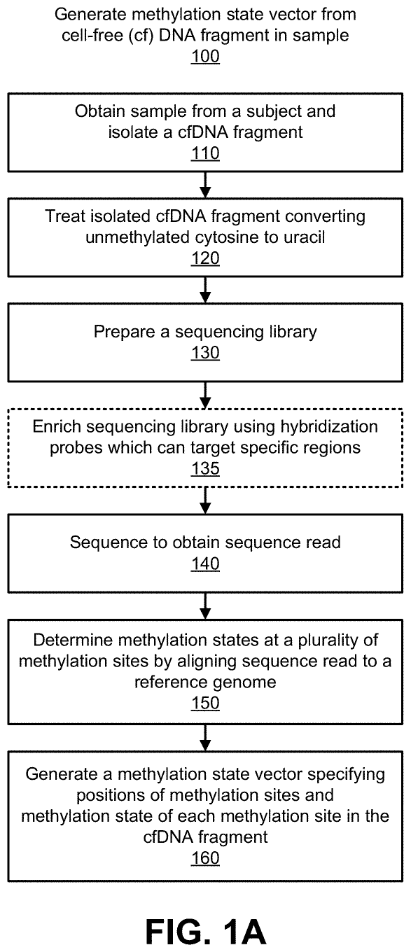

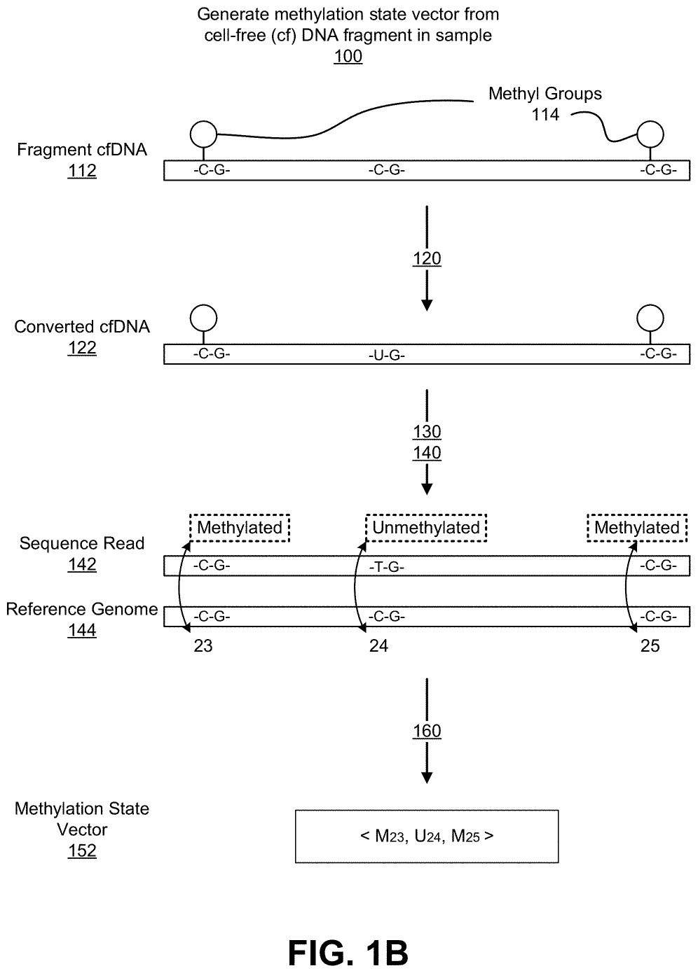

FIG. 1A is an exemplary flowchart describing a process 100 of sequencing a fragment of cell-free (cf) DNA to obtain a methylation state vector, according to one or more embodiments. In order to analyze DNA methylation, an analytics system first obtains 110 a sample from an individual comprising a plurality of cfDNA molecules. Generally, samples may be from healthy individuals, subjects known to have or suspected of having cancer, or subjects where no prior information is known. The test sample may be a sample selected from the group consisting of blood, plasma, serum, urine, fecal, and saliva samples. Alternatively, the test sample may comprise a sample selected from the group consisting of whole blood, a blood fraction (e.g., white blood cells (WBCs)), a tissue biopsy, pleural fluid, pericardial fluid, cerebral spinal fluid, and peritoneal fluid. In additional embodiments, the process 100 may be applied to sequence other types of DNA molecules.

From the sample, the analytics system can isolate each cfDNA molecule. The cfDNA molecules can be treated to convert unmethylated cytosines to uracils. In one embodiment, the method uses a bisulfite treatment of the DNA which converts the unmethylated cytosines to uracils without converting the methylated cytosines. For example, a commercial kit such as the EZ DNA Methylation™—Gold, EZ DNA Methylation™—Direct or an EZ DNA Methylation™—Lightning kit (available from Zymo Research Corp (Irvine, Calif.)) is used for the bisulfite conversion. In another embodiment, the conversion of unmethylated cytosines to uracils is accomplished using an enzymatic reaction. For example, the conversion can use a commercially available kit for conversion of unmethylated cytosines to uracils, such as APOBEC-Seq (NEBiolabs, Ipswich, Mass.).

From the converted cfDNA molecules, a sequencing library can be prepared 130. During library preparation, unique molecular identifiers (UMI) can be added to the nucleic acid molecules (e.g., DNA molecules) through adapter ligation. The UMIs can be short nucleic acid sequences (e.g., 4-10 base pairs) that are added to ends of DNA fragments (e.g., DNA molecules fragmented by physical shearing, enzymatic digestion, and/or chemical fragmentation) during adapter ligation. UMIs can be degenerate base pairs that serve as a unique tag that can be used to identify sequence reads originating from a specific DNA fragment. During PCR amplification following adapter ligation, the UMIs can be replicated along with the attached DNA fragment. This can provide a way to identify sequence reads that came from the same original fragment in downstream analysis.

Optionally, the sequencing library may be enriched 135 for cfDNA molecules, or genomic regions, that are informative for cancer status using a plurality of hybridization probes. The hybridization probes are short oligonucleotides capable of hybridizing to particularly specified cfDNA molecules, or targeted regions, and enriching for those fragments or regions for subsequent sequencing and analysis. Hybridization probes may be used to perform a targeted, high-depth analysis of a set of specified CpG sites of interest to the researcher. Hybridization probes can be tiled across one or more target sequences at a coverage of 1×, 2×, 3×, 4×, 5×, 6×, 7×, 8×, 9×, 10×, or more than 10×. For example, hybridization probes tiled at a coverage of 2× comprises overlapping probes such that each portion of the target sequence is hybridized to 2 independent probes. Hybridization probes can be tiled across one or more target sequences at a coverage of less than 1×.

In one embodiment, the hybridization probes are designed to enrich for DNA molecules that have been treated (e.g., using bisulfite) for conversion of unmethylated cytosines to uracils. During enrichment, hybridization probes (also referred to herein as “probes”) can be used to target and pull down nucleic acid fragments informative for the presence or absence of cancer (or disease), cancer status, or a cancer classification (e.g., cancer class or tissue of origin). The probes may be designed to anneal (or hybridize) to a target (complementary) strand of DNA. The target strand may be the “positive” strand (e.g., the strand transcribed into mRNA, and subsequently translated into a protein) or the complementary “negative” strand. The probes may range in length from 10s, 100s, or 1000s of base pairs. The probes can be designed based on a methylation site panel. The probes can be designed based on a panel of targeted genes to analyze particular mutations or target regions of the genome (e.g., of the human or another organism) that are suspected to correspond to certain cancers or other types of diseases. Moreover, the probes may cover overlapping portions of a target region.

Once prepared, the sequencing library or a portion thereof can be sequenced to obtain a plurality of sequence reads. The sequence reads may be in a computer-readable, digital format for processing and interpretation by computer software. The sequence reads may be aligned to a reference genome to determine alignment position information. The alignment position information may indicate a beginning position and an end position of a region in the reference genome that corresponds to a beginning nucleotide base and end nucleotide base of a given sequence read. Alignment position information may also include sequence read length, which can be determined from the beginning position and end position. A region in the reference genome may be associated with a gene or a segment of a gene. A sequence read can be comprised of a read pair denoted as R₁ and R₂. For example, the first read R₁ may be sequenced from a first end of a nucleic acid fragment whereas the second read R₂ may be sequenced from the second end of the nucleic acid fragment. Therefore, nucleotide base pairs of the first read R₁ and second read R₂ may be aligned consistently (e.g., in opposite orientations) with nucleotide bases of the reference genome. Alignment position information derived from the read pair R₁ and R₂ may include a beginning position in the reference genome that corresponds to an end of a first read (e.g., R₁) and an end position in the reference genome that corresponds to an end of a second read (e.g., R₂). In other words, the beginning position and end position in the reference genome can represent the likely location within the reference genome to which the nucleic acid fragment corresponds. An output file having SAM (sequence alignment map) format or BAM (binary) format may be generated and output for further analysis such as methylation state determination.

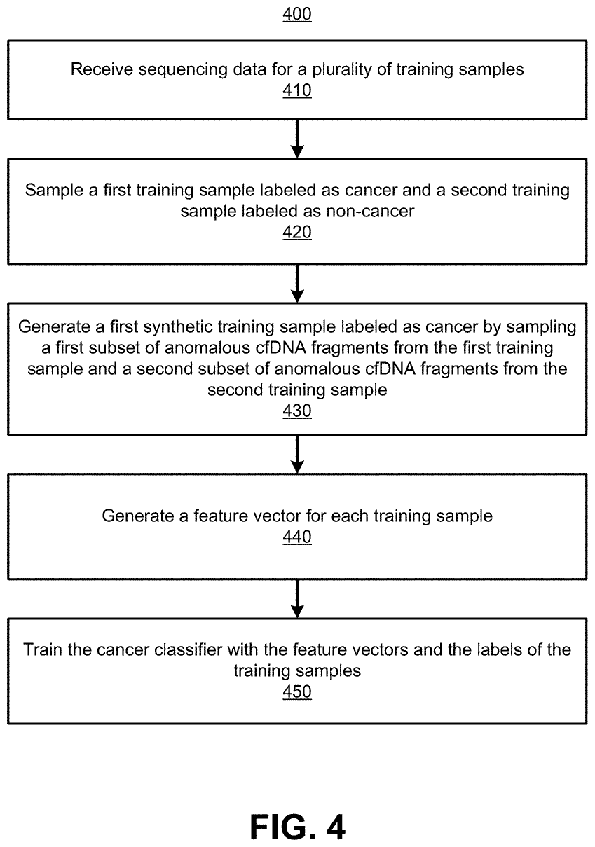

From the sequence reads, the analytics system determines 150 a location and methylation state for each CpG site based on alignment to a reference genome. The analytics system generates 160 a methylation state vector for each fragment specifying a location of the fragment in the reference genome (e.g., as specified by the position of the first CpG site in each fragment, or another similar metric), a number of CpG sites in the fragment, and the methylation state of each CpG site in the fragment whether methylated (e.g., denoted as M), unmethylated (e.g., denoted as U), or indeterminate (e.g., denoted as I). Observed states can be states of methylated and unmethylated; whereas, an unobserved state is indeterminate. Indeterminate methylation states may originate from sequencing errors and/or disagreements between methylation states of a DNA fragment's complementary strands. The methylation state vectors may be stored in temporary or persistent computer memory for later use and processing. Further, the analytics system may remove duplicate reads or duplicate methylation state vectors from a single sample. The analytics system may determine that a certain fragment with one or more CpG sites has an indeterminate methylation status over a threshold number or percentage, and may exclude such fragments or selectively include such fragments but build a model accounting for such indeterminate methylation statuses; one such model will be described below in conjunction with FIG. 4.

FIG. 1B is an exemplary illustration of the process 100 of FIG. 1A of sequencing a cfDNA molecule to obtain a methylation state vector, according to one or more embodiments. As an example, the analytics system receives a cfDNA molecule 112 that, in this example, contains three CpG sites. As shown, the first and third CpG sites of the cfDNA molecule 112 are methylated 114. During the treatment step 120, the cfDNA molecule 112 is converted to generate a converted cfDNA molecule 122. During the treatment 120, the second CpG site which was unmethylated has its cytosine converted to uracil. However, the first and third CpG sites were not converted.

After conversion, a sequencing library 130 is prepared and sequenced 140 to generate a sequence read 142. The analytics system aligns 150 the sequence read 142 to a reference genome 144. The reference genome 144 provides the context as to what position in a human genome the fragment cfDNA originates from. In this simplified example, the analytics system aligns 150 the sequence read 142 such that the three CpG sites correlate to CpG sites 23, 24, and 25 (arbitrary reference identifiers used for convenience of description). The analytics system can thus generate information both on methylation status of all CpG sites on the cfDNA molecule 112 and the position in the human genome that the CpG sites map to. As shown, the CpG sites on sequence read 142 which are methylated are read as cytosines. In this example, the cytosines appear in the sequence read 142 only in the first and third CpG site which allows one to infer that the first and third CpG sites in the original cfDNA molecule are methylated. Whereas, the second CpG site can be read as a thymine (U is converted to T during the sequencing process), and thus, one can infer that the second CpG site is unmethylated in the original cfDNA molecule. With these two pieces of information, the methylation status and location, the analytics system generates 160 a methylation state vector 152 for the fragment cfDNA 112. In this example, the resulting methylation state vector 152 is <M₂₃, U₂₄, M₂₅>, wherein M corresponds to a methylated CpG site, U corresponds to an unmethylated CpG site, and the subscript number corresponds to a position of each CpG site in the reference genome.

One or more alternative sequencing methods can be used for obtaining sequence reads from nucleic acids in a biological sample. The one or more sequencing methods can comprise any form of sequencing that can be used to obtain a number of sequence reads measured from nucleic acids (e.g., cell-free nucleic acids), including, but not limited to, high-throughput sequencing systems such as the Roche 454 platform, the Applied Biosystems SOLID platform, the Helicos True Single Molecule DNA sequencing technology, the sequencing-by-hybridization platform from Affymetrix Inc., the single-molecule, real-time (SMRT) technology of Pacific Biosciences, the sequencing-by-synthesis platforms from 454 Life Sciences, Illumina/Solexa and Helicos Biosciences, and the sequencing-by-ligation platform from Applied Biosystems. The ION TORRENT technology from Life technologies and Nanopore sequencing can also be used to obtain sequence reads from the nucleic acids (e.g., cell-free nucleic acids) in the biological sample. Sequencing-by-synthesis and reversible terminator-based sequencing (e.g., Illumina's Genome Analyzer; Genome Analyzer II; HISEQ 2000; HISEQ 2500 (Illumina, San Diego Calif.)) can be used to obtain sequence reads from the cell-free nucleic acid obtained from a biological sample of a training subject in order to form the genotypic dataset. Millions of cell-free nucleic acid (e.g., DNA) fragments can be sequenced in parallel. In one example of this type of sequencing technology, a flow cell is used that contains an optically transparent slide with eight individual lanes on the surfaces of which are bound oligonucleotide anchors (e.g., adaptor primers). A cell-free nucleic acid sample can include a signal or tag that facilitates detection. The acquisition of sequence reads from the cell-free nucleic acid obtained from the biological sample can include obtaining quantification information of the signal or tag via a variety of techniques such as, for example, flow cytometry, quantitative polymerase chain reaction (qPCR), gel electrophoresis, gene-chip analysis, microarray, mass spectrometry, cytofluorimetric analysis, fluorescence microscopy, confocal laser scanning microscopy, laser scanning cytometry, affinity chromatography, manual batch mode separation, electric field suspension, sequencing, and combination thereof.

The one or more sequencing methods can comprise a whole-genome sequencing assay. A whole-genome sequencing assay can comprise a physical assay that generates sequence reads for a whole genome or a substantial portion of the whole genome which can be used to determine large variations such as copy number variations or copy number aberrations. Such a physical assay may employ whole-genome sequencing techniques or whole-exome sequencing techniques. A whole-genome sequencing assay can have an average sequencing depth of at least 1×, 2×, 3×, 4×, 5×, 6×, 7×, 8×, 9×, 10×, at least 20×, at least 30×, or at least 40× across the genome of the test subject. In some embodiments, the sequencing depth is about 30,000×. The one or more sequencing methods can comprise a targeted panel sequencing assay. A targeted panel sequencing assay can have an average sequencing depth of at least 50,000×, at least 55,000×, at least 60,000×, or at least 70,000× sequencing depth for the targeted panel of genes. The targeted panel of genes can comprise between 450 and 500 genes. The targeted panel of genes can comprise a range of 500±5 genes, a range of 500±10 genes, or a range of 500±25 genes.

The one or more sequencing methods can comprise paired-end sequencing. The one or more sequencing methods can generate a plurality of sequence reads. The plurality of sequence reads can have an average length ranging between 10 and 700, between 50 and 400, or between 100 and 300. The one or more sequencing methods can comprise a methylation sequencing assay. The methylation sequencing can be i) whole-genome methylation sequencing or ii) targeted DNA methylation sequencing using a plurality of nucleic acid probes. For example, the methylation sequencing is whole-genome bisulfite sequencing (e.g., WGBS). The methylation sequencing can be a targeted DNA methylation sequencing using a plurality of nucleic acid probes targeting the most informative regions of the methylome, a unique methylation database and prior prototype whole-genome and targeted sequencing assays.

The methylation sequencing can detect one or more 5-methylcytosine (5mC) and/or 5-hydroxymethylcytosine (5hmC) in respective nucleic acid methylation fragments. The methylation sequencing can comprise conversion of one or more unmethylated cytosines or one or more methylated cytosines, in respective nucleic acid methylation fragments, to a corresponding one or more uracils. The one or more uracils can be detected during the methylation sequencing as one or more corresponding thymines. The conversion of one or more unmethylated cytosines or one or more methylated cytosines can comprise a chemical conversion, an enzymatic conversion, or combinations thereof.

For example, bisulfite conversion involves converting cytosine to uracil while leaving methylated cytosines (e.g., 5-methylcytosine or 5-mC) intact. In some DNA, about 95% of cytosines may not methylated in the DNA, and the resulting DNA fragments may include many uracils which are represented by thymines. Enzymatic conversion processes may be used to treat the nucleic acids prior to sequencing, which can be performed in various ways. One example of a bisulfite-free conversion comprises a bisulfite-free and base-resolution sequencing method, TET-assisted pyridine borane sequencing (TAPS), for non-destructive and direct detection of 5-methylcytosine and 5-hydroxymethylcytosine without affecting unmodified cytosines. The methylation state of a CpG site in the corresponding plurality of CpG sites in the respective nucleic acid methylation fragment can be methylated when the CpG site is determined by the methylation sequencing to be methylated, and unmethylated when the CpG site is determined by the methylation sequencing to not be methylated.

A methylation sequencing assay (e.g., WGBS and/or targeted methylation sequencing) can have an average sequencing depth including but not limited to up to about 1,000×, 2,000×, 3,000×, 5,000×, 10,000×, 15,000×, 20,000×, or 30,000×. The methylation sequencing can have a sequencing depth that is greater than 30,000×, e.g., at least 40,000× or 50,000×. A whole-genome bisulfite sequencing method can have an average sequencing depth of between 20× and 50×, and a targeted methylation sequencing method has an average effective depth of between 100× and 1000×, where effective depth can be the equivalent whole-genome bisulfite sequencing coverage for obtaining the same number of sequence reads obtained by targeted methylation sequencing.

For further details regarding methylation sequencing (e.g., WGBS and/or targeted methylation sequencing), see, e.g., U.S. Patent Application No. 62/642,480, entitled “Methylation Fragment Anomaly Detection,” filed Mar. 13, 2018, and U.S. patent application Ser. No. 16/719,902, entitled “Systems and Methods for Estimating Cell Source Fractions Using Methylation Information,” filed Dec. 18, 2019, each of which is hereby incorporated by reference. Other methods for methylation sequencing, including those disclosed herein and/or any modifications, substitutions, or combinations thereof, can be used to obtain fragment methylation patterns. A methylation sequencing can be used to identify one or more methylation state vectors, as described, for example, in U.S. patent application Ser. No. 16/352,602, entitled “Anomalous Fragment Detection and Classification,” filed Mar. 13, 2019, or in accordance with any of the techniques disclosed in U.S. patent application Ser. No. 15/931,022, entitled “Model-Based Featurization and Classification,” filed May 13, 2020, each of which is hereby incorporated by reference.

The methylation sequencing of nucleic acids and the resulting one or more methylation state vectors can be used to obtain a plurality of nucleic acid methylation fragments. Each corresponding plurality of nucleic acid methylation fragments (e.g., for each respective genotypic dataset) can comprise more than 100 nucleic acid methylation fragments. An average number of nucleic acid methylation fragments across each corresponding plurality of nucleic acid methylation fragments can comprise 1000 or more nucleic acid methylation fragments, 5000 or more nucleic acid methylation fragments, 10,000 or more nucleic acid methylation fragments, 20,000 or more nucleic acid methylation fragments, or 30,000 or more nucleic acid methylation fragments. An average number of nucleic acid methylation fragments across each corresponding plurality of nucleic acid methylation fragments can be between 10,000 nucleic acid methylation fragments and 50,000 nucleic acid methylation fragments. The corresponding plurality of nucleic acid methylation fragments can comprise one thousand or more, ten thousand or more, 100 thousand or more, one million or more, ten million or more, 100 million or more, 500 million or more, one billion or more, two billion or more, three billion or more, four billion or more, five billion or more, six billion or more, seven billion or more, eight billion or more, nine billion or more, or 10 billion or more nucleic acid methylation fragments. An average length of a corresponding plurality of nucleic acid methylation fragments can be between 140 and 280 nucleotides.

Further details regarding methods for sequencing nucleic acids and methylation sequencing data are disclosed in U.S. Provisional Patent Application No. 62/985,258, titled “Systems and Methods for Cancer Condition Determination Using Autoencoders,” filed Mar. 4, 2020, which is hereby incorporated herein by reference in its entirety.

II.B. Identifying Anomalous Fragments

The analytics system can determine anomalous fragments for a sample using the sample's methylation state vectors. For each fragment in a sample, the analytics system can determine whether the fragment is an anomalous fragment using the methylation state vector corresponding to the fragment. In some embodiments, the analytics system calculates a p-value score for each methylation state vector describing a probability of observing that methylation state vector or other methylation state vectors even less probable in the healthy control group. The process for calculating a p-value score is further discussed below in Section II.B.i. P-Value Filtering. The analytics system may determine fragments with a methylation state vector having below a threshold p-value score as anomalous fragments. In some embodiments, the analytics system further labels fragments with at least some number of CpG sites that have over some threshold percentage of methylation or unmethylation as hypermethylated and hypomethylated fragments, respectively. A hypermethylated fragment or a hypomethylated fragment may also be referred to as an unusual fragment with extreme methylation (UFXM). In other embodiments, the analytics system may implement various other probabilistic models for determining anomalous fragments. Examples of other probabilistic models include a mixture model, a deep probabilistic model, etc. In some embodiments, the analytics system may use any combination of the processes described below for identifying anomalous fragments. With the identified anomalous fragments, the analytics system may filter the set of methylation state vectors for a sample for use in other processes, e.g., for use in training and deploying a cancer classifier.

II.B.I. P-Value Filtering

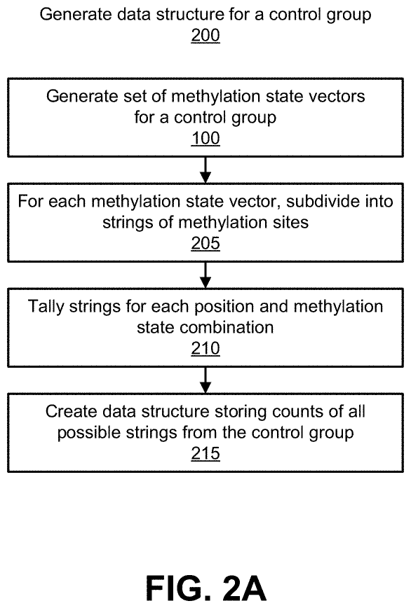

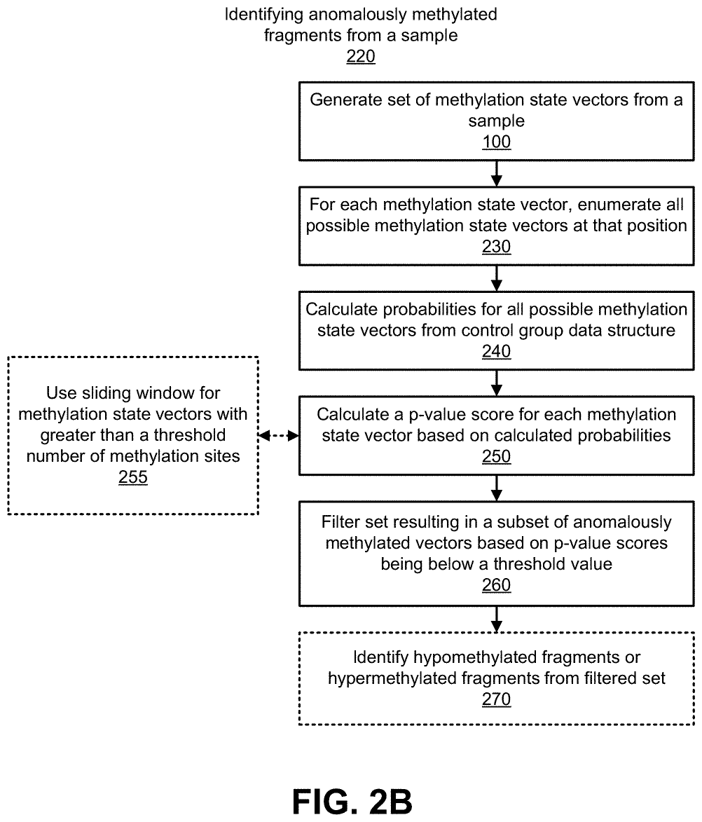

In some embodiments, the analytics system calculates a p-value score for each methylation state vector compared to methylation state vectors from fragments in a healthy control group. The p-value score can describe a probability of observing the methylation status matching that methylation state vector or other methylation state vectors even less probable in the healthy control group. In order to determine a DNA fragment to be anomalously methylated, the analytics system can use a healthy control group with a majority of fragments that are normally methylated. When conducting this probabilistic analysis for determining anomalous fragments, the determination can hold weight in comparison with the group of control subjects that make up the healthy control group. To ensure robustness in the healthy control group, the analytics system may select some threshold number of healthy individuals to source samples including DNA fragments. FIG. 2A below describes the method of generating a data structure for a healthy control group with which the analytics system may calculate p-value scores. FIG. 2B describes the method of calculating a p-value score with the generated data structure.

FIG. 2A is a flowchart describing a process 200 of generating a data structure for a healthy control group, according to an embodiment. To create a healthy control group data structure, the analytics system can receive a plurality of DNA fragments (e.g., cfDNA) from a plurality of healthy individuals. A methylation state vector can be identified for each fragment, for example via the process 100.

With each fragment's methylation state vector, the analytics system can subdivide 205 the methylation state vector into strings of CpG sites. In some embodiments, the analytics system subdivides 205 the methylation state vector such that the resulting strings are all less than a given length. For example, a methylation state vector of length 11 may be subdivided into strings of length less than or equal to 3 would result in 9 strings of length 3, 10 strings of length 2, and 11 strings of length 1. In another example, a methylation state vector of length 7 being subdivided into strings of length less than or equal to 4 can result in 4 strings of length 4, 5 strings of length 3, 6 strings of length 2, and 7 strings of length 1. If a methylation state vector is shorter than or the same length as the specified string length, then the methylation state vector may be converted into a single string containing all of the CpG sites of the vector.

The analytics system tallies 210 the strings by counting, for each possible CpG site and possibility of methylation states in the vector, the number of strings present in the control group having the specified CpG site as the first CpG site in the string and having that possibility of methylation states. For example, at a given CpG site and considering string lengths of 3, there are 2\hat{ }3 or 8 possible string configurations. At that given CpG site, for each of the 8 possible string configurations, the analytics system tallies 210 how many occurrences of each methylation state vector possibility come up in the control group. Continuing this example, this may involve tallying the following quantities: <Mₓ, Mₓ₊₁, Mₓ₊₂>, <Mₓ, Mₓ₊₁, Uₓ₊₂>, . . . , <Uₓ, Uₓ₊₁, Uₓ₊₂> for each starting CpG site x in the reference genome. The analytics system creates 215 the data structure storing the tallied counts for each starting CpG site and string possibility.

There are several benefits to setting an upper limit on string length. First, depending on the maximum length for a string, the size of the data structure created by the analytics system can dramatically increase in size. For instance, maximum string length of 4 means that every CpG site has at the very least 2\hat{ }4 numbers to tally for strings of length 4. Increasing the maximum string length to 5 means that every CpG site has an additional 2\hat{ }4 or 16 numbers to tally, doubling the numbers to tally (and computer memory required) compared to the prior string length. Reducing string size can help keep the data structure creation and performance (e.g., use for later accessing as described below), in terms of computational and storage, reasonable. Second, a statistical consideration to limiting the maximum string length can be to avoid overfitting downstream models that use the string counts. If long strings of CpG sites do not, biologically, have a strong effect on the outcome (e.g., predictions of anomalousness that predictive of the presence of cancer), calculating probabilities based on large strings of CpG sites can be problematic as it uses a significant amount of data that may not be available, and thus can be too sparse for a model to perform appropriately. For example, calculating a probability of anomalousness/cancer conditioned on the prior 100 CpG sites can use counts of strings in the data structure of length 100, ideally some matching exactly the prior 100 methylation states. If only sparse counts of strings of length 100 are available, there can be insufficient data to determine whether a given string of length of 100 in a test sample is anomalous or not.

FIG. 2B is a flowchart describing a process 220 for identifying anomalously methylated fragments from an individual, according to an embodiment. In process 220, the analytics system generates 100 methylation state vectors from cfDNA fragments of the subject. The analytics system can handle each methylation state vector as follows.

For a given methylation state vector, the analytics system enumerates 230 all possibilities of methylation state vectors having the same starting CpG site and same length (i.e., set of CpG sites) in the methylation state vector. As each methylation state is generally either methylated or unmethylated there can be effectively two possible states at each CpG site, and thus the count of distinct possibilities of methylation state vectors can depend on a power of 2, such that a methylation state vector of length n would be associated with 2ⁿ possibilities of methylation state vectors. With methylation state vectors inclusive of indeterminate states for one or more CpG sites, the analytics system may enumerate 230 possibilities of methylation state vectors considering only CpG sites that have observed states.

The analytics system calculates 240 the probability of observing each possibility of methylation state vector for the identified starting CpG site and methylation state vector length by accessing the healthy control group data structure. In some embodiments, calculating the probability of observing a given possibility uses a Markov chain probability to model the joint probability calculation. The Markov model can be trained, at least in part, based upon evaluation of a methylation state of each CpG site in the corresponding plurality of CpG sites of the respective fragment (e.g., nucleic acid methylation fragment) across those nucleic acid methylation fragments in a healthy noncancer cohort dataset that have the corresponding plurality of CpG sites. For example, a Markov model (e.g., a Hidden Markov Model or HMM) is used to determine the probability that a sequence of methylation states (comprising, e.g., “M” or “U”) can be observed for a nucleic acid methylation fragment in a plurality of nucleic acid methylation fragments, given a set of probabilities that determine, for each state in the sequence, the likelihood of observing the next state in the sequence. The set of probabilities can be obtained by training the HMM. Such training can involve computing statistical parameters (e.g., the probability that a first state can transition to a second state (the transition probability) and/or the probability that a given methylation state can be observed for a respective CpG site (the emission probability)), given an initial training dataset of observed methylation state sequences (e.g., methylation patterns). HMIs can be trained using supervised training (e.g., using samples where the underlying sequence as well as the observed states are known) and/or unsupervised training (e.g., Viterbi learning, maximum likelihood estimation, expectation-maximization training, and/or Baum-Welch training). In other embodiments, calculation methods other than Markov chain probabilities are used to determine the probability of observing each possibility of methylation state vector. For example, such calculation method can include a learned representation. The p-value threshold can be between 0.01 and 0.10, or between 0.03 and 0.06. The p-value threshold can be 0.05. The p-value threshold can be less than 0.01, less than 0.001, or less than 0.0001.

The analytics system calculates 250 a p-value score for the methylation state vector using the calculated probabilities for each possibility. In some embodiments, this includes identifying the calculated probability corresponding to the possibility that matches the methylation state vector in question. Specifically, this can be the possibility having the same set of CpG sites, or similarly the same starting CpG site and length as the methylation state vector. The analytics system can sum the calculated probabilities of any possibilities having probabilities less than or equal to the identified probability to generate the p-value score.

This p-value can represent the probability of observing the methylation state vector of the fragment or other methylation state vectors even less probable in the healthy control group. A low p-value score can, thereby, generally correspond to a methylation state vector which is rare in a healthy individual, and which causes the fragment to be labeled anomalously methylated, relative to the healthy control group. A high p-value score can generally relate to a methylation state vector is expected to be present, in a relative sense, in a healthy individual. If the healthy control group is a non-cancerous group, for example, a low p-value can indicate that the fragment is anomalous methylated relative to the non-cancer group, and therefore possibly indicative of the presence of cancer in the test subject.

As above, the analytics system can calculate p-value scores for each of a plurality of methylation state vectors, each representing a cfDNA fragment in the test sample. To identify which of the fragments are anomalously methylated, the analytics system may filter 260 the set of methylation state vectors based on their p-value scores. In some embodiments, filtering is performed by comparing the p-values scores against a threshold and keeping only those fragments below the threshold. This threshold p-value score can be on the order of 0.1, 0.01, 0.001, 0.0001, or similar.

According to example results from the process 400, the analytics system can yield a median (range) of 2,800 (1,500-12,000) fragments with anomalous methylation patterns for participants without cancer in training, and a median (range) of 3,000 (1,200-220,000) fragments with anomalous methylation patterns for participants with cancer in training. These filtered sets of fragments with anomalous methylation patterns may be used for the downstream analyses as described below in Section III.

In some embodiments, the analytics system uses 255 a sliding window to determine possibilities of methylation state vectors and calculate p-values. Rather than enumerating possibilities and calculating p-values for entire methylation state vectors, the analytics system can enumerate possibilities and calculates p-values for only a window of sequential CpG sites, where the window is shorter in length (of CpG sites) than at least some fragments (otherwise, the window would serve no purpose). The window length may be static, user determined, dynamic, or otherwise selected.

In calculating p-values for a methylation state vector larger than the window, the window can identify the sequential set of CpG sites from the vector within the window starting from the first CpG site in the vector. The analytic system can calculate a p-value score for the window including the first CpG site. The analytics system can then “slide” the window to the second CpG site in the vector, and calculates another p-value score for the second window. Thus, for a window size l and methylation vector length m, each methylation state vector can generate m−l+1 p-value scores. After completing the p-value calculations for each portion of the vector, the lowest p-value score from all sliding windows can be taken as the overall p-value score for the methylation state vector. In other embodiments, the analytics system aggregates the p-value scores for the methylation state vectors to generate an overall p-value score.

Using the sliding window can help to reduce the number of enumerated possibilities of methylation state vectors and their corresponding probability calculations that would otherwise need to be performed. To give a realistic example, it can be for fragments to have upwards of 54 CpG sites. Instead of computing probabilities for 2\hat{ }54 (˜1.8×10\hat{ }16) possibilities to generate a single p-score, the analytics system can instead use a window of size 5 (for example) which results in 50 p-value calculations for each of the 50 windows of the methylation state vector for that fragment. Each of the 50 calculations can enumerate 2\hat{ }5 (32) possibilities of methylation state vectors, which total results in 50×2\hat{ }5 (1.6×10\hat{ }3) probability calculations. This can result in a vast reduction of calculations to be performed, with no meaningful hit to the accurate identification of anomalous fragments.

In embodiments with indeterminate states, the analytics system may calculate a p-value score summing out CpG sites with indeterminates states in a fragment's methylation state vector. The analytics system can identify all possibilities that have consensus with the all methylation states of the methylation state vector excluding the indeterminate states. The analytics system may assign the probability to the methylation state vector as a sum of the probabilities of the identified possibilities. As an example, the analytics system can calculate a probability of a methylation state vector of <M₁, I₂, U₃> as a sum of the probabilities for the possibilities of methylation state vectors of <M₁, M₂, U₃> and <M₁, U₂, U₃> since methylation states for CpG sites 1 and 3 are observed and in consensus with the fragment's methylation states at CpG sites 1 and 3. This method of summing out CpG sites with indeterminate states can use calculations of probabilities of possibilities up to 2\hat{ }i, wherein i denotes the number of indeterminate states in the methylation state vector. In additional embodiments, a dynamic programming algorithm may be implemented to calculate the probability of a methylation state vector with one or more indeterminate states. Advantageously, the dynamic programming algorithm operates in linear computational time.

In some embodiments, the computational burden of calculating probabilities and/or p-value scores may be further reduced by caching at least some calculations. For example, the analytic system may cache in transitory or persistent memory calculations of probabilities for possibilities of methylation state vectors (or windows thereof). If other fragments have the same CpG sites, caching the possibility probabilities can allow for efficient calculation of p-score values without needing to re-calculate the underlying possibility probabilities. Equivalently, the analytics system may calculate p-value scores for each of the possibilities of methylation state vectors associated with a set of CpG sites from vector (or window thereof). The analytics system may cache the p-value scores for use in determining the p-value scores of other fragments including the same CpG sites. Generally, the p-value scores of possibilities of methylation state vectors having the same CpG sites may be used to determine the p-value score of a different one of the possibilities from the same set of CpG sites.

One or more nucleic acid methylation fragments can be filtered prior to training region models or cancer classifier. Filtering nucleic acid methylation fragments can comprise removing, from the corresponding plurality of nucleic acid methylation fragments, each respective nucleic acid methylation fragment that fails to satisfy one or more selection criteria (e.g., below or above one selection criteria). The one or more selection criteria can comprise a p-value threshold. The output p-value of the respective nucleic acid methylation fragment can be determined, at least in part, based upon a comparison of the corresponding methylation pattern of the respective nucleic acid methylation fragment to a corresponding distribution of methylation patterns of those nucleic acid methylation fragments in a healthy noncancer cohort dataset that have the corresponding plurality of CpG sites of the respective nucleic acid methylation fragment.

Filtering a plurality of nucleic acid methylation fragments can comprise removing each respective nucleic acid methylation fragment that fails to satisfy a p-value threshold. The filter can be applied to the methylation pattern of each respective nucleic acid methylation fragment using the methylation patterns observed across the first plurality of nucleic acid methylation fragments. Each respective methylation pattern of each respective nucleic acid methylation fragment (e.g., Fragment One, . . . , Fragment N) can comprise a corresponding one or more methylation sites (e.g., CpG sites) identified with a methylation site identifier and a corresponding methylation pattern, represented as a sequence of l's and 0's, where each “1” represents a methylated CpG site in the one or more CpG sites and each “0” represents an unmethylated CpG site in the one or more CpG sites. The methylation patterns observed across the first plurality of nucleic acid methylation fragments can be used to build a methylation state distribution for the CpG site states collectively represented by the first plurality of nucleic acid methylation fragments (e.g., CpG site A, CpG site B, . . . , CpG site ZZZ). Further details regarding processing of nucleic acid methylation fragments are disclosed in U.S. Provisional Patent Application No. 62/985,258, titled “Systems and Methods for Cancer Condition Determination Using Autoencoders,” filed Mar. 4, 2020, which is hereby incorporated herein by reference in its entirety.

The respective nucleic acid methylation fragment may fail to satisfy a selection criterion in the one or more selection criteria when the respective nucleic acid methylation fragment has an anomalous methylation score that is less than an anomalous methylation score threshold. In this situation, the anomalous methylation score can be determined by a mixture model. For example, a mixture model can detect an anomalous methylation pattern in a nucleic acid methylation fragment by determining the likelihood of a methylation state vector (e.g., a methylation pattern) for the respective nucleic acid methylation fragment based on the number of possible methylation state vectors of the same length and at the same corresponding genomic location. This can be executed by generating a plurality of possible methylation states for vectors of a specified length at each genomic location in a reference genome. Using the plurality of possible methylation states, the number of total possible methylation states and subsequently the probability of each predicted methylation state at the genomic location can be determined. The likelihood of a sample nucleic acid methylation fragment corresponding to a genomic location within the reference genome can then be determined by matching the sample nucleic acid methylation fragment to a predicted (e.g., possible) methylation state and retrieving the calculated probability of the predicted methylation state. An anomalous methylation score can then be calculated based on the probability of the sample nucleic acid methylation fragment.

The respective nucleic acid methylation fragment can fail to satisfy a selection criterion in the one or more selection criteria when the respective nucleic acid methylation fragment has less than a threshold number of residues. The threshold number of residues can be between 10 and 50, between 50 and 100, between 100 and 150, or more than 150. The threshold number of residues can be a fixed value between 20 and 90. The respective nucleic acid methylation fragment may fail to satisfy a selection criterion in the one or more selection criteria when the respective nucleic acid methylation fragment has less than a threshold number of CpG sites. The threshold number of CpG sites can be 4, 5, 6, 7, 8, 9, or 10. The respective nucleic acid methylation fragment can fail to satisfy a selection criterion in the one or more selection criteria when a genomic start position and a genomic end position of the respective nucleic acid methylation fragment indicates that the respective nucleic acid methylation fragment represents less than a threshold number of nucleotides in a human genome reference sequence.

The filtering can remove a nucleic acid methylation fragment in the corresponding plurality of nucleic acid methylation fragments that has the same corresponding methylation pattern and the same corresponding genomic start position and genomic end position as another nucleic acid methylation fragment in the corresponding plurality of nucleic acid methylation fragments. This filtering step can remove redundant fragments that are exact duplicates, including, in some instances, PCR duplicates. The filtering can remove a nucleic acid methylation fragment that has the same corresponding genomic start position and genomic end position and less than a threshold number of different methylation states as another nucleic acid methylation fragment in the corresponding plurality of nucleic acid methylation fragments. The threshold number of different methylation states used for retention of a nucleic acid methylation fragment can be 1, 2, 3, 4, 5, or more than 5. For example, a first nucleic acid methylation fragment having the same corresponding genomic start and end position as a second nucleic acid methylation fragment but having at least 1, at least 2, at least 3, at least 4, or at least 5 different methylation states at a respective CpG site (e.g., aligned to a reference genome) is retained. As another example, a first nucleic acid methylation fragment having the same methylation state vector (e.g., methylation pattern) but different corresponding genomic start and end positions as a second nucleic acid methylation fragment is also retained.

The filtering can remove assay artifacts in the plurality of nucleic acid methylation fragments. The removal of assay artifacts can comprise removing sequence reads obtained from sequenced hybridization probes and/or sequence reads obtained from sequences that failed to undergo conversion during bisulfite conversion. The filtering can remove contaminants (e.g., due to sequencing, nucleic acid isolation, and/or sample preparation).

The filtering can remove a subset of methylation fragments from the plurality of methylation fragments based on mutual information filtering of the respective methylation fragments against the cancer state across the plurality of training subjects. For example, mutual information can provide a measure of the mutual dependence between two conditions of interest sampled simultaneously. Mutual information can be determined by selecting an independent set of CpG sites (e.g., within all or a portion of a nucleic acid methylation fragment) from one or more datasets and comparing the probability of the methylation states for the set of CpG sites between two sample groups (e.g., subsets and/or groups of genotypic datasets, biological samples, and/or subjects). A mutual information score can denote the probability of the methylation pattern for a first condition versus a second condition at the respective region in the respective frame of the sliding window, thus indicating the discriminative power of the respective region. A mutual information score can be similarly calculated for each region in each frame of the sliding window as it progresses across the selected sets of CpG sites and/or the selected genomic regions. Further details regarding mutual information filtering are disclosed in U.S. patent application Ser. No. 17/119,606, titled “Cancer Classification using Patch Convolutional Neural Networks,” filed Dec. 11, 2020, which is hereby incorporated herein by reference in its entirety.

II.B.II. Hypermethylated Fragments and Hypomethylated Fragments

In some embodiments, the analytics system determines anomalous fragments as fragments with over a threshold number of CpG sites and either with over a threshold percentage of the CpG sites methylated or with over a threshold percentage of CpG sites unmethylated; the analytics system identifies such fragments as hypermethylated fragments or hypomethylated fragments. Example thresholds for length of fragments (or CpG sites) include more than 3, 4, 5, 6, 7, 8, 9, 10, etc. Example percentage thresholds of methylation or unmethylation include more than 80%, 85%, 90%, or 95%, or any other percentage within the range of 50%-100%.

II.C. Example Analytics System

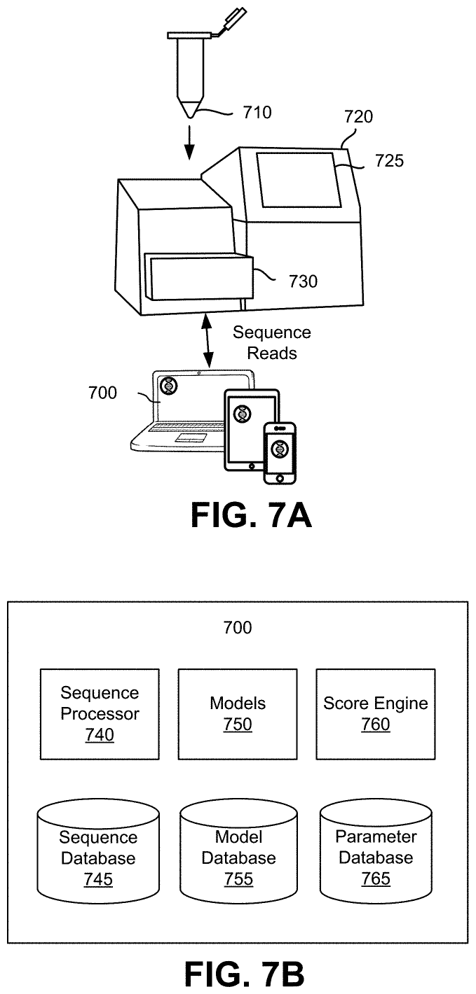

FIG. 7A is an exemplary flowchart of devices for sequencing nucleic acid samples according to one or more embodiments. This illustrative flowchart includes devices such as a sequencer 720 and an analytics system 700. The sequencer 720 and the analytics system 700 may work in tandem to perform one or more steps in the processes 100 of FIG. 1A, 200 of FIG. 2A, 220 of FIG. 2B, and other process described herein.

In various embodiments, the sequencer 720 receives an enriched nucleic acid sample 710. As shown in FIG. 7A, the sequencer 720 can include a graphical user interface 725 that enables user interactions with particular tasks (e.g., initiate sequencing or terminate sequencing) as well as one more loading stations 730 for loading a sequencing cartridge including the enriched fragment samples and/or for loading necessary buffers for performing the sequencing assays. Therefore, once a user of the sequencer 720 has provided the necessary reagents and sequencing cartridge to the loading station 730 of the sequencer 720, the user can initiate sequencing by interacting with the graphical user interface 725 of the sequencer 720. Once initiated, the sequencer 720 performs the sequencing and outputs the sequence reads of the enriched fragments from the nucleic acid sample 710.

In some embodiments, the sequencer 720 is communicatively coupled with the analytics system 700. The analytics system 700 includes some number of computing devices used for processing the sequence reads for various applications such as assessing methylation status at one or more CpG sites, variant calling or quality control. The sequencer 720 may provide the sequence reads in a BAM file format to the analytics system 700. The analytics system 700 can be communicatively coupled to the sequencer 720 through a wireless, wired, or a combination of wireless and wired communication technologies. Generally, the analytics system 700 is configured with a processor and non-transitory computer-readable storage medium storing computer instructions that, when executed by the processor, cause the processor to process the sequence reads or to perform one or more steps of any of the methods or processes disclosed herein.

In some embodiments, the sequence reads may be aligned to a reference genome using known methods in the art to determine alignment position information, e.g., via step 140 of the process 100 in FIG. 1A. Alignment position may generally describe a beginning position and an end position of a region in the reference genome that corresponds to a beginning nucleotide based and an end nucleotide base of a given sequence read. Corresponding to methylation sequencing, the alignment position information may be generalized to indicate a first CpG site and a last CpG site included in the sequence read according to the alignment to the reference genome. The alignment position information may further indicate methylation statuses and locations of all CpG sites in a given sequence read. A region in the reference genome may be associated with a gene or a segment of a gene; as such, the analytics system 700 may label a sequence read with one or more genes that align to the sequence read. In one embodiment, fragment length (or size) is be determined from the beginning and end positions.

In various embodiments, for example when a paired-end sequencing process is used, a sequence read is comprised of a read pair denoted as R_1 and R_2. For example, the first read R_1 may be sequenced from a first end of a double-stranded DNA (dsDNA) molecule whereas the second read R_2 may be sequenced from the second end of the double-stranded DNA (dsDNA). Therefore, nucleotide base pairs of the first read R_1 and second read R_2 may be aligned consistently (e.g., in opposite orientations) with nucleotide bases of the reference genome. Alignment position information derived from the read pair R_1 and R_2 may include a beginning position in the reference genome that corresponds to an end of a first read (e.g., R_1) and an end position in the reference genome that corresponds to an end of a second read (e.g., R_2). In other words, the beginning position and end position in the reference genome can represent the likely location within the reference genome to which the nucleic acid fragment corresponds. An output file having SAM (sequence alignment map) format or BAM (binary) format may be generated and output for further analysis.

Referring now to FIG. 7B, FIG. 7B is a block diagram of an analytics system 700 for processing DNA samples according to one embodiment. The analytics system implements one or more computing devices for use in analyzing DNA samples. The analytics system 700 includes a sequence processor 740, sequence database 745, model database 755, models 750, parameter database 765, and score engine 760. In some embodiments, the analytics system 700 performs some or all of the processes 100 of FIG. 1A and 200 of FIG. 2.

The sequence processor 740 generates methylation state vectors for fragments from a sample. At each CpG site on a fragment, the sequence processor 740 generates a methylation state vector for each fragment specifying a location of the fragment in the reference genome, a number of CpG sites in the fragment, and the methylation state of each CpG site in the fragment whether methylated, unmethylated, or indeterminate via the process 100 of FIG. 1A. The sequence processor 740 may store methylation state vectors for fragments in the sequence database 745. Data in the sequence database 745 may be organized such that the methylation state vectors from a sample are associated to one another.

Further, multiple different models 750 may be stored in the model database 755 or retrieved for use with test samples. In one example, a model is a trained cancer classifier for determining a cancer prediction for a test sample using a feature vector derived from anomalous fragments. The training and use of the cancer classifier will be further discussed in conjunction with Section III. Cancer Classifier for Determining Cancer. The analytics system 700 may train the one or more models 750 and store various trained parameters in the parameter database 765. The analytics system 700 stores the models 750 along with functions in the model database 755.

During inference, the score engine 760 uses the one or more models 750 to return outputs. The score engine 760 accesses the models 750 in the model database 755 along with trained parameters from the parameter database 765. According to each model, the score engine receives an appropriate input for the model and calculates an output based on the received input, the parameters, and a function of each model relating the input and the output. In some use cases, the score engine 760 further calculates metrics correlating to a confidence in the calculated outputs from the model. In other use cases, the score engine 760 calculates other intermediary values for use in the model.

### III. Cancer Classifier for Determining Cancer

III.A. Overview

The cancer classifier can be trained to receive a feature vector for a test sample and determine whether the test sample is from a test subject that has cancer or, more specifically, a particular cancer type. The cancer classifier can comprise a plurality of classification parameters and a function representing a relation between the feature vector as input and the cancer prediction as output determined by the function operating on the input feature vector with the classification parameters. In some embodiments, the feature vectors input into the cancer classifier are based on set of anomalous fragments determined from the test sample. The anomalous fragments may be determined via the process 220 in FIG. 2B, or more specifically hypermethylated and hypomethylated fragments as determined via the step 270 of the process 220, or anomalous fragments determined according to some other process. Prior to deployment of the cancer classifier, the analytics system can train the cancer classifier.

III.B. Generating Synthetic Training Samples

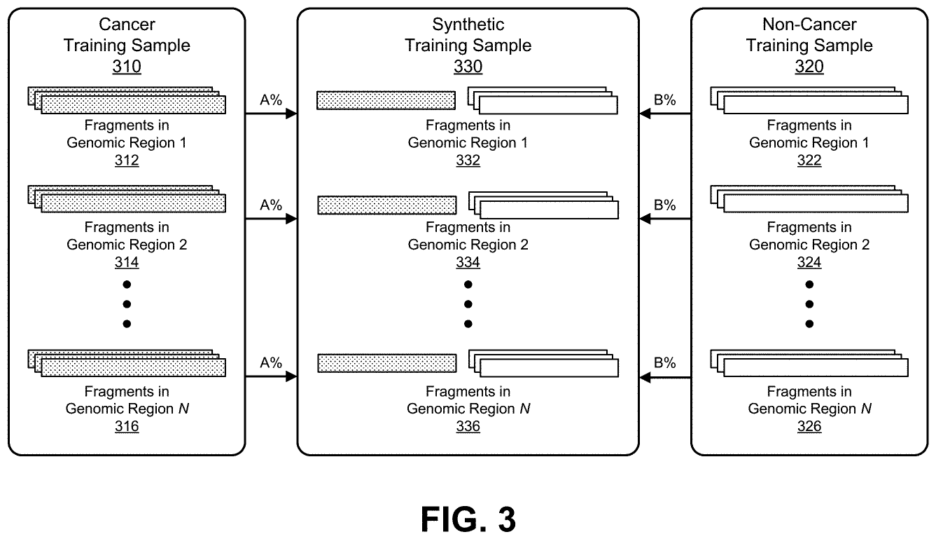

FIG. 3 illustrates an exemplary process of generating a synthetic training sample, according to one or more embodiments. The analytics system can use training samples obtained from individuals with known cancer statuses to generate one or more synthetic training samples. The analytics system can use the training samples including the synthetic training samples to train the cancer classifier.

The analytics system obtains a cancer training sample 310 and a non-cancer training sample 320 to generate a synthetic training sample 330. The cancer training sample 310 is derived from an individual with a known status of having cancer. The non-cancer training sample 320 is derived from an individual with a known status of not having cancer (“non-cancer”). Each training sample comprises cfDNA fragments that overlap at least one genomic region of a plurality of genomic regions in human genome. Given N number of genomic regions, cancer training sample 310 has fragments 312 in Genomic Region 1, fragments 314 in Genomic Region 2, and fragments for each Genomic Region up to fragments 316 in Genomic Region N. Similarly, non-cancer training sample 320 has fragments 322 in Genomic Region 1, fragments 324 in Genomic Region 2, and fragments for each Genomic Region up to fragments 326 in Genomic Region N.

The analytics system generates the synthetic training sample 330 by sampling fragments from the cancer training sample 310 and fragments from the non-cancer training sample 320. The analytics system, at each genomic region, samples a subset of fragments from the cancer training sample 310 at a first sampling probability and samples a subset of fragments from the non-cancer training sample 320 at a second sampling probability, complementary to the first sampling probability. As shown in the illustration, the first sampling probability is A % and the second sampling probability is B %. By sampling in this manner, the synthetic training sample 330 is generated to include A % of fragments 312 from the cancer training sample 310 and B % of fragments 322 from the non-cancer training sample 320 for Genomic Region 1. Similarly, the synthetic sample 330 is generated to include A % of fragments 314 from the cancer training sample 310 and B % of fragments 324 from the non-cancer training sample 320 for Genomic Region 2. This continues through the genomic regions up to Genomic Region N, where the synthetic sample 330 is generated to include A % of fragments 316 of cancer training sample 310 and B % of fragments 326 of non-cancer training sample 320 in Genomic Region N. The analytics system labels the synthetic training sample 330 with a label of cancer. The label may further include a particular cancer type present within the cancer training sample 310.

The sampling probabilities can be determined according to performance of the trained cancer classifier. The analytics system may train the cancer classifier and evaluate its performance. Performance of the classifier may include a limit of detection to predict presence of cancer in a sample at a minimal tumor fraction, i.e., the minimum percentage of cfDNA fragments shed from tumor tissue needed to detect cancer signal. For example, the classifier may have a limit of detection of one fragment shed from tumor tissue per thousand fragments in the sample. The first sampling probability corresponding to the percentage of fragments sampled from the cancer training sample 310 may be set to 0.001% (or around such a percentage). The analytics system may determine the second sampling probability as a complement to the first sampling probability. Complementary sampling probabilities have percentages that add up to 100%. For example, the complementary percentage of 0.001% is 0.999%, which is set as the second sampling probability corresponding to the percentage of fragments sampled from the non-cancer training sample 320. The analytics system may further adjust the sampling probabilities according to sequencing depth of the cancer training sample 310 and the non-cancer training sample 320. For example, the first sampling probability might be increased if the cancer training sample 310 has a smaller sequencing depth than the non-cancer training sample 320. The analytics system may progressively adjust the sampling probabilities as the cancer classifier is progressively trained with synthetic training samples.

FIG. 4 is an exemplary flowchart describing a process 400 of generating a synthetic training sample for training of a cancer classifier, according to one or more embodiments. Although the following description is in perspective of the analytics system, the following process may be performed by any of the components of the analytics system 700 shown in FIG. 7B.

The analytics system receives 410 sequencing data for a plurality of training samples. The analytics system can receive training samples each with a label of cancer or non-cancer. The training samples with a label of cancer may further have a label of a particular cancer type. Each training sample can comprise a plurality of cfDNA fragments that may be determined to be anomalously methylated according to the process 220 of FIG. 2B.

The analytics system samples 420 a first training sample labeled as cancer and a second training sample labeled as non-cancer. The first training sample may have an additional label of a particular cancer type of a plurality of cancer types.

The analytics system generates 430 a first synthetic training sample labeled as cancer by sampling a first subset of anomalous cfDNA fragments from the first training sample and a second subset of anomalous cfDNA fragments from the second training sample. As described in FIG. 3, the analytics system may sample fragments from each training sample according to genomic region by sampling probabilities. The analytics system, at each genomic region, can sample fragments in the genomic region for the first training sample according to a first sampling probability and samples fragments in the genomic region for the second training sample according to a second sampling probability, wherein the second sampling probability is complementary to the first sampling probability.

The analytics system may repeat steps 420 and 430 to generate additional synthetic training samples. A single cancer training sample may be used to generate multiple synthetic training samples labeled as cancer.

The analytic system generates 440 a feature vector for each training sample. The training samples include at least the first synthetic training sample and up to all synthetic training samples generated. The feature vector can be generated based on the anomalous cfDNA fragments in a training sample. One approach to featurization is described below in Section III.C. Training of Cancer Classifier.

The analytics system trains 450 the cancer classifier with the feature vectors and the labels of the training samples. The analytics system trains the cancer classifier by inputting the feature vectors of the training samples and adjusts parameters of the cancer classifier in optimization of the cancer classifier's predictive accuracy of the labels of the training samples. Further detail regarding training of the cancer classifier is described below in Section III.C. Training of Cancer Classifier.

Training the cancer classifier with the generated one or more synthetic training samples facilitates improved specificity and sensitivity of the cancer classifier. The improvement is attributable to several factors. For example, by using an expanded training set, data overfitting is reduced because the classifier can better generalize trends in the data. In addition, by determining the sampling probabilities, the generated synthetic training samples can have cancer signal near the limit of detection of the classifier. This, in turn, can allow for more robust training of the cancer classifier in feature space where cancer signal is scarcer.

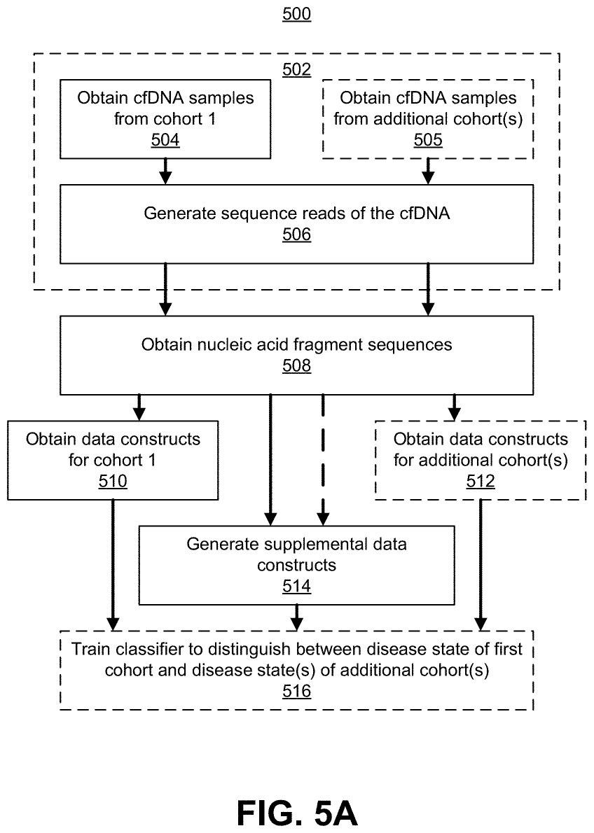

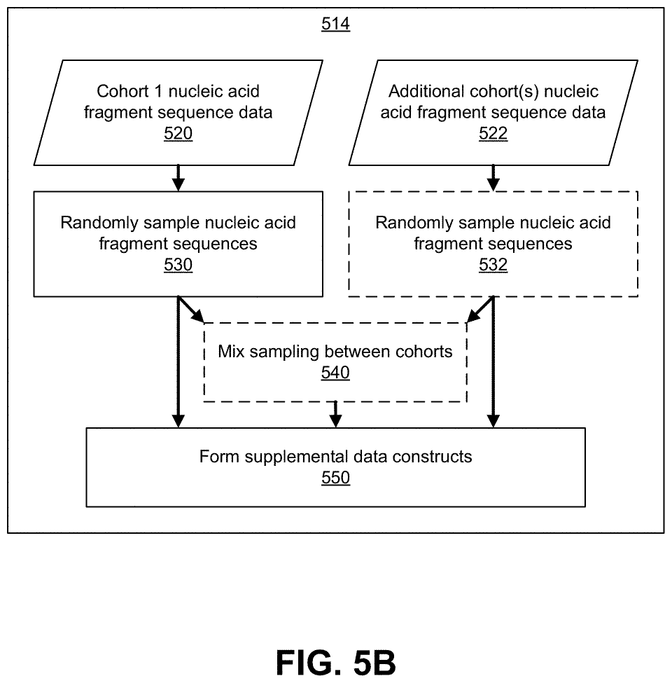

FIG. 5A illustrates an example workflow 500 for generating augmented data and optionally training a classifier to discriminate disease states from one another, in accordance with various embodiments of the present disclosure.

In some embodiments, the first step of workflow 500 is collection (502) of the underlying biological data from one or more training cohorts, e.g., where the subjects in each training cohort have a different disease state. Biological samples, e.g., containing nucleic acids, are collected (504) from subjects in a first cohort, each of whom has a first disease state, e.g., a particular state of cancer or cardiovascular disease for which cell-free nucleic acids are informative of the disease state. As illustrated in FIG. 2, biological samples are collected (505) from subjects in one or more additional cohorts, each of whom have a second disease state that is different from the first disease state. By way of example, subjects in the first cohort have cancer while subjects in a second cohort do not have cancer. Each biological sample used in the methods described herein can include cell-free nucleic acids, e.g., cfDNA. Advantageously, cell-free nucleic acids can be obtained by a minimally-invasive, small-volume blood draw from the subject, or possibly from non-invasive sampling of other bodily fluids such as saliva or urine. The systems and methods described herein can be suitable for evaluating any type of biological data that can be used to detect a disease state in a subject, e.g., cell-free or cellular genomic data, transcriptomic data, epigenetic data, proteomic data, metabolomic data, etc. The biological samples can be processed to obtain biological information about the subject (506). Cell-free nucleic acids (e.g., cfDNA) in the sample can be sequenced to generate cfDNA sequence reads.

Although workflow 500 illustrates optional steps of collecting a biological sample (e.g., obtaining cfDNA samples from cohort 1 (504) and other optional cohorts (505)) and biological feature extraction (e.g., generating cfDNA sequence reads 506), the methods described herein can begin by obtaining previously extracted biological features (e.g., sequence reads and optionally characteristics of the sequence reads) in electronic form.

Workflow 500 includes a step of obtaining (508) nucleic acid fragment sequences for nucleic acid samples from subjects in the first cohort and, optionally, from nucleic acid samples from subjects in additional cohorts. Workflow 500 further includes a step of obtaining (510) data constructs for each of the subjects in cohort 1, based on the biological information collected at step 506. The data constructs can include genomic features (or genomic characteristics), disease statuses, and optionally personal characteristics of the subjects. Examples of genomic features useful for the methods described herein include read counts (e.g., genomic copy number characteristics) which provide information about the relative abundance of particular sequences (e.g., genomic or exomic loci) in the biological sample, the presence of variant alleles (e.g., variant allele characteristics) which provide information about differences in the genome of the subject (e.g., in either or both of the germline or a diseased tissue) relative to a reference genome(s) for the species of the subject, allele frequencies (e.g., allelic ratio characteristics) which provide information about the relative abundance of variant alleles, relative to non-variant alleles, in the test biological sample, and methylation statuses (e.g., genomic methylation characteristics) which provide information about the methylation states of different genomic regions in the test biological sample. The particular features included in, and the formatting of, the data construct can be dictated by the classifier optionally trained in step 516 of workflow 500. In workflow 502, the nucleic acid fragment sequence data may not be merged together. In this situation, the identity of the source of the cfDNA can be maintained and each supplemental data construct can be constructed from the cfDNA of a single corresponding sample from one of the cohorts. In some alternative embodiments, the cfDNA from two or more samples of a cohort are merged into a single supplemental data construct.

Workflow 500 includes an optional step of obtaining (512) data constructs for each of the subjects in any additional cohorts, based on the biological information collected at step 506. The data constructs can include genotypic features, disease statuses, and optionally personal characteristics of the subjects, as described above. Where the data constructs are used to train a classifier to distinguish between the disease states of the subjects in the additional cohorts, the genomic features in the data constructs obtained at step 512 can be the same genomic features in the data constructs obtained for the first cohort at step 510.

Workflow 500 also includes a step of generating (514) supplemental data constructs containing augmented values for the genomic characteristics, based on a probabilistic sampling of the nucleic acid fragment sequences obtained for at least one subject in the first cohort. One or more of the supplemental data constructs can represent the state of a sample simulated to have a disease signature near the detection limit of the classifier being trained. In this situation, training of the classifier can be improved by presenting more examples of weak data signals representing a given disease state.

FIG. 5B illustrates an example workflow for generating supplemental data constructs at step 514. As illustrated in FIG. 5B, nucleic acid fragment sequence data 520 from one or more subjects in the first cohort (e.g., who have cancer) are probabilistically sampled (530) to select a subset of all nucleic acid fragment sequences that can then simulate data with a weaker disease signal. For instance, when starting from a normalized set of nucleic acid fragment sequences generated from a liquid biological sample having a tumor fraction of 0.2 (that is, 20% of the cell-free nucleic acids in the sample are from a cancerous cell), application of a 50% selection probability to each of the nucleic acid fragment sequences results in a selected set of nucleic acid fragment sequences for the corresponding supplemental data construct with approximately half the amount of cancer signal, which is roughly equivalent to the cancer signal expected of a sample having a tumor fraction of 0.1. In practice, a classifier can be trained using a cohort of cancer-free subjects and a cohort of cancer subjects, where the cancer subjects in the cohort vary with respect to tumor fraction. The performance of the trained classifier can then be evaluated in order to determine a limit of detection of the classifier. The trained classifier can be evaluated to determine the tumor fractions at which the performance of the classifier begins to substantially decline or fail altogether.

Then, method 502 (FIG. 2) can be used to generate supplemental data constructs centered on this tumor fraction. For instance, consider the case where the average cancer subject in the cancer cohort has a tumor fraction of 0.4 and that the trained classifier fails at tumor fraction 0.2. In this situation, the classifier may fail to identify subjects with a tumor fraction of 0.2 or below as having the cancer with adequate performance. In such instances, supplemental data constructs can be generated from the cancer cohort on a cohort subject-by-subject basis. For each subject, each of their fragment sequences can be selected for inclusion in a corresponding supplemental data construct on a probabilistic basis. Since the classifier is failed at 0.2, the supplemental data constructs with a tumor fraction can be in the vicinity of 0.2 in order to better train the classifier. So, for each respective subject in the cancer cohort, each of the nucleic acid fragment sequences can be selected for a corresponding supplemental data construct built using the nucleic acid fragment data in the cohort for the respective subject by probabilistically sampling (accepting) each nucleic acid fragment for inclusion in the corresponding supplemental data construct. In this example, a probabilistic sampling of 0.50 is applied to each nucleic acid fragment for the respective subject in the cancer cohort. Thus, if there are 1000 nucleic acid fragments for the respective subject in the cancer cohort, each nucleic acid fragment can be accepted into the corresponding supplemental data construct with fifty percent probability. Advantageously, without considering reference alleles and alternative alleles, or even knowing which alleles are determinative of the cancer signal, the raw count of discriminating alternative alleles can likely be halved by application of this probabilistic sampling in order to generate the corresponding supplemental data construct that simulates a real cohort sample having a tumor fraction of 0.2. The supplemental data constructs generated in this fashion can be combined with the original cohort data to once again train the classifier, now with more data, and the performance of the classifier against the original data can be once again evaluated. Advantageously, as shown in the examples below, this approach can improve classifier performance, particularly at lower tumor fraction where the original cohort data had a paucity of subjects.

Optionally, in alternative embodiments, as also illustrated in FIG. 5B, nucleic acid fragment sequence data 522 from one or more subjects in a second cohort (e.g., who don't have cancer) can be randomly sampled (532) to select only a subset of all nucleic acid fragment sequences. This sampled subset of nucleic acid fragment sequences can be mixed (540) with the randomly sampled nucleic acid fragment sequences from the one or more subjects in the first cohort, e.g., to generate an augmented set of nucleic acid fragment sequences having a weaker disease signature (e.g., a lower tumor fraction when the disease is cancer) that the original set of nucleic acid fragment sequences from the subject in the first cohort. The mixing of sampled nucleic acid fragment sequences can be used when one of the genomic characteristics used to train a classifier is based on a ratio of disease-derived nucleic acid fragment sequences to healthy nucleic acid fragment sequences. To illustrate, nucleic acid fragment sequence data 520 from a subject in the first cohort (e.g., who has cancer) can be probabilistically sampled (530) using a first probability (e.g., 0.6) to select only a subset of all nucleic acid fragment sequences from the subject. Also, nucleic acid fragment sequence data 522 from a paired subject in a second cohort (e.g., who does not has cancer) can be probabilistically sampled (530) using a second probability (e.g., 0.4), to select only a subset of all nucleic acid fragment sequences from the paired subject. The nucleic acid fragment sequences from the paired subjects, one from cohort 1 and one from cohort 2 can be combined to form a supplemental data construct. More than one subject in cohort 1 and a single subject in cohort 2 can contribute to a single supplemental data construct in this manner. More than one subject in cohort 2 and a single subject in cohort 1 can contribute to a single supplemental data construct in this manner. More than one subject in cohort 2 and more than one subject in cohort 1 can contribute to a single supplemental data construct in this manner. In some embodiments, the nucleic acid fragment sequences in subjects from the first cohort are sampled at a first probability and the nucleic acid fragment sequences in subjects in the second cohort are sampled at a second probability in order to form supplemental data constructs, where the first and second probability are the same or different and where the first and second probability sum or not sum to “1.”

In alternative embodiments, a supplemental data construct is constructed by assigning ‘missing’ nucleic acid fragment sequences as a non-disease state, as opposed to diluting nucleic acid fragment sequences from the diseased cohort with nucleic acid fragment sequences from the non-diseased cohort. For instance, assume that a set nucleic acid fragment sequences from a subject in the first cohort (e.g., a diseased cohort) include 100 nucleic acid fragment sequences for a given genomic locus, of which 20 are derived from a diseased cell. If, by random sampling of 50% of the nucleic acid fragment sequences, 10 nucleic acid fragment sequences derived from diseased cells and 40 nucleic acid fragment sequences derived from healthy cells are selected, the allelic ratio of the augmented set can be 20%, which is the same as the starting sample. However, augmented set of nucleic acid fragment sequences can still include 100 nucleic acid fragment sequences from the locus, in which case the allelic ratio of the augmented set can be determined to be 10% or half of that of the original set of nucleic acid fragment sequences.

As illustrated in FIG. 5B, in some embodiments, the randomly sampled nucleic acid fragment sequences (e.g., generated at step 530 and, optionally, at steps 532 and/or 540) are then used to form (550) the supplemental data constructs of step 514.

In some embodiments, as illustrated in FIG. 5A, workflow 500 includes a step of training a classifier to distinguish between a first disease state associated with the first cohort of subjects and at least a second disease state associated with one or more of the additional cohorts of subjects. As illustrated in FIG. 5A, the training uses data constructs (e.g., which include disease state information about each subject or augmented construct, e.g., disease state information, and genomic characteristics of the biological data obtained or generated for each subject or augmented construct) obtained for subjects in the first cohort, subjects in at least a second cohort, as well as augmented data constructs generated from the randomly sampled nucleic acid fragment sequences from at least one of the subjects in the first cohort.

A method for artificially expanding a data set using probability sampling can generate a plurality of supplemental data constructs (e.g., augmented single time point training constructs and/or augmented time series training constructs) useful for training classifiers to better discriminate different disease states, e.g., for determining whether or not a subject has a disease such as cancer or a cardiovascular disease, for determining a type of disease (e.g., a type of cancer, a primary origin of cancer), for determining a stage of a disease (e.g., a stage of cancer), for determining a prognosis for a disease (e.g., a prognosis for cancer with and/or without treatment), etc.

The method can include obtaining a training dataset (e.g., single time point training data), in electronic form, including a first plurality of genomic data constructs for a first cohort of training subjects (e.g., training subjects) having a first state of the disease condition. The first cohort of training subjects can comprise at least 5, 10, 100, between 10 and 25,000, or less than 100 training subjects.

The first plurality of genomic data constructs can include, for each respective training subject in the first cohort of training subjects, a respective genomic data construct including values for a plurality of genomic characteristics of a corresponding plurality of nucleic acid fragments in a corresponding biological sample obtained from the respective training subject (e.g., corresponding to nucleic acid fragment sequence data). The method can then include using the training dataset to generate the plurality of supplemental data constructs (e.g., augmented single time point training data), where each respective supplemental genomic data construct in the plurality of supplemental genomic data constructs corresponds to (is sampled from) at least a respective genomic data construct from the first plurality of genomic data constructs (e.g., single time point training data).

Each respective supplemental genomic data construct in the plurality of supplemental genomic data constructs can include, for each respective genotypic characteristic in the plurality of genomic characteristics, an augmented value (e.g., one or more of augmented genomic copy number characteristics, augmented variant allele characteristics, augmented allelic ratio characteristics, and augmented genomic methylation characteristics, etc.) that is derived from probability sampling of nucleic acid fragments contributing to the value of the respective genomic characteristic in at least the respective genomic data construct from the first plurality of genomic data constructs. In one example, a supplemental genotypic data construct is formed from the genomic data construct of a training subject in the first cohort by random sampling of each nucleic acid fragment sequence in the training subject. That is, each nucleic acid fragment sequence in the training subject in the first cohort can be accepted into the corresponding supplemental genomic data construct on a probabilistic basis. In this way, the supplemental genomic data construct can achieve an augmented value for each respective genomic characteristic in the plurality of genomic characteristics based upon the identity and characteristics (e.g., one or more of genomic copy number characteristics, variant allele characteristics, allelic ratio characteristics, and genomic methylation characteristics, etc.) of the nucleic acid fragment sequences that are accepted into the corresponding supplemental genomic data construct on the probabilistic basis from the training subject in the first cohort.

The plurality of genomic characteristics can include at least 100, 500, 1000, 5000, 10,000, 50,000, 100,000, or more genotypic characteristics. The plurality of genomic characteristics can include a single type of genotypic characteristic, e.g., one of genomic copy number characteristics, variant allele characteristics, allelic ratio characteristics, and genomic methylation characteristics. In some embodiments, the plurality of genotypic characteristics includes at least two types of genotypic characteristics, e.g., two or more of genomic copy number characteristics, variant allele characteristics, allelic ratio characteristics, and genomic methylation characteristics. The plurality of genotypic characteristics can include at least three types of genotypic characteristics, e.g., three or more of genomic copy number characteristics, variant allele characteristics, allelic ratio characteristics, and genomic methylation characteristics. The values for the plurality of genomic characteristics of the corresponding plurality of nucleic acid fragments can be obtained by whole-genome sequencing, whole-genome methylation sequencing, targeted sequencing (e.g., targeted DNA methylation sequencing) using a plurality of nucleic acid probes to enrich nucleic acids in the corresponding biological sample for a plurality of genomic regions.

More than one single time point training data set can be sampled to form a corresponding supplemental data construct. In this situation, nucleic acid fragment sequences from two or more single time point training data set can be randomly sampled to generate a supplemental data construct representative of an equal amount of, or fewer, nucleic acid fragment sequences than are represented in a single time point training data set. In some embodiments, at least 2 single time point training data sets are sampled together. In other embodiments, at least 3, 4, 5, 6, 7, 8, 9, 10, 25, 50, 100, 500, 1000, or more single time point training data sets are sampled together to form a single supplemental data construct.

In some embodiments, the first plurality of genomic data constructs includes at least a second genomic data construct for one or more training subjects in the first cohort of training subjects, based on a second biological sample obtained from the training subject at a second time. That is, for this training subject, there can be a first genomic data construct obtained using a first biological sample obtained from the training subject at a first time, and a second genomic data construct obtained using a second biological sample obtained from the training subject at a second time. For example, the second sample may be acquired days, weeks, months or years after the first sample. In this situation, there can be more than one genotypic data construct obtained from a training subject using biological samples acquired from the subject over time, where the subject is progressing to later stages of a given cancer over time provide a unique opportunity to augment data. In such embodiments, an augmented genotypic data construct can be constructed by randomly sampling, using a first probability, each nucleic acid fragment in a first genomic data construct acquired at a first time from the subject and randomly sampling, using a second probability, each nucleic acid fragment in a second genotypic data construct acquired at a second time from the subject in order to build an augmented genotypic data construct. Moreover, the first and second probability can be selected such that the distance between the augmented genomic data construct and the first genomic construct and the distance between the augmented genomic data construct and the second genomic construct is controlled. For instance, to obtain an augmented genotypic data construct that is closer to the first genomic construct than the second genomic construct (in terms of genotypic characteristics), each of the nucleic acid fragment sequences of the first genomic construct can be sampled using a higher probability than the probability at which each of the nucleic acid fragment sequences of the second genomic construct are sampled for inclusion in the augmented genomic data construct.

The method can generate a plurality of supplemental data constructs (e.g., augmented time series training constructs) that, when paired with one or more training data constructs, form time series data representative of biological signatures for progression or regression of a disease state (e.g., cancer). The time series data can be useful for training classifiers to better discriminate different disease states, e.g., for determining whether or not a subject has a disease such as cancer or a cardiovascular disease, for determining a type of disease (e.g., a type of cancer, a primary origin of cancer), for determining a stage of a disease (e.g., a stage of cancer), for determining a prognosis for a disease (e.g., a prognosis for cancer with and/or without treatment), etc.

For generating time series data, the method can include obtaining a first training dataset (e.g., time series training data), in electronic form, that includes a first plurality of genomic data constructs for a first cohort of training subjects. The first plurality of genomic data constructs can include, for each respective training subject in the first cohort of training subjects, (i) a respective first genomic data construct comprising values for a plurality of genomic characteristics of a corresponding first plurality of nucleic acid fragments in a corresponding first biological sample obtained from the respective training subject (e.g., corresponding to nucleic acid fragment sequence data) at a respective first time point. The respective training subject can have a first state of the disease condition at the respective first time point (e.g., an absence of a disease, such as cancer or a cardiovascular disease), and (ii) a set of one or more spike-in genomic data constructs for a cohort of one or more spike-in subjects. The set of one or more spike-in genomic data constructs can include a respective spike-in genotypic data construct including values for the plurality of genomic characteristics of a corresponding plurality of nucleic acid fragments in a corresponding biological sample obtained from the respective spike-in subject, where the respective spike-in subject have a second state of the disease condition when the corresponding biological sample is obtained from the respective spike-in subject (e.g., has the disease state, e.g., has cancer). The first state of the disease condition and the second state of the disease condition can be related by progression of the disease condition. For instance, the training subject does not have a disease (e.g., cancer or a cardiovascular disease) or has an early stage of a disease (e.g., stage 0 or stage 1 cancer) and the spike-in subject has the disease and/or has an advanced stage of the disease, such that the sample obtained from the spike-in subject can be treated as a sample from the training subject at a later time, after they have undergone progression of the disease state.

The method can then include using the first training data set to generate a respective first augmented genomic data construct (e.g., augmented time series data) including values for the plurality of genomic characteristics that are representative of the respective training subject at a respective second time point. The respective first augmented genomic data construct can correspond to a corresponding first pair of genomic data constructs. The first pair of genomic data constructs can comprise (i) a respective second genomic data construct for the respective training subject and (ii) a respective spike-in genomic data construct from the set of one or more spike-in genotypic data constructs. The respective first augmented genomic data construct can include an augmented value that is derived from a first probability sampling of nucleic acid fragments contributing to the value of the respective genomic characteristic in each genomic data construct of the corresponding first pair of genotypic data constructs. The method can thereby generate, for each respective training subject in the first cohort of training subjects, a respective time series data set including the respective first genomic data construct and the respective first augmented genomic data construct. Details of genomic characteristics and disease conditions are described elsewhere herein.

For at least one respective training subject in the first cohort of training subjects, the respective training subject may not be afflicted with cancer at the respective first time point. The respective spike-in genotypic data construct in the pair of genotypic data constructs may be obtained from a corresponding spike-in subject who is afflicted with at least stage 2 cancer when the corresponding biological sample was obtained from the respective spike-in subject.

A respective time-series data set, including the respective first genotypic data construct and the respective first augmented genotypic data construct, can be generated for each respective training subject in the first cohort of training subjects. Accordingly, the respective first augmented genomic data construct can correspond to a corresponding first pair of genomic data constructs. The first pair of genomic data constructs can comprise (i) a respective second genomic data construct for the respective training subject and (ii) a respective spike-in genomic data construct from the set of one or more spike-in genomic data constructs.

The spike-in subject can be a different subject than the training subject, e.g., in a case where the samples from the training subject and the spike-in subject are collected contemporaneously or the training subject never develops the disease state. In such a case, disease signal from the spike-in sample can be mixed directly with the first sample obtained from the training subject to form a data construct corresponding to the second time point for the training subject. Accordingly, for at least one respective training subject in the first cohort of training subjects, the respective second genomic data construct can be the respective first genomic data construct. However, a second sample can also be obtained from the training subject, and used as the background for the data construct corresponding to the second time point, e.g., when the training subject does not subsequently develop the disease or does not experience substantial progression of the disease. Disease signal from the spike-in sample can be mixed with background from the second sample from the training subject to form a data construct corresponding to the second time point for the training subject. Accordingly, for at least one respective training subject in the first cohort of training subjects, the respective second genomic data construct can include values for the plurality of genomic characteristics of a corresponding second plurality of nucleic acid fragments in a corresponding second biological sample obtained from the respective training subject at the second time point. The spike-in subject corresponding to the respective spike-in genotypic data construct in the corresponding pair of genotypic data constructs can be matched to the respective training subject based on a shared personal characteristic, e.g., to account for variation associated with factors other than disease progression.

In one example, device 100 randomly samples (530) nucleic acid fragment sequence data from one or more training constructs (e.g., 520) and one or more spike-in samples (e.g., 522) select subsets of nucleic acid fragment sequences (e.g., augmented nucleic acid fragment sequence data 152-n), which are used to construct a supplemental data construct (550). The mixing can be thought of as diluting the biological disease signal from the spike-in sample with background from the training data construct, to generate a data construct representative of the training subject at a second time after they have experienced progression of the disease state. The nucleic acid fragment sequence data from the one or more training constructs can be sampled using simple random sampling with a first probability, and the nucleic acid fragment sequence data from the one or more spike-in samples can be sampled using simple random sampling with a second probability, where the first probability is the same or different. In some embodiments, the first and second probability are the same. The first probability can be at least 5%, 10%, 15%, 20%, 30%, 40%, 50%, 60%, 70%, 80%, 90% and more. The first probability can be at most 90%, 80%, 70%, 60%, 50%, 40%, 30%, 20%, 10% or less. The second probability can be at least 5%, 10%, 15%, 20%, 30%, 40%, 50%, 60%, 70%, 80%, 90% and more. The second probability can be at most 90%, 80%, 70%, 60%, 50%, 40%, 30%, 20%, 10% or less. The first probability can be the same as or different from the second probability.

Additional augmented time points can also be generated, e.g., by mixing different amounts of the biological signal from the spike-in sample with biological signal from the training samples, or by mixing biological signal from a series of spike-in samples representing a time course for disease progression or regression. In some embodiments, the time-series data includes at least 3 time points, or at least 4, 5, 6, 7, 8, 9, 10, or more time points.

Mixing of the biological signals between the training sample and the spike-in sample can be informed by a model of disease progression. For instance, a cancer progression model is used to determine how much additional cancer signal (e.g., provided by the spike-in sample) can be added to the training sample at each time point to replicate a given progression of the cancer. Accordingly, the probability sampling can select a respective first portion of the plurality of nucleic acid fragments that contribute to the values for the plurality of genomic characteristics in the first respective genomic construct and a respective second portion of the plurality of nucleic acid fragments that contribute to the values for the plurality of genomic characteristics in the respective spike-in genomic data construct. The magnitude of the respective first portion of nucleic acid fragments and the respective second portion of nucleic acid fragments can be determined based on at least (i) the length of time between the first time point and the second time point and (ii) a temporal model for development of the second state of the disease condition from the first state of the disease condition.

The modeling of disease progression or regression can consider one or more personal characteristics of the subject. For example, lung cancer can progress more quickly in a subject that smokes than in a subject that does not smoke. According, the temporal model for development of the second state of the disease condition from the first state of the disease condition is based at least on a personal characteristic of the respective subject, e.g., one or more of gender, age, family medical history, personal medical history, ethnicity, smoking status, alcohol consumption status, anthropomorphic data, etc. The modeling of disease progression or regression can be specific for a particular form of the disease, e.g., cancer. For instance, the disease condition is cancer, and the temporal model for development of the second state of the cancer from the first state of the cancer is based at least on the type of cancer. In another example, the disease condition can be cancer, and the temporal model for development of the second state of the cancer from the first state of the cancer can be based at least on whether the cancer is metastatic or non-metastatic. In yet another example, the disease condition is cancer, and the temporal model for development of the second state of the cancer from the first state of the cancer is separated into stages.

In some embodiments, each nucleic acid fragment sequence can be sampled on a probabilistic basis for inclusion in the supplemental data construct. Each nucleic acid fragment sequence can be sampled on a probabilistic basis for inclusion in the supplemental data construct, where the probability of inclusion is the same (e.g., between 5 percent and 95 percent, five percent, 10 percent, 15 percent, 20 percent, 25 percent, 30 percent, 35 percent, 40 percent, 45 percent, 50 percent, 55 percent, 60 percent, 65 percent, 70 percent, 75 percent, 80 percent, 85 percent, 90 percent) for each nucleic acid fragment sequence. In some embodiments of random sampling, each nucleic acid fragment sequence can be sampled on a probabilistic basis for inclusion in the supplemental data construct, where the probability of inclusion is dependent upon which bin, in a plurality of bins, the nucleic acid fragment sequence corresponds to, where each bin in the plurality of bins represents a different portion of a reference genome. The actual probability value that is used can be application dependent (e.g., based on the detection limits of a trained classifier). In one example, the detection limit of a classifier can be gauged by the metric of tumor fraction, and an augmented dataset that represents tumor fractions at the limit of detection of a classifier can be generated using the disclosed systems and methods.

The disease condition can be cancer. For instance, the first state of the cancer is a presence of the cancer and a second state of the cancer is an absence of the cancer. In this situation, a classifier can be trained against features from a first cohort of patients that have cancer, features from a second cohort of patients that do not have cancer, and simulated features from a set of augmented data constructs, e.g., with cancer signals that are generally weaker than those of the first cohort. The first state of the cancer can be a first type of cancer and a second state of the cancer can be a second type of cancer. In this situation, a classifier can be trained against features from a first cohort of patients having a first type of cancer, features from a second cohort of patients having a second type of cancer, and simulated features from a set of augmented data constructs, e.g., with cancer signals for the first and/or second type of cancer that are generally weaker than those of the first and/or second cohort. The first state of the cancer can be a first stage of a specified cancer and a second state of the cancer can be a second stage of the specified cancer. In this situation, a classifier can be trained to distinguish between different stages of the same or different types of cancer, e.g., between two or more of stage 0, stage 1, stage 2, stage 3, and stage 4 cancer. The first state of the cancer can be a first prognosis for the cancer and a second state of the cancer can be a second prognosis for the cancer. In this situation, a classifier can be trained to distinguish between different life expectancies without treatment, different life expectancies with treatment, different expected remission rates, and/or different expected responses to a particular therapy.

In some embodiments, the disease condition is a cardiovascular disease. The first state of the cardiovascular disease can be a presence of the cardiovascular disease and a second state of the cardiovascular disease can be an absence of the cardiovascular disease. In this situation, a classifier can be trained against features from a first cohort of patients that have a cardiovascular disease, features from a second cohort of patients that do not have the cardiovascular disease, and simulated features from a set of augmented data constructs, e.g., with cardiovascular disease signals that are generally weaker than those of the first cohort. The first state of the cardiovascular disease can be a first prognosis for the cardiovascular disease and a second state of the cardiovascular disease can be a second prognosis for the cardiovascular disease. In this situation, a classifier can be trained to distinguish between different life expectancies without treatment, different life expectancies with treatment, different expected remission rates, and/or different expected responses to a particular therapy.

In some embodiments, biological data from one or more data constructs in a second cohort 522, can also be randomly sampled and mixed with the randomly sampled data from the first data construct, to form a supplemental data construct from the combination of nucleic acid fragments probabilistically sampled from one or more subjects in the first cohort and nucleic acid fragments probabilistically sampled from one or more subjects in the second cohort. The biological data from a single data construct in a second cohort 522 can also be randomly sampled and mixed with the randomly sampled data from the first data construct, to form a supplemental data construct from the combination of nucleic acid fragments probabilistically sampled from a single subject in the first cohort and nucleic acid fragments probabilistically sampled from a single subject in the second cohort. Where the first data construct corresponds to a subject having a particular disease state (e.g., having cancer or having a cardiovascular disease) and the second data construct corresponds to a subject that does not have the particular disease state (e.g., doesn't have cancer or doesn't have the cardiovascular disease), the mixing can be thought of as diluting the biological disease signals from the first data construct with background from the second data construct. Accordingly, the training data set can further include a second plurality of genomic data constructs for a second cohort of training subjects having a second state of the disease condition that is different from the first state of the disease condition. The second plurality of genomic data constructs can include a respective genotypic data construct including values for the plurality of genomic characteristics of a corresponding plurality of nucleic acid fragments in a corresponding biological sample obtained from the respective training subject. The sampled data may not be diluted from the first data construct. Training a classifier with augmented data constructs in which the sampled data is not mixed with a background signal can generate a better classifier than is generated when a model is trained using the same sampled signal mixed with background signal.

The methods for randomly sampling data can include simple random sampling, stratified random sampling, systematic random sampling, clustered random sampling, and multi-stage random sampling. Simple random sampling may include that each item in a group (here, each nucleic acid fragment sequence in a subject, or a plurality of subjects, in one or more training cohorts) has the same probability of being chosen. For example, simple random sampling of a set of nucleic acid fragment sequences dictates that each nucleic acid fragment sequence in the set has a set chance of being selected for the set of augmented nucleic acid fragment sequences. A combination of stratified sampling or cluster sampling and simple random sampling can be employed. Various considerations may dictate what selection probability is used for any particular sampling event. These considerations can include, but are not limited to, the amount of disease signals in the starting data construct (e.g., the tumor fraction and/or mutational burden for a data construct corresponding to a cancer patient), the amount of disease signals desired in the supplemental data construct, and the amount of disease signals in other training data constructs.

The probability sampling can include weighted random sampling of a predetermined portion of the plurality of nucleic acid fragments contributing to the values of the plurality of genomic characteristics, where the probability of selecting a respective nucleic acid fragment that contributes to the value of a corresponding genomic characteristic is proportional to the abundance of nucleic acid fragments contributing the corresponding genomic characteristic relative to the total number of nucleic acid fragments contributing to the values of the plurality of genotypic characteristics. The probability sampling can select a respective portion of the plurality of nucleic acid fragments that contribute to the values for the plurality of genomic characteristics in the respective data construct from the first plurality of genomic data constructs. The magnitude of the respective portion of nucleic acid fragments can be determined independently from the magnitudes of the respective portions of nucleic acid fragments selected for the other supplemental data constructs. The methodology used to sample different training data sets can be selected independently, e.g., to account for factors such as the amount of disease signals in each data construct. The magnitude of the respective portion of nucleic acid fragments can be selected such that the respective supplemental data construct represents a simulated informative nucleic acid fragment fraction falling within a range of informative nucleic acid fragment fractions over which an exploratory classifier satisfies a threshold sensitivity to changes in the informative nucleic acid fragment fraction represented by the genotypic data construct, where the exploratory classifier is trained to discriminate a state of the disease condition based on the plurality of genotypic characteristics. Supplemental data constructs can be formed such that their disease signals (e.g., tumor fraction in the case of a cancer patient) fall within a range around the predicted level of detection (LOD) for the classifier.

The range of informative nucleic acid fragment fractions (e.g., tumor fraction) can be determined by using the training dataset to generate a plurality of augmented exploratory genomic data constructs. Each respective augmented exploratory genomic data construct in the plurality of augmented exploratory genomic data constructs can correspond to at least a respective genomic data construct from the first plurality of genomic data constructs. Each respective augmented exploratory genomic data construct in the plurality of augmented exploratory genotypic data constructs can include an augmented value that is derived from probability sampling of nucleic acid fragments contributing to the value of the respective genomic characteristic from at least the respective genomic data construct from the first plurality of genomic data constructs. Each respective augmented exploratory genomic data construct in the plurality of augmented exploratory genomic data constructs can represent a simulated informative nucleic acid fragment fraction that is based upon the informative nucleic acid fragment fraction represented by the respective genomic data construct from the first plurality of genomic data constructs. The distribution of simulated informative nucleic acid fragment fractions represented by the plurality of augmented exploratory genomic data constructs can span from a first informative nucleic acid fragment fraction that is below a level of detection for an exploratory classifier to a second informative nucleic acid fragment fraction that is above the level of detection for the exploratory classifier. The distribution of simulated informative nucleic acid fragment fractions can span from about 1% above the level of detection to about 1% below the level of detection, from about 2% above the level of detection to about 2% below the level of detection, from about 5% above the level of detection to about 5% below the level of detection, from about 10% above the level of detection to about 10% below the level of detection, from about 15% above the level of detection to about 15% below the level of detection, or from about 20% above the level of detection to about 20% below the level of detection.

The range of informative nucleic acid fragment fractions can be determined by training a preliminary classifier, e.g., of the same type as the ultimate classifier, using all or a subset of the single time point training data. Then, a plurality of augmented exploratory genotypic data constructs can be applied to the exploratory classifier to generate a plurality of simulated disease condition probabilities. The exploratory classifier can be trained to discriminate a state of the disease condition using at least: (1) a first plurality of exploratory genomic data constructs, where the first plurality of exploratory genotypic data constructs includes a respective genomic data construct including values for the plurality of genomic characteristics of a corresponding plurality of nucleic acid fragments in a corresponding biological sample obtained from the respective exploratory subject, (2) a second plurality of exploratory data constructs, where the second plurality of exploratory genotypic data constructs includes a respective genomic data construct including values for the plurality of genomic characteristics of a corresponding plurality of nucleic acid fragments in a corresponding biological sample obtained from the respective exploratory subject, and (3) for each respective genomic data construct in the first and second pluralities of exploratory genomic data constructs, an indication of the state of the disease condition. The range of informative nucleic acid fragment fractions can be identified over which simulated disease condition probabilities are most sensitive to changes in the informative nucleic acid fragment fraction represented by a respective augmented exploratory genotypic data construct.

Methods for extracting genomic features/characteristics from a plurality of electronic sequence can be found in, for instance, U.S. Patent Application Publication No. 2019/0287652, the content of which is incorporated herein by reference for all purposes, describes methods for determining the methylation status of a plurality of genomic locations. Similarly, U.S. Patent Application Publication No. 2019/0287649, the content of which is incorporated herein by reference for all purposes, describes methods for determining the relative copy number of a plurality of genomic locations.

The genomic characteristics can comprise a plurality of relative copy numbers (e.g., bin read counts), where each respective relative copy number in the plurality of relative copy numbers corresponds to a different genetic location in a plurality of genetic locations. The relative copy numbers can represent the relative abundance of sequence reads from a plurality of genomic regions. The genomic regions can have the same or different size. A genomic region can be defined by the number of nucleic acid residues within the region or its location and the number of nucleic acid residues within the region. For example, a genomic region can include 10 kb or fewer, 20 kb or fewer, 30 kb or fewer, 40 kb or fewer, 50 kb or fewer, 60 kb or fewer, 70 kb or fewer, 80 kb or fewer, 90 kb or fewer, 100 kb or fewer, 110 kb or fewer, 120 kb or fewer, 130 kb or fewer, 140 kb or fewer, 150 kb or fewer, 160 kb or fewer, 170 kb or fewer, 180 kb or fewer, 190 kb or fewer, x200 kb or fewer, or 250 kb or fewer. The genomic regions can be defined by dividing a reference genome for the species of the subject into a plurality of segments (i.e., the genomic regions). For instance, a reference genome is divided into up to 1,000 regions, 2,000 regions, 4,000 regions, 6,000 regions, 8,000 regions, 10,000 regions, 12,000 regions, 14,000 regions, 16,000 regions, 18,000 regions, 20,000 regions, 22,000 regions, 24,000 regions, 26,000 regions, 28,000 regions, 30,000 regions, 32,000 regions, 34,000 regions, 36,000 regions, 38,000 regions, 40,000 regions, 42,000 regions, 44,000 regions, 46,000 regions, 48,000 regions, 50,000 regions, 55,000 regions, 60,000 regions, 65,000 regions, 70,000 regions, 80,000 regions, 90,000 regions, or up to 100,000 regions. Sequence reads of a subject can be normalized to the average read count across all chromosomal regions for the subject, e.g., as described in U.S. Patent Application Publication No. 2019/0287649, the content of which is incorporated herein by reference. The copy number data can be further normalized, e.g., to reduce or eliminate variance in the sequencing data caused by potential confounding factors. The normalizing can involve one or more of centering on a measure of central tendency within the sample, centering on data from a reference sample or cohort, normalization for GC content, and principal component analysis (PCA) correction. Additionally or alternatively, the normalization may include B-score processing, as described in U.S. Patent Application Publication No. 2019/0287649.

The plurality of genomic characteristics can include a plurality of methylation statuses (e.g., regional methylation statuses), where each methylation status in the plurality of methylation statuses corresponds to a different genetic location in a plurality of genetic locations. In some embodiments, each methylation status is represented by a methylation state vector as described, for example, in U.S. Patent Application Publication No. 2019/0287652, which is hereby incorporated by reference herein in its entirety. The plurality of methylation statuses can be obtained by a targeted DNA methylation sequencing using a plurality of probes. The plurality of probes can hybridize to at least 100 loci in the human genome. In other embodiments, the plurality of probes hybridize to at least 250, 500, 750, 1000, 2500, 5000, 10,000, 25,000, 50,000, 100,000, or more loci in the human genome. Methods for identifying informative methylation loci for classifying a disease condition (e.g., cancer) are described, for instance, in U.S. Patent Application Publication No. 2019/0287649. Methylation data can be normalized, e.g., to reduce or eliminate variance in the sequencing data caused by potential confounding factors. In some embodiments, the normalizing involves one or more of centering on a measure of central tendency within the sample, centering on data from a reference sample or cohort, normalization for GC content, and principal component analysis (PCA) correction. Further description of normalization of methylation data can be found, for example, in U.S. Patent Application Publication No. 2019/0287652 and U.S. Patent Application Publication No. 2019/0287649, the disclosures of both which are incorporated herein by reference.

The plurality of genomic characteristics in a genomic data construct (e.g., a training, augmented, and/or test genotypic data construct) can include a first plurality of bin values (e.g., regional methylation statuses). Each respective bin value in the first plurality of bin values can represent a corresponding bin in a plurality of bins. Each respective bin value in the first plurality of bin values can be representative of a number of unique nucleic acid fragments with a predetermined methylation pattern identified from a corresponding set of nucleic acid fragment sequences (e.g., a training set, augmented set, or test set) that map to the corresponding bin in the plurality of bins. Each bin in the plurality of bins can represent a non-overlapping region of a reference genome of a species of the subject.

III.C. Training of Cancer Classifier

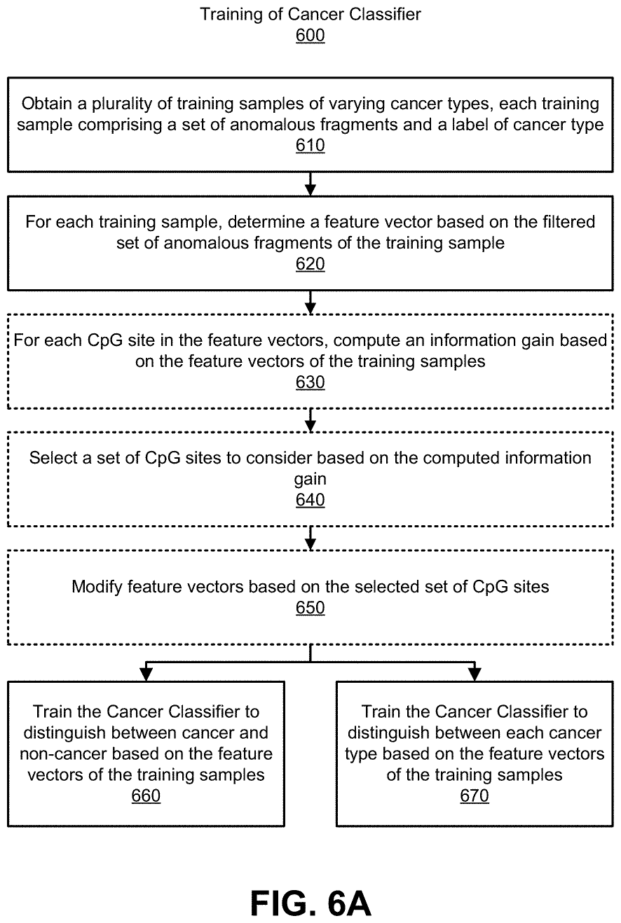

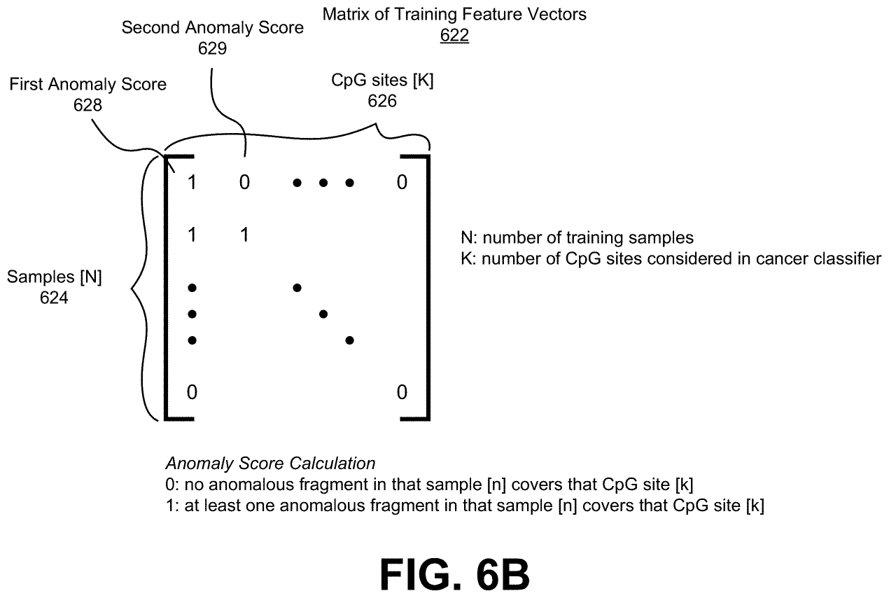

FIG. 6A is a flowchart describing a process 600 of training a cancer classifier, according to an embodiment. The analytics system obtains 510 a plurality of training samples each having a set of anomalous fragments and a label of a cancer type. The plurality of training samples can include any combination of samples from healthy individuals with a general label of “non-cancer,” samples from subjects with a general label of “cancer” or a specific label (e.g., “breast cancer,” “lung cancer,” etc.). The training samples from subjects for one cancer type may be termed a cohort for that cancer type or a cancer type cohort.

The analytics system determines 520, for each training sample, a feature vector based on the set of anomalous fragments of the training sample. The analytics system can calculate an anomaly score for each CpG site in an initial set of CpG sites. The initial set of CpG sites may be all CpG sites in the human genome or some portion thereof—which may be on the order of 10⁴, 10⁵, 10⁶, 10⁷, 10⁸, etc. In one embodiment, the analytics system defines the anomaly score for the feature vector with a binary scoring based on whether there is an anomalous fragment in the set of anomalous fragments that encompasses the CpG site. In another embodiment, the analytics system defines the anomaly score based on a count of anomalous fragments overlapping the CpG site. In one example, the analytics system may use a trinary scoring assigning a first score for lack of presence of anomalous fragments, a second score for presence of a few anomalous fragments, and a third score for presence of more than a few anomalous fragments. For example, the analytics system counts 5 anomalous fragment in a sample that overlap the CpG site and calculates an anomaly score based on the count of 5.

Once all anomaly scores are determined for a training sample, the analytics system can determine the feature vector as a vector of elements including, for each element, one of the anomaly scores associated with one of the CpG sites in an initial set. The analytics system can normalize the anomaly scores of the feature vector based on a coverage of the sample. Here, coverage can refer to a median or average sequencing depth over all CpG sites covered by the initial set of CpG sites used in the classifier, or based on the set of anomalous fragments for a given training sample.

As an example, reference is now made to FIG. 6B illustrating a matrix of training feature vectors 622. In this example, the analytics system has identified CpG sites [K] 626 for consideration in generating feature vectors for the cancer classifier. The analytics system selects training samples [N] 624. The analytics system determines a first anomaly score 628 for a first arbitrary CpG site [k1] to be used in the feature vector for a training sample [n1]. The analytics system checks each anomalous fragment in the set of anomalous fragments. If the analytics system identifies at least one anomalous fragment that includes the first CpG site, then the analytics system determines the first anomaly score 628 for the first CpG site as 1, as illustrated in FIG. 6B. Considering a second arbitrary CpG site [k2], the analytics system similarly checks the set of anomalous fragments for at least one that includes the second CpG site [k2]. If the analytics system does not find any such anomalous fragment that includes the second CpG site, the analytics system determines a second anomaly score 629 for the second CpG site [k2] to be 0, as illustrated in FIG. 6B. Once the analytics system determines all the anomaly scores for the initial set of CpG sites, the analytics system determines the feature vector for the first training sample [n1] including the anomaly scores with the feature vector including the first anomaly score 628 of 1 for the first CpG site [k1] and the second anomaly score 629 of 0 for the second CpG site [k2] and subsequent anomaly scores, thus forming a feature vector [1, 0, . . . ].

Additional approaches to featurization of a sample can be found in: U.S. application Ser. No. 15/931,022 entitled “Model-Based Featurization and Classification;” U.S. application Ser. No. 16/579,805 entitled “Mixture Model for Targeted Sequencing;” U.S. application Ser. No. 16/352,602 entitled “Anomalous Fragment Detection and Classification;” and U.S. application Ser. No. 16/723,716 entitled “Source of Origin Deconvolution Based on Methylation Fragments in Cell-Free DNA Samples;” all of which are incorporated by reference in their entirety.

The analytics system may further limit the CpG sites considered for use in the cancer classifier. The analytics system computes 530, for each CpG site in the initial set of CpG sites, an information gain based on the feature vectors of the training samples. From step 520, each training sample has a feature vector that may contain an anomaly score all CpG sites in the initial set of CpG sites which could include up to all CpG sites in the human genome. However, some CpG sites in the initial set of CpG sites may not be as informative as others in distinguishing between cancer types, or may be duplicative with other CpG sites.

In one embodiment, the analytics system computes 530 an information gain for each cancer type and for each CpG site in the initial set to determine whether to include that CpG site in the classifier. The information gain is computed for training samples with a given cancer type compared to all other samples. For example, two random variables ‘anomalous fragment’ (‘AF’) and ‘cancer type’ (‘CT’) are used. In one embodiment, AF is a binary variable indicating whether there is an anomalous fragment overlapping a given CpG site in a given samples as determined for the anomaly score/feature vector above. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. In practice, for a first cancer type, the analytics system computes pairwise mutual information gain against each other cancer type and sums the mutual information gain across all the other cancer types.

For a given cancer type, the analytics system can use this information to rank CpG sites based on how cancer specific they are. This procedure can be repeated for all cancer types under consideration. If a particular region is commonly anomalously methylated in training samples of a given cancer but not in training samples of other cancer types or in healthy training samples, then CpG sites overlapped by those anomalous fragments can have high information gains for the given cancer type. The ranked CpG sites for each cancer type can be greedily added (selected) 540 to a selected set of CpG sites based on their rank for use in the cancer classifier.

In additional embodiments, the analytics system may consider other selection criteria for selecting informative CpG sites to be used in the cancer classifier. One selection criterion may be that the selected CpG sites are above a threshold separation from other selected CpG sites. For example, the selected CpG sites are to be over a threshold number of base pairs away from any other selected CpG site (e.g., 100 base pairs), such that CpG sites that are within the threshold separation are not both selected for consideration in the cancer classifier.

In one embodiment, according to the selected set of CpG sites from the initial set, the analytics system may modify 550 the feature vectors of the training samples as needed. For example, the analytics system may truncate feature vectors to remove anomaly scores corresponding to CpG sites not in the selected set of CpG sites.

With the feature vectors of the training samples, the analytics system may train the cancer classifier in any of a number of ways. The feature vectors may correspond to the initial set of CpG sites from step 520 or to the selected set of CpG sites from step 550. In one embodiment, the analytics system trains 560 a binary cancer classifier to distinguish between cancer and non-cancer based on the feature vectors of the training samples. In this manner, the analytics system uses training samples that include both non-cancer samples from healthy individuals and cancer samples from subjects. Each training sample can have one of the two labels “cancer” or “non-cancer.” In this embodiment, the classifier outputs a cancer prediction indicating the likelihood of the presence or absence of cancer.

In another embodiment, the analytics system trains 450 a multiclass cancer classifier to distinguish between many cancer types (also referred to as tissue of origin (TOO) labels). Cancer types can include one or more cancers and may include a non-cancer type (may also include any additional other diseases or genetic disorders, etc.). To do so, the analytics system can use the cancer type cohorts and may also include or not include a non-cancer type cohort. In this multi-cancer embodiment, the cancer classifier is trained to determine a cancer prediction (or, more specifically, a TOO prediction) that comprises a prediction value for each of the cancer types being classified for. The prediction values may correspond to a likelihood that a given training sample (and during inference, a test sample) has each of the cancer types. In one implementation, the prediction values are scored between 0 and 100, wherein the cumulation of the prediction values equals 100. For example, the cancer classifier returns a cancer prediction including a prediction value for breast cancer, lung cancer, and non-cancer. For example, the classifier can return a cancer prediction that a test sample is 65% likelihood of breast cancer, 25% likelihood of lung cancer, and 10% likelihood of non-cancer. The analytics system may further evaluate the prediction values to generate a prediction of a presence of one or more cancers in the sample, also may be referred to as a TOO prediction indicating one or more TOO labels, e.g., a first TOO label with the highest prediction value, a second TOO label with the second highest prediction value, etc. Continuing with the example above and given the percentages, in this example the system may determine that the sample has breast cancer given that breast cancer has the highest likelihood.

In both embodiments, the analytics system trains the cancer classifier by inputting sets of training samples with their feature vectors into the cancer classifier and adjusting classification parameters so that a function of the classifier accurately relates the training feature vectors to their corresponding label. The analytics system may group the training samples into sets of one or more training samples for iterative batch training of the cancer classifier. After inputting all sets of training samples including their training feature vectors and adjusting the classification parameters, the cancer classifier can be sufficiently trained to label test samples according to their feature vector within some margin of error. The analytics system may train the cancer classifier according to any one of a number of methods. As an example, the binary cancer classifier may be a L2-regularized logistic regression classifier that is trained using a log-loss function. As another example, the multi-cancer classifier may be a multinomial logistic regression. In practice either type of cancer classifier may be trained using other techniques. These techniques are numerous including potential use of kernel methods, random forest classifier, a mixture model, an autoencoder model, machine learning algorithms such as multilayer neural networks, etc.

In some embodiments, the supplemental data constructs can be used (e.g., in conjunction with the original cohort data from which the supplemental data constructs are derived by random sampling) to train a classifier to distinguish between two or more disease states. The training data set can further include a second plurality of genomic data constructs for a second cohort of training subjects having a second state of the disease condition that is different from the first state of the disease condition. The second plurality of genomic data constructs can include a respective genomic data construct including values for the plurality of genomic characteristics of a corresponding plurality of nucleic acid fragments in a corresponding biological sample obtained from the respective training subject. The method can include a step of training a classifier to discriminate a state of the disease condition using at least (i) the first plurality of genomic data constructs, (ii) a second plurality of genomic data constructs, (iii) the plurality of supplemental genomic data constructs, and (iv) for each respective genomic data construct in the first plurality of genotypic data constructs, second plurality of genomic data constructs, and the plurality of supplemental genomic data constructs, an indication of the state of the disease condition.

The training can additionally use a third plurality of genotypic data constructs for a third cohort of training subjects. The third plurality of genomic data constructs can include a respective genomic data construct including values for the plurality of genotypic characteristics of a corresponding plurality of nucleic acid fragments in a corresponding biological sample obtained from the respective training subject. Each training subject in the third cohort can have a third state of the disease condition. In this fashion, a classifier can be trained to distinguish between the first, second, and third disease states. The training can additionally use one or more personal characteristics of respective training subjects. For example, one or more of gender, age, family medical history, personal medical history, ethnicity, smoking status, alcohol consumption status, anthropomorphic data, etc., are used.

One or more of the supplemental genomic data constructs can be formed from a mixture of randomly sampled biological characteristics (e.g., nucleic acid fragment sequences) from data constructs from different cohorts, e.g., a diseased cohort and a healthy cohort. Each respective supplemental genomic data construct in the plurality of supplemental genomic data constructs can correspond to a corresponding pair of genomic data constructs. The pair of genomic data constructs can comprise (i) the respective genomic data construct from the first plurality of genomic data constructs (e.g., corresponding to a diseased subject) and (ii) a respective genomic data construct from the second plurality of genomic data constructs (e.g., corresponding to a healthy subject). Each respective supplemental genomic data construct in the supplemental plurality of genomic data constructs can include an augmented value that is derived from a probability sampling of nucleic acid fragments contributing to the value of the respective genomic characteristic in each genomic data construct of the corresponding pair of genomic data constructs.

For at least one respective supplemental genomic data construct in the plurality of supplemental genotypic data constructs, the respective genomic data construct from the second plurality of genotypic data constructs can be augmented prior to deriving the augmented values for the plurality of genotypic characteristics of the respective supplemental genotypic data construct. The augmented value for each respective genomic characteristic in the plurality of genomic characteristics can be formed from (i) a first weighted contribution of the respective genomic characteristic from the respective genomic data construct from the first plurality of genomic data constructs, and (ii) a second weighted contribution of the respective genomic characteristic from the respective genomic data construct from the second plurality of genotypic data constructs. In this fashion, an informative nucleic acid fraction (e.g., tumor fraction when considering cancer) can be obtained in the supplemental data construct by controlling the proportion of disease signals contributed from each original data set.

When mixing biological information derived from subjects in the same or different cohorts, the data constructs can be selected by matching one or more personal characteristics of the subjects corresponding to the data constructs, e.g., to account for biological variance introduced by such personal characteristics. For each respective supplemental genomic data construct in the plurality of supplemental genomic data constructs, the (i) respective training subject corresponding to the respective genomic data construct from the first plurality of genomic data constructs and (ii) respective training subject corresponding to the respective genomic data construct from the second plurality of genomic data constructs, corresponding to the pair of genomic data constructs, can be matched based on a shared personal characteristic.

Artificially generated time-series data set can be used to train a classifier to distinguish between two or more disease states. Accordingly, training a temporal classifier to discriminate a state of the disease condition can use at least (i) for each respective training subject in the first cohort of training subjects, the respective time-series data set, (ii) for each respective training subject in the first cohort of training subjects, a respective plurality of time points including a respective time point for each respective genomic data construct in the respective time-series data set, or a derivation thereof, and (iii) for each respective training subject in the first cohort of training subjects, an indication of the disease condition for at least the earliest respective time point and the latest respective time point in the respective plurality of time points. The training can use one or more personal characteristics of the respective training subject. For example, one or more of gender, age, family medical history, personal medical history, ethnicity, smoking status, alcohol consumption status, anthropomorphic data, etc. Details of the classifier are described elsewhere herein.

The method for training a temporal classifier using artificially created data representing a time-series simulating cancer progression can include obtaining a training dataset (e.g., time-series training data), in electronic form, that includes, for each respective training subject in a plurality of training subjects: (1) a respective first genomic data construct for the respective training subject, the respective first genomic data construct including values for a plurality of genomic characteristics of a first respective plurality of nucleic acid fragments in a first biological sample obtained from the respective training subject at a respective first time point (e.g., time-series training data point), (2) a respective second genomic data construct for the respective training subject, the respective second genomic data construct including values for the plurality of genomic characteristics that are representative of the respective training subject at a respective second time point occurring after the respective first time point (e.g., augmented time-series data point), (3) the respective first time point and the respective second time point, or a derivation thereof (e.g., a time which the first and second data points correspond to or the amount of time between the two time points), and (4) an indication of the disease condition in the set of disease conditions, at the respective first time and the respective second time point, of the respective training subject.

The method can then include training a temporal classification algorithm against, for each respective training subject, at least (a) the respective first genomic data construct, (b) the respective second genomic data construct, (c) the respective first time point and the respective second time point, or the derivation thereof, and (d) the indication of the disease condition, at the respective first time and the respective second time point. For at least one respective training subject in the plurality of training subjects, the respective second genomic data construct can include values for the plurality of genomic characteristics from a respective second plurality of nucleic acid fragments from a second biological sample obtained from the respective training subject and a respective third plurality of nucleic acid fragments from a spike-in biological sample obtained from a spike-in subject afflicted with a respective state of the disease condition in the set of states of the disease condition.

The respective second genomic data construct can include, for each respective genomic characteristic in the plurality of genomic characteristics, an augmented value that is derived from a probability sampling of (i) nucleic acid fragments contributing to the value of the respective genomic characteristic in the second plurality of nucleic acid fragments, and (ii) nucleic acid fragments contributing to the value of the respective genomic characteristic in the third plurality of nucleic acid fragments. The sampling can be thought of as diluting the biological disease signal from the spike-in sample with background from the training data construct, to generate a data construct representative of the training subject at a second time after they have experienced progression of the disease state.

The respective third genomic data construct can include values for the plurality of genomic characteristics that are representative of the respective training subject at a respective third time point occurring after the respective second time point, the respective third time point, or a derivation of the respective second time point and the respective third time point (e.g., the period of time between points), and an indication of the state of the disease condition in the set of states of the disease condition, at the respective third time point, of the respective training subject. For at least one respective training subject in the plurality of training subjects, the respective third genomic data construct can include values for the plurality of genomic characteristics from a respective fourth plurality of nucleic acid fragments from a third biological sample obtained from the respective training subject and a respective fifth plurality of nucleic acid fragments from a spike-in biological sample obtained from a spike-in subject with the respective state of the disease condition in the set of states of the disease condition.

The respective second plurality of nucleic acid fragments and the respective fourth plurality of nucleic acid fragments can be the same cell-free nucleic acids from the same biological sample obtained from the respective training subject. In this situation, the same background sample from the training subject used to form the second genomic data construct can be used to form the third genotypic data construct, e.g., by mixing with biological signal from a different spike-in sample or a different amount of biological signal from the same spike-in sample.

The respective third plurality of nucleic acid fragments and the respective fifth plurality of nucleic acid fragments can be the same cell-free nucleic acids from the same spike-in biological sample obtained from the spike-in subject. In this situation, the same spike-in sample from the spike-in subject used to form the second genotypic data construct can be used to form the third genotypic data construct, e.g., by mixing in a different proportion with biological signal from a background sample, which might be the same or different background sample as used to construct the second genotypic data construct. The values for the plurality of genomic characteristics in the respective second genotypic data construct can include a respective first weighted mixture of (i) values for the plurality of genomic characteristics of the respective second plurality of nucleic acid fragments and (ii) values for the plurality of genomic characteristics of the respective third plurality of nucleic acid fragments. The values for the plurality of genomic characteristics in the respective third genomic data construct can include a respective second weighted mixture of (i) values for the plurality of genomic characteristics of the respective second plurality of nucleic acid fragments and (ii) values for the plurality of genomic characteristics of the respective third plurality of nucleic acid fragments. The respective second weighted mixture can be weighted more heavily towards the values for the plurality of the genomic characteristics of the respective third plurality of nucleic acid fragments than is the respective first weighted mixture.

The probability sampling can select a respective first portion of the respective second plurality of nucleic acid fragments that contribute to the values for the plurality of genotypic characteristics and a respective second portion of the respective third plurality of nucleic acid fragments that contribute to the values for the plurality of genotypic characteristics, and the magnitude of the respective first portion of nucleic acid fragments and the respective second portion of nucleic acid fragments is determined based on at least (i) the length of time between the first time point and the second time point and (ii) a temporal model for development of the respective state of the disease condition that the spike-in subject is afflicted with, in the set of states of the disease condition.

The respective second genomic data construct can be formed by mixing together a first amount of the second plurality of nucleic acid fragments from the second biological sample and a second amount of the cell-free nucleic acids from the spike-in biological sample, thereby forming a mixture of cell-free nucleic acids, sequencing nucleic acid fragments from the mixture of cell-free nucleic acids, and determining values for the plurality of genomic characteristics based on the sequencing. Accordingly, the method can include training a temporal classification algorithm against, for each respective training subject, at least the respective first genomic data construct, the respective second genomic data construct, the respective first time point and the respective second time point, or the derivation thereof, and the indication of the disease condition, at the respective first time and the respective second time point. In some embodiments, the temporal classification algorithm is further trained against the respective third genomic data construct, the respective third time point, or the derivation of the respective second time point and the respective second time point, and the indication of the state of the disease condition in the set of states of the disease condition, at the respective third time point, of the respective training subject. In some embodiments, the training data constructs include at least 3, 4, 5, 6, 7, 8, 9, 10, or more time points.

The method can further include evaluating a trained model using a titrated augmented data set, e.g., generated according to the sampling methods described above. The method can include obtaining a first classifier trained to discriminate a disease condition by evaluating a test genomic data construct (e.g., disease classifiers), where the test genomic data construct includes values for a plurality of genomic characteristics of a corresponding first plurality of nucleic acid fragments in a first corresponding biological sample obtained from the test subject. The method can then include obtaining an augmented assessment data set including a plurality of augmented genomic data constructs (e.g., augmented single time point data or augmented time-series data). Each respective augmented genomic data construct in the plurality of augmented genomic data constructs can include values for the plurality of genomic characteristics of a corresponding plurality of nucleic acid fragments representative of a corresponding biological sample obtained from a subject having a respective state of the disease condition in a plurality of states of the disease condition. The augmented assessment data set can include respective augmented genomic data constructs, in the plurality of augmented genotypic data constructs, that are representative of each respective state of the disease condition in the plurality of states of the disease condition, e.g., across a range spanning from no disease to advanced disease. The method can then include independently applying each respective augmented genomic data construct in the augmented assessment data set to the classifier to generate a disease state classification for each respective augmented genomic data construct, thereby generating a plurality of disease state classifications. The method can then include evaluating each respective disease state classification, in the plurality of disease state classifications, as a function of the respective state of the disease condition represented by the corresponding augmented genomic data construct, thereby assessing the performance of the classifier.

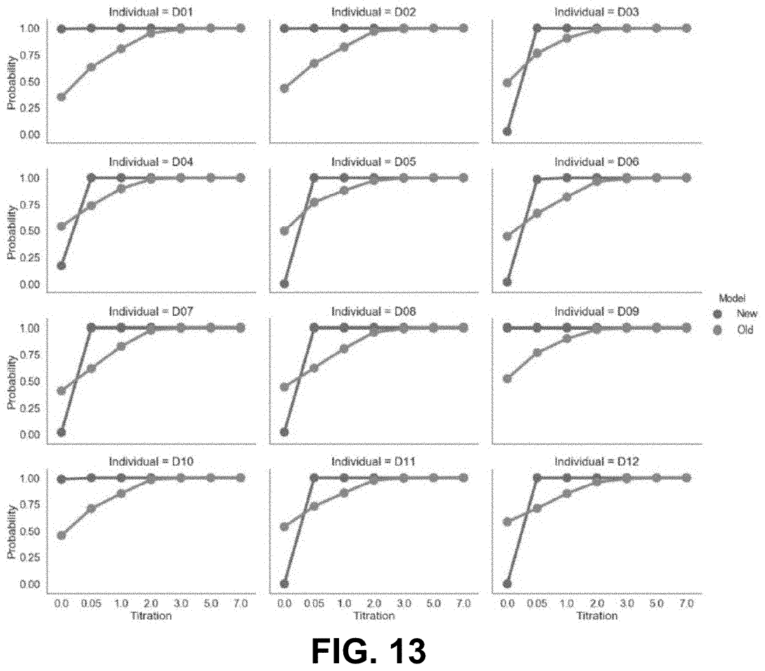

Generally, the disclosed methods may allow evaluation of the classifier across the range of disease states that may be represented within a population, to determine whether the classifier has been overfitted to the training data. For example, FIG. 13 illustrates the evaluation of two classifiers trained to detect cancer based on genomic characteristics of cell-free DNA in patient samples. Augmented time-series data constructs were prepared, according to the methods described herein, by diluting the biological cancer signal from samples of 12 cancer patients, forming a dilution series of tumor fractions down to 0%, e.g., completely lacking signal from any cancer cells. The dilution series data were then applied to the two classifiers, to produce probabilities (curves 802 and 804) that each data construct was generated from a sample of a cancer patient. As seen in FIG. 13, when the first classifier was used (corresponding to curve 802), several of the dilution series were classified as having a very high probability of being derived from a cancer patient, even when the augmented data construct contained no cancer signal at all (e.g., at titration=0), see individuals 1, 2, 9, and 10. This indicates that the models were overfitted to the training data, and would likely produce an unacceptable number of false positives. In contrast, when the second classifier was used (corresponding to curve 804), the cancer probabilities output by the model fell more gradually and more consistently for each augmented time-series, falling to or below 50% for all individuals, indicating that the model was less overfit than the first classifier.

In some embodiments, each state in the plurality of states of the cancer (e.g., in the plurality of augmented genotypic data constructs) includes a sub-range of cell-free DNA tumor fraction, in a range of cell-free DNA tumor fraction spanning at least from a baseline percentage of cell-free DNA tumor fraction that is at least 25% below the level of detection for the classifier to a ceiling percentage of cell-free DNA tumor fraction that is at least 25% above the level of detection for the classifier (624). In other embodiments, the sub-range of cell-free DNA tumor fraction falls within 5% of the level of detection for the classifier, or within 10%, 15%, 20%, 25%, 30%, 40%, or 50% of the level of detection for the classifier.

In some embodiments, each state in the plurality of states of the cardiovascular disease (e.g., in the plurality of augmented genotypic data constructs) includes a sub-range of cell-free DNA tumor fraction, in a range of cell-free DNA cardiovascular tissue fraction spanning at least from a baseline percentage of cell-free DNA cardiovascular tissue fraction that is at least 25% below the level of detection for the classifier to a ceiling percentage of cell-free DNA cardiovascular tissue fraction that is at least 25% above the level of detection for the classifier (628). In other embodiments, the sub-range of cell-free DNA tumor fraction falls within 5% of the level of detection for the classifier, or within 10%, 15%, 20%, 25%, 30%, 40%, or 50% of the level of detection for the classifier.

The classifier can include a logistic regression algorithm, a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

In some embodiments, a form of hard-negative mining is used to improve the performance of the classifier. For instance, the method includes obtaining a plurality of augmented false-positive genomic data constructs by identifying a subset of genomic data constructs from the second plurality of genomic data constructs that are discriminated by a precursor to the classifier with a performance that fails a performance threshold, and using the subset of genomic data constructs to generate the plurality of augmented false-positive genotypic data constructs. Each respective augmented false-positive genomic data construct can correspond to at least a respective genomic data construct from the sub-set of genomic data constructs, and each respective genomic data construct in the plurality of augmented false-positive genomic data constructs can include an augmented value that is derived from probability sampling of nucleic acid fragments contributing to the value of the respective genomic characteristic in at least the respective genomic data construct from the sub-set of genomic data constructs. In these embodiments, the classifier can be further trained against the plurality of augmented false-positive genomic data constructs and an indication of the state of the disease condition.

Many different models can evaluate biological features in order to classify one or more disease statuses (e.g., a cancer status, coronary disease status, etc.) of a subject. For instance, U.S. Patent Application Publication No. 2019/0287652 describes models that evaluate the methylation status across a plurality of genomic loci, e.g., using cfDNA samples, in order to classify a cancer status of a subject. Similarly, U.S. Patent Application Publication No. 2019/0287649 describes models that evaluate the relative copy number across a plurality of genomic loci, e.g., using cfDNA samples, in order to classify a cancer status of a subject. Likewise, various models have been developed that evaluate the presence of variant alleles (e.g., single nucleotide variants, indels, deletions, transversions, translocations, etc.) in order to classify a cancer status of a subject. Other suitable models are disclosed in U.S. patent application Ser. No. 16/428,575 entitled “Convolutional Neural Network Systems and Methods for Data Classification,” filed May 31, 2019. Generally, any model developed for the classification of a disease status of a subject may be trained using the augmented data sets described herein and used in conjunction with the systems and methods described herein, e.g., for determining the disease state of a test subject.

A classifier can be for detecting the presence of a disease state in a subject, e.g., detecting cancer or coronary disease in a subject. The systems and methods provided herein can be suited for improving upon the sensitivity and specificity of existing disease models, because they can be trained using additional augmented data that provide many examples of weak disease signals near the limit of detection for models trained on patient data. Because of the expense associated with collecting training data, and the fact that patient data is not often collected at early stages of a disease, training data sets may not include many data constructs with disease signals around the limit of detection for the model. Rather, training sets may have many examples of strong disease signals from training subjects with an advanced disease state and many examples of no disease signals from training subjects that do not have the disease. However, because it is difficult to positively diagnose the early stages of a disease, training data sets may include few moderate to weak disease signals, which are important for improving the sensitivity and specificity of the classifier.

Generally, many different classification algorithms can be used in the systems and methods described herein. For instance, the model can include a neural network algorithm, a support vector machine algorithm, a Naive Bayes algorithm, a nearest neighbor algorithm, a boosted trees algorithm, a random forest algorithm, a decision tree algorithm, a regression algorithm, a multinomial logistic regression algorithm, a linear model, or a linear regression algorithm. Use of augmented data constructs can improve the performance of regression-based models more than it improves the performance of the classifier than for deeper learning-based models, e.g., neural networks. The regression algorithm can be logistic regression with lasso, L2 or elastic net regularization. In some embodiments, the logistic regression further includes personal characteristics, for example, one or more of gender, age, family medical history, personal medical history, ethnicity, smoking status, alcohol consumption status, anthropomorphic data, etc.

The disease state model can include learned weights for the features that are adjusted during training. The term “weights” can be used generically here to represent the learned quantity associated with any given feature of a model, regardless of which particular machine learning technique is used. In some embodiments, a cancer indicator score is determined by inputting values for features derived from one or more DNA sequences (or DNA sequence reads thereof) into a machine learning or deep learning model. In some embodiments, e.g., when the disease class evaluation model is a neural network (e.g., a conventional or convolutional neural network), the output of a disease classifier is a classification, e.g., either cancer positive or cancer negative. However, in order to provide a continuous or semi-continuous value for the output of the model, rather than a classification, a hidden layer of a neural network, e.g., the hidden layer just prior to the output layer, can be used as the output of the classification model.

Accordingly, the model can include (i) an input layer for receiving values for the plurality of genomic characteristics, where the plurality of genomic characteristics includes a first number of dimensions, and (ii) an embedding layer that includes a set of weights, where the embedding layer directly or indirectly receives output of the input layer, and where an output of the embedding layer is a model score set having a second number of dimensions that is less than the first number of dimensions, and (iii) an output layer that directly or indirectly receives the model score set from the embedding layer. In such embodiments, the first model score set is the model score set of the embedding layer upon inputting the first genomic data construct into the input layer, and the second model score set is the model score set of the embedding layer upon inputting the second genomic data construct into the input layer. In other words, the model score set can be the output of a set of neurons associated with a hidden layer in a neural network termed the embedding layer. Each such neuron in the embedding layer can be associated with a weight and an activation function and the model score set consists of the output of each such activation function. The activation function of a neuron in the embedding layer can be a rectified linear unit (ReLU), a tan h function, or a sigmoid activation function. In some such embodiments, the neurons of the embedding layer can be fully connected to each of the inputs of the input layer. Each neuron of the output layer can be fully connected to each neuron of the embedding layer. Each neuron of the output layer can be associated with a Softmax activation function. In some embodiments, one or more of the embedding layers and the output layers are not fully connected.

III.D. Deployment of Cancer Classifier

During use of the cancer classifier, the analytics system can obtain a test sample from a subject of unknown cancer type. The analytics system may process the test sample comprised of DNA molecules with any combination of the processes 100, 200, and 220 to achieve a set of anomalous fragments. The analytics system can determine a test feature vector for use by the cancer classifier according to similar principles discussed in the process 500. The analytics system can calculate an anomaly score for each CpG site in a plurality of CpG sites in use by the cancer classifier. For example, the cancer classifier receives as input feature vectors inclusive of anomaly scores for 1,000 selected CpG sites. The analytics system can thus determine a test feature vector inclusive of anomaly scores for the 1,000 selected CpG sites based on the set of anomalous fragments. The analytics system can calculate the anomaly scores in a same manner as the training samples. In some embodiments, the analytics system defines the anomaly score as a binary score based on whether there is a hypermethylated or hypomethylated fragment in the set of anomalous fragments that encompasses the CpG site.

The analytics system can then input the test feature vector into the cancer classifier. The function of the cancer classifier can then generate a cancer prediction based on the classification parameters trained in the process 600 and the test feature vector. In the first manner, the cancer prediction can be binary and selected from a group consisting of “cancer” or non-cancer;” in the second manner, the cancer prediction is selected from a group of many cancer types and “non-cancer.” In additional embodiments, the cancer prediction has predictions values for each of the many cancer types. Moreover, the analytics system may determine that the test sample is most likely to be of one of the cancer types. Following the example above with the cancer prediction for a test sample as 65% likelihood of breast cancer, 25% likelihood of lung cancer, and 10% likelihood of non-cancer, the analytics system may determine that the test sample is most likely to have breast cancer. In another example, where the cancer prediction is binary as 60% likelihood of non-cancer and 40% likelihood of cancer, the analytics system determines that the test sample is most likely not to have cancer. In additional embodiments, the cancer prediction with the highest likelihood may still be compared against a threshold (e.g., 40%, 50%, 60%, 70%) in order to call the test subject as having that cancer type. If the cancer prediction with the highest likelihood does not surpass that threshold, the analytics system may return an inconclusive result.

In additional embodiments, the analytics system chains a cancer classifier trained in step 560 of the process 600 with another cancer classifier trained in step 570 or the process 500. The analytics system can input the test feature vector into the cancer classifier trained as a binary classifier in step 560 of the process 600. The analytics system can receive an output of a cancer prediction. The cancer prediction may be binary as to whether the test subject likely has or likely does not have cancer. In other implementations, the cancer prediction includes prediction values that describe likelihood of cancer and likelihood of non-cancer. For example, the cancer prediction has a cancer prediction value of 85% and the non-cancer prediction value of 15%. The analytics system may determine the test subject to likely have cancer. Once the analytics system determines a test subject is likely to have cancer, the analytics system may input the test feature vector into a multiclass cancer classifier trained to distinguish between different cancer types. The multiclass cancer classifier can receive the test feature vector and returns a cancer prediction of a cancer type of the plurality of cancer types. For example, the multiclass cancer classifier provides a cancer prediction specifying that the test subject is most likely to have ovarian cancer. In another implementation, the multiclass cancer classifier provides a prediction value for each cancer type of the plurality of cancer types. For example, a cancer prediction may include a breast cancer type prediction value of 40%, a colorectal cancer type prediction value of 15%, and a liver cancer prediction value of 45%.

According to generalized embodiment of binary cancer classification, the analytics system can determine a cancer score for a test sample based on the test sample's sequencing data (e.g., methylation sequencing data, SNP sequencing data, other DNA sequencing data, RNA sequencing data, etc.). The analytics system can compare the cancer score for the test sample against a binary threshold cutoff for predicting whether the test sample likely has cancer. The binary threshold cutoff can be tuned using TOO thresholding based on one or more TOO subtype classes. The analytics system may further generate a feature vector for the test sample for use in the multiclass cancer classifier to determine a cancer prediction indicating one or more likely cancer types.

The classifier may be used to determine the disease state of a test subject, e.g., a subject whose disease status is unknown. The method can include obtaining a test genomic data construct (e.g., single time point test data), in electronic form, that includes a value for each genomic characteristic in the plurality of genomic characteristics of a corresponding plurality of nucleic acid fragments in a biological sample obtained from a test subject. The method can then include applying the test genomic data construct to the test classifier to thereby determine the state of the disease condition in the test subject. The test subject may not be previously diagnosed with the disease condition.

The classifier can be a temporal classifier that uses at least (i) a first test genomic data construct generated from a first biological sample acquired from a test subject at a first point in time, and (ii) a second test genomic data construct generated from a second biological sample acquired from a test subject at a second point in time.

The trained classifier can be used to determine the disease state of a test subject, e.g., a subject whose disease status is unknown. In this case, the method can include obtaining a test time-series data set, in electronic form, for a test subject, where the test time-series data set includes, for each respective time point in a plurality of time points, a corresponding test genotypic data construct including values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points. The method can then include applying the test genotypic data construct to the test classifier to thereby determine the state of the disease condition in the test subject. The test subject may not be previously diagnosed with the disease condition.

### IV. Applications

In some embodiments, the methods, analytic systems and/or classifier of the present invention can be used to detect the presence of cancer, monitor cancer progression or recurrence, monitor therapeutic response or effectiveness, determine a presence or monitor minimum residual disease (MRD), or any combination thereof. For example, as described herein, a classifier can be used to generate a probability score (e.g., from 0 to 100) describing a likelihood that a test feature vector is from a subject with cancer. In some embodiments, the probability score is compared to a threshold probability to determine whether or not the subject has cancer. In other embodiments, the likelihood or probability score can be assessed at multiple different time points (e.g., before or after treatment) to monitor disease progression or to monitor treatment effectiveness (e.g., therapeutic efficacy). In still other embodiments, the likelihood or probability score can be used to make or influence a clinical decision (e.g., diagnosis of cancer, treatment selection, assessment of treatment effectiveness, etc.). For example, in one embodiment, if the probability score exceeds a threshold, a physician can prescribe an appropriate treatment.

IV.A. Early Detection of Cancer

In some embodiments, the methods and/or classifier of the present invention are used to detect the presence or absence of cancer in a subject suspected of having cancer. For example, a classifier (e.g., as described above in Section III and exampled in Section V) can be used to determine a cancer prediction describing a likelihood that a test feature vector is from a subject that has cancer.

In one embodiment, a cancer prediction is a likelihood (e.g., scored between 0 and 100) for whether the test sample has cancer (i.e. binary classification). Thus, the analytics system may determine a threshold for determining whether a test subject has cancer. For example, a cancer prediction of greater than or equal to 60 can indicate that the subject has cancer. In still other embodiments, a cancer prediction greater than or equal to 65, greater than or equal to 70, greater than or equal to 75, greater than or equal to 80, greater than or equal to 85, greater than or equal to 90, or greater than or equal to 95 indicates that the subject has cancer. In other embodiments, the cancer prediction can indicate the severity of disease. For example, a cancer prediction of 80 may indicate a more severe form, or later stage, of cancer compared to a cancer prediction below 80 (e.g., a probability score of 70). Similarly, an increase in the cancer prediction over time (e.g., determined by classifying test feature vectors from multiple samples from the same subject taken at two or more time points) can indicate disease progression or a decrease in the cancer prediction over time can indicate successful treatment.

In another embodiment, a cancer prediction comprises many prediction values, wherein each of a plurality of cancer types being classified (i.e. multiclass classification) for has a prediction value (e.g., scored between 0 and 100). The prediction values may correspond to a likelihood that a given training sample (and during inference, training sample) has each of the cancer types. The analytics system may identify the cancer type that has the highest prediction value and indicate that the test subject likely has that cancer type. In other embodiments, the analytics system further compares the highest prediction value to a threshold value (e.g., 50, 55, 60, 65, 70, 75, 80, 85, etc.) to determine that the test subject likely has that cancer type. In other embodiments, a prediction value can also indicate the severity of disease. For example, a prediction value greater than 80 may indicate a more severe form, or later stage, of cancer compared to a prediction value of 60. Similarly, an increase in the prediction value over time (e.g., determined by classifying test feature vectors from multiple samples from the same subject taken at two or more time points) can indicate disease progression or a decrease in the prediction value over time can indicate successful treatment.

According to aspects of the invention, the methods and systems of the present invention can be trained to detect or classify multiple cancer indications. For example, the methods, systems and classifiers of the present invention can be used to detect the presence of one or more, two or more, three or more, five or more, ten or more, fifteen or more, or twenty or more different types of cancer.

Examples of cancers that can be detected using the methods, systems and classifiers of the present invention include carcinoma, lymphoma, blastoma, sarcoma, and leukemia or lymphoid malignancies. More particular examples of such cancers include, but are not limited to, squamous cell cancer (e.g., epithelial squamous cell cancer), skin carcinoma, melanoma, lung cancer, including small-cell lung cancer, non-small cell lung cancer (“NSCLC”), adenocarcinoma of the lung and squamous carcinoma of the lung, cancer of the peritoneum, gastric or stomach cancer including gastrointestinal cancer, pancreatic cancer (e.g., pancreatic ductal adenocarcinoma), cervical cancer, ovarian cancer (e.g., high grade serous ovarian carcinoma), liver cancer (e.g., hepatocellular carcinoma (HCC)), hepatoma, hepatic carcinoma, bladder cancer (e.g., urothelial bladder cancer), testicular (germ cell tumor) cancer, breast cancer (e.g., HER2 positive, HER2 negative, and triple negative breast cancer), brain cancer (e.g., astrocytoma, glioma (e.g., glioblastoma)), colon cancer, rectal cancer, colorectal cancer, endometrial or uterine carcinoma, salivary gland carcinoma, kidney or renal cancer (e.g., renal cell carcinoma, nephroblastoma or Wilms' tumor), prostate cancer, vulval cancer, thyroid cancer, anal carcinoma, penile carcinoma, head and neck cancer, esophageal carcinoma, and nasopharyngeal carcinoma (NPC). Additional examples of cancers include, without limitation, retinoblastoma, thecoma, arrhenoblastoma, hematological malignancies, including but not limited to non-Hodgkin's lymphoma (NHL), multiple myeloma and acute hematological malignancies, endometriosis, fibrosarcoma, choriocarcinoma, laryngeal carcinomas, Kaposi's sarcoma, Schwannoma, oligodendroglioma, neuroblastomas, rhabdomyosarcoma, osteogenic sarcoma, leiomyosarcoma, and urinary tract carcinomas.

In some embodiments, the cancer is one or more of anorectal cancer, bladder cancer, breast cancer, cervical cancer, colorectal cancer, esophageal cancer, gastric cancer, head & neck cancer, hepatobiliary cancer, leukemia, lung cancer, lymphoma, melanoma, multiple myeloma, ovarian cancer, pancreatic cancer, prostate cancer, renal cancer, thyroid cancer, uterine cancer, or any combination thereof.

In some embodiments, the one or more cancer can be a “high-signal” cancer (defined as cancers with greater than 50% 5-year cancer-specific mortality), such as anorectal, colorectal, esophageal, head & neck, hepatobiliary, lung, ovarian, and pancreatic cancers, as well as lymphoma and multiple myeloma. High-signal cancers tend to be more aggressive and typically have an above-average cell-free nucleic acid concentration in test samples obtained from a patient.

IV.B. Cancer and Treatment Monitoring

In some embodiments, the cancer prediction can be assessed at multiple different time points (e.g., or before or after treatment) to monitor disease progression or to monitor treatment effectiveness (e.g., therapeutic efficacy). For example, the present invention include methods that involve obtaining a first sample (e.g., a first plasma cfDNA sample) from a cancer patient at a first time point, determining a first cancer prediction therefrom (as described herein), obtaining a second test sample (e.g., a second plasma cfDNA sample) from the cancer patient at a second time point, and determining a second cancer prediction therefrom (as described herein).

In certain embodiments, the first time point is before a cancer treatment (e.g., before a resection surgery or a therapeutic intervention), and the second time point is after a cancer treatment (e.g., after a resection surgery or therapeutic intervention), and the classifier is utilized to monitor the effectiveness of the treatment. For example, if the second cancer prediction decreases compared to the first cancer prediction, then the treatment is considered to have been successful. However, if the second cancer prediction increases compared to the first cancer prediction, then the treatment is considered to have not been successful. In other embodiments, both the first and second time points are before a cancer treatment (e.g., before a resection surgery or a therapeutic intervention). In still other embodiments, both the first and the second time points are after a cancer treatment (e.g., after a resection surgery or a therapeutic intervention). In still other embodiments, cfDNA samples may be obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy.

Those of skill in the art will readily appreciate that test samples can be obtained from a cancer patient over any desired set of time points and analyzed in accordance with the methods of the invention to monitor a cancer state in the patient. In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 50 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, test samples can be obtained from the patient at least once every 5 months, at least once every 6 months, at least once a year, at least once every 2 years, at least once every 3 years, at least once every 4 years, or at least once every 5 years.

IV.C. Treatment

In still another embodiment, the cancer prediction can be used to make or influence a clinical decision (e.g., diagnosis of cancer, treatment selection, assessment of treatment effectiveness, etc.). For example, in one embodiment, if the cancer prediction (e.g., for cancer or for a particular cancer type) exceeds a threshold, a physician can prescribe an appropriate treatment (e.g., a resection surgery, radiation therapy, chemotherapy, and/or immunotherapy).

A classifier (as described herein) can be used to determine a cancer prediction that a sample feature vector is from a subject that has cancer. In one embodiment, an appropriate treatment (e.g., resection surgery or therapeutic) is prescribed when the cancer prediction exceeds a threshold. For example, in one embodiment, if the cancer prediction is greater than or equal to 60 one or more appropriate treatments are prescribed. In another embodiment, if the cancer prediction is greater than or equal to 65, greater than or equal to 70, greater than or equal to 75, greater than or equal to 80, greater than or equal to 85, greater than or equal to 90, or greater than or equal to 95, one or more appropriate treatments are prescribed. In other embodiments, the cancer prediction can indicate the severity of disease. An appropriate treatment matching the severity of the disease may then be prescribed.

In some embodiments, the treatment is one or more cancer therapeutic agents selected from the group consisting of a chemotherapy agent, a targeted cancer therapy agent, a differentiating therapy agent, a hormone therapy agent, and an immunotherapy agent. For example, the treatment can be one or more chemotherapy agents selected from the group consisting of alkylating agents, antimetabolites, anthracyclines, anti-tumor antibiotics, cytoskeletal disruptors (taxans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group consisting of signal transduction inhibitors (e.g. tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group consisting of anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). It is within the capabilities of a skilled physician or oncologist to select an appropriate cancer therapeutic agent based on characteristics such as the type of tumor, cancer stage, previous exposure to cancer treatment or therapeutic agent, and other characteristics of the cancer.

### V. Example Results of Cancer Classifier

V.A. Sample Collection and Processing

Study design and samples: CCGA (NCT02889978) is a prospective, multi-center, case-control, observational study with longitudinal follow-up. De-identified biospecimens were collected from approximately 15,000 participants from 142 sites. Samples were divided into training (1,785) and test (1,015) sets; samples were selected to ensure a prespecified distribution of cancer types and non-cancers across sites in each cohort, and cancer and non-cancer samples were frequency age-matched by gender.

Whole-genome bisulfite sequencing: cfDNA was isolated from plasma, and whole-genome bisulfite sequencing (WGBS; 30× depth) was employed for analysis of cfDNA. cfDNA was extracted from two tubes of plasma (up to a combined volume of 10 ml) per patient using a modified QIAamp Circulating Nucleic Acid kit (Qiagen; Germantown, Md.). Up to 75 ng of plasma cfDNA was subjected to bisulfite conversion using the EZ-96 DNA Methylation Kit (Zymo Research, D5003). Converted cfDNA was used to prepare dual indexed sequencing libraries using Accel-NGS Methyl-Seq DNA library preparation kits (Swift BioSciences; Ann Arbor, Mich.) and constructed libraries were quantified using KAPA Library Quantification Kit for Illumina Platforms (Kapa Biosystems; Wilmington, Mass.). Four libraries along with 10% PhiX v3 library (Illumina, FC-110-3001) were pooled and clustered on an Illumina NovaSeq 7000 S2 flow cell followed by 150-bp paired-end sequencing (30×).

For each sample, the WGBS fragment set was reduced to a small subset of fragments having an anomalous methylation pattern. Additionally, hyper or hypomethylated cfDNA fragments were selected. cfDNA fragments selected for having an anomalous methylation pattern and being hyper or hypermethylated, i.e., UFXM. Fragments occurring at high frequency in individuals without cancer, or that have unstable methylation, are unlikely to produce highly discriminatory features for classification of cancer status. We therefore produced a statistical model and a data structure of typical fragments using an independent reference set of 108 non-smoking participants without cancer (age: 58±14 years, 79 [73%] women) (i.e., a reference genome) from the CCGA study. These samples were used to train a Markov-chain model (order 3) estimating the likelihood of a given sequence of CpG methylation statuses within a fragment as described above in Section II.B. This model was demonstrated to be calibrated within the normal fragment range (p-value>0.001) and was used to reject fragments with a p-value from the Markov model as >=0.001 as insufficiently unusual.

As described above, further data reduction step selected only fragments with at least 5 CpGs covered, and average methylation either >0.9 (hyper methylated) or <0.1 (hypomethylated). This procedure resulted in a median (range) of 2,800 (1,500-12,000) UFXM fragments for participants without cancer in training, and a median (range) of 3,000 (1,200-220,000) UFXM fragments for participants with cancer in training. As this data reduction procedure only used reference set data, this stage was only required to be applied to each sample once.

V.B. Cancer Classification

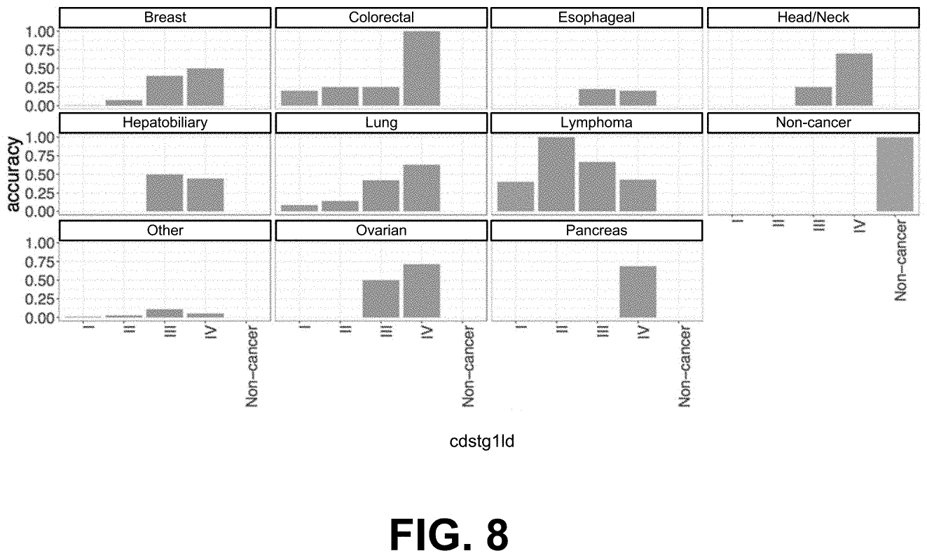

FIGS. 8-11 illustrate graphs showing cancer prediction accuracy of trained cancer classifiers, according to example implementations. The cancer classifiers used to produce results shown in FIGS. 8-11 are trained according to example implementations of the processes described in Section III, or some combination thereof.

The analytics system selects CpG sites to be considered in the cancer classifier. The information gain is computed for training samples with a given cancer type compared to all other samples. For example, two random variables ‘anomalous fragment’ (‘AF’) and ‘cancer type’ (CT′) are used. CT is a random variable indicating whether the cancer is of a particular type. The analytics system computes the mutual information with respect to CT given AF. That is, how many bits of information about the cancer type are gained if it is known whether there is an anomalous fragment overlapping a particular CpG site. For a given cancer type, the analytics system uses this information to rank CpG sites based on how cancer specific they are. This procedure is repeated for all cancer types under consideration. The ranked CpG sites for each cancer type are greedily added (e.g., to achieve approximately 3,000 CpG sites) for use in the cancer classifier.

For featurization of samples, the analytics system identifies fragments in each sample with anomalous methylation patterns and furthermore UFXM fragments. For one sample, the analytics system calculates an anomaly score for each selected CpG site for consideration (˜3,000). The analytics system defines the anomaly score with a binary scoring based on whether the sample has a UFXM fragment that encompasses the CpG site.

FIG. 8 illustrates graphs showing cancer prediction accuracy of a multiclass cancer classifier for various cancer types, according to an example implementation. In this illustrative example, the multiclass cancer classifier is trained to distinguish feature vectors according to 11 cancer types: breast cancer type, colorectal cancer type, esophageal cancer type, head/neck cancer type, hepatobiliary cancer type, lung cancer type, lymphoma cancer type, ovarian cancer type, pancreas cancer type, non-cancer type, and other cancer type. The samples used in this example were from subjects known to have each of the cancer types. For example, a cohort of breast cancer type samples were used to validate the cancer classifier's accuracy in calling the breast cancer type. Moreover, the samples used are from subjects in varying stages of cancer.

For the breast cancer cohort, the colorectal cancer cohort, and the lung cancer cohort, the cancer classifier was gradually more accurate in accurately predicting the cancer type in subsequent stages of cancer. For the head/neck cohort, ovarian cohort, and pancreas cohort, the cancer classifier had accuracy increases in the latter stage, i.e., Stage III and/or Stage IV. For the esophageal cohort and the hepatobiliary cohort, the cancer classifier also had latter stage accuracy, i.e., Stage III and Stage IV. With the non-cancer cohort, the cancer classifier was perfectly accurate in predicting the non-cancer samples to not likely have cancer. Last but not least, the lymphoma cohort had success throughout varying stages with a peak success in accurately predicting samples in Stage II of cancer.

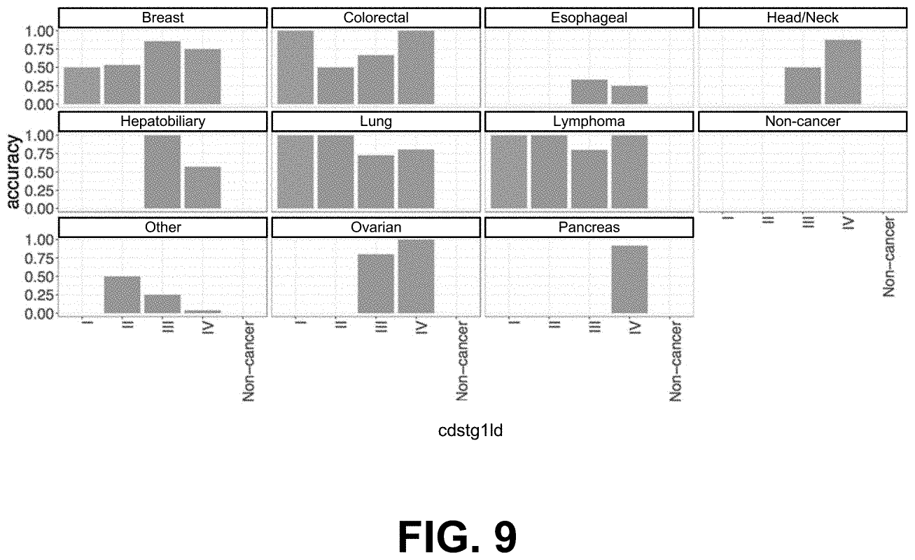

FIG. 9 illustrates graphs showing cancer prediction accuracy of a multiclass cancer classifier for various cancer types after first using a binary cancer classifier, according to an example implementation. In this example, the analytics system first inputs the samples from many cancer type cohorts into the binary cancer classifier to determine whether or not the samples likely have or do not have cancer. Then the analytics system inputs samples that are determined to likely have cancer into the multiclass cancer classifier to predict a cancer type for those samples. The cancer types in consideration include: breast cancer type, colorectal cancer type, esophageal cancer type, head/neck cancer type, hepatobiliary cancer type, lung cancer type, lymphoma cancer type, ovarian cancer type, pancreas cancer type, and other cancer type.

In comparison to the example in FIG. 8, the analytics system showed an increase in accuracy when first using the binary cancer classifier then the multiclass cancer classifier. Among the breast cancer cohort, the colorectal cancer cohort, the lung cancer cohort, and the lymphoma cancer cohort, the analytics system had overall increases in accuracy. In particular, the analytics system had stark increases in prediction accuracy for each of those cancer types in early stages of cancer, i.e., Stage I, Stage II, and even Stage III.

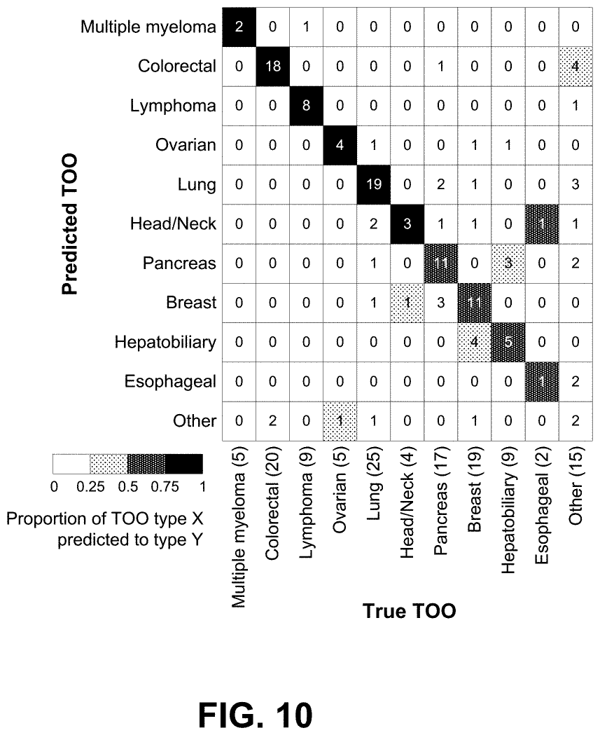

FIG. 10 illustrates a confusion matrix demonstrating performance of a trained cancer classifier, according to an example implementation. In one example of training according to the process 500, a multiclass kernel logistic regression (KLR) classifier with ridge regression penalty was trained on the derived feature vectors with a penalty on the weights, and a fixed penalty on the bias term for each cancer type. The ridge regression penalty was optimized on a portion of the training data not used in selecting high-relevance locations (using log-loss), and, once the optimum parameter was found, the logistic classifier was retrained on the whole set of local training folds. The selected high-relevance sites and classifier weights were then applied to new data. Within the CCGA training set, one fold was repeatedly held out, relevant sites on 8 of the 9 folds were selected, the hyper-parameters for the KLR classifier were optimized on the 9th set, and the KLR was retrained on 9 of 10 folds and applied to the held-out fold. This was repeated 10 times to estimate TOO within the CCGA training set. For the CCGA test set, relevant sites were selected on 9/10 folds of CCGA train, hyper-parameters were optimized on the 10th fold, and the KLR classifier was retrained on all CCGA training data and the selected sites and the KLR classifier were applied to the test set. The cancer types considered include: multiple myeloma cancer type, colorectal cancer type, lymphoma cancer type, ovarian cancer type, lung head/neck cancer type, pancreas cancer type, breast cancer type, hepatobiliary cancer type, esophageal cancer type, and other cancer type. Other cancer type included cancers with less than 5 samples collected within CCGA, such as anorectal, bladder, cancer of unknown primary TOO, cervical, gastric, leukemia, melanoma, prostate, renal thyroid, uterine, and other additional cancers.

The confusion matrix shows agreement between cancer types having samples with known cancer TOO (along x-axis) and predicted cancer TOO (along y-axis). To validate performance of the trained KLR classifier, a cohort of samples (indicated in parentheses along the y-axis for each cancer type) for each cancer type was classified with the KLR classifier. The x-axis indicates how many samples from each cohort was classified under each cancer type. For example, with the lung cancer cohort having 25 samples with known lung cancer, the KLR classifier predicted one sample to have ovarian cancer, nineteen samples to have lung cancer, two samples to have head/neck cancer, one sample to have pancreas cancer, one sample to have breast cancer, and one sample to be labeled as other cancer type. Notably, for all cancer types except other cancer type, the KLR classifier accurately predicted more than half of each cohort with particularly high accuracy for the cancer types of multiple myeloma (2/2 or 100%), colorectal (18/20 or 90%), lymphoma (8/9 or 88.8%), ovarian (4/5 or 80%), lung (19/25 or 76%), and head/neck (3/4 or 75%). These results demonstrate the predictive accuracy of the KLR classifier.

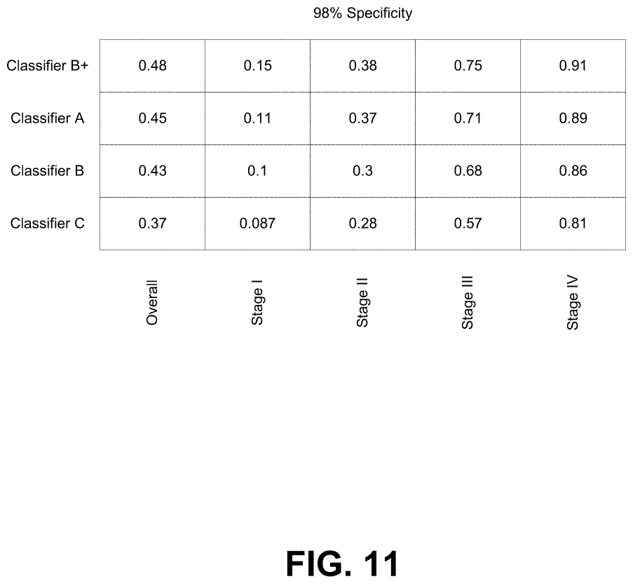

FIG. 11 illustrates a table comparing performance of a cancer classifier trained with synthetic training samples, according to some example implementations. Classifier A is trained with feature vectors generated according to FIG. 6B. Classifier B is trained with feature vectors generated according to methodology described in U.S. application Ser. No. 16/579,805 entitled “Mixture Model for Targeted Sequencing.” Classifier B+ refers to implementation of featurization as in Classifier B with the added synthetic training samples. The variously trained classifiers were evaluated with holdout sets at a 98% specificity threshold. Classifier B+ overall performed the best with a sensitivity of 0.48. Across various stages of cancer, the Classifier B+ also performed better than the other classifiers with sensitivity of 0.15 in Stage I samples, sensitivity of 0.38 in Stage II samples, sensitivity of 0.75 in Stage III samples, and sensitivity of 0.91 in Stage IV samples.

The data used in the analyses presented in the examples below was collected as part of the CCGA clinical study. CCGA [NCT02889978] is a prospective, multi-center, observational cfDNA-based early cancer detection study that has enrolled over 15,000 demographically-balanced participants at over 140 sites. Blood samples were collected from newly diagnosed therapy-naive cancer (C, case) and participants without a diagnosis of cancer (noncancer [NC], control) as defined at enrollment.

Three sequencing assays were performed on the blood drawn from each participant: 1) paired cfDNA and white blood cell (WBC)-targeted sequencing (60,000×, 507 gene panel) for single nucleotide variants/indels (the ART sequencing assay); a joint caller removed WBC-derived somatic variants and residual technical noise; 2) paired cfDNA and WBC whole-genome sequencing (WGS; 35×) for copy number variation; a novel machine learning algorithm generated cancer-related signal scores; joint analysis identified shared events; and 3) cfDNA whole-genome bisulfite sequencing (WGBS; 34×) for methylation; normalized scores were generated using abnormally methylated fragments. In addition, tissue samples were obtained from participants with cancer only, such that 4) whole-genome sequencing (WGS; 30×) was performed on paired tumor and WBC gDNA for identification of tumor variants for comparison.

### Example 1—in Silico Spiking of Cancer Signals into Data from Non-Cancerous Subjects

An in silico data spiking experiment was designed to test the effect of spiking the same amount of various cancer signals into different biological backgrounds. In the experiment, increasing percentages of binned counts for nucleic acid fragment sequences mapped to respective genomic regions in a plurality of genomic regions from subjects known to have various types of cancer were serially spiked into corresponding binned counts determined for nucleic acid fragment sequences mapped to the sample plurality of genomic regions for subjects with very low tumor fractions. Advantageously, there is no requirement that the genetic loci, or the alleles of these genetic loci that harbor the cancer signal, be known.

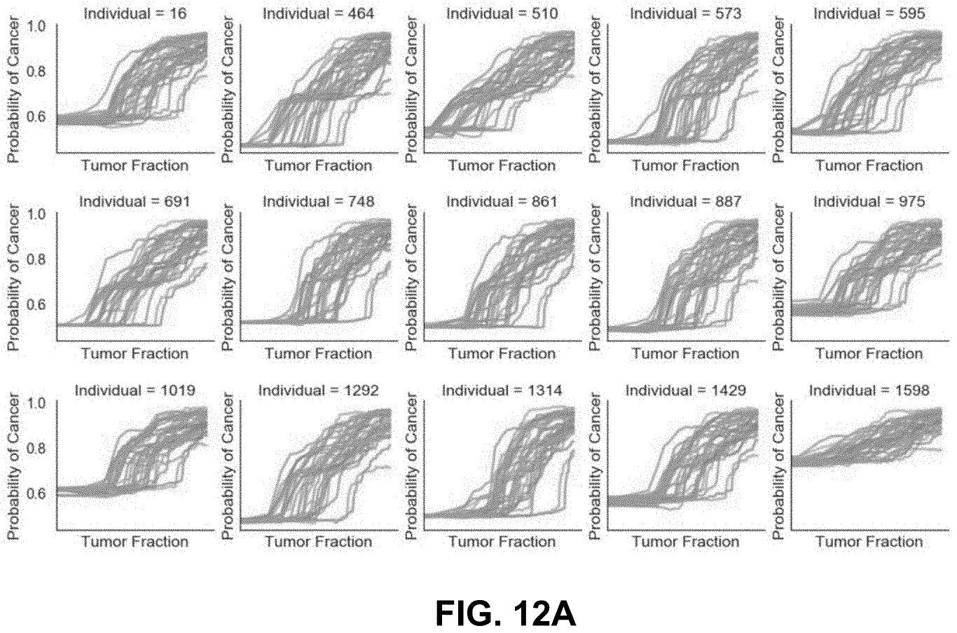

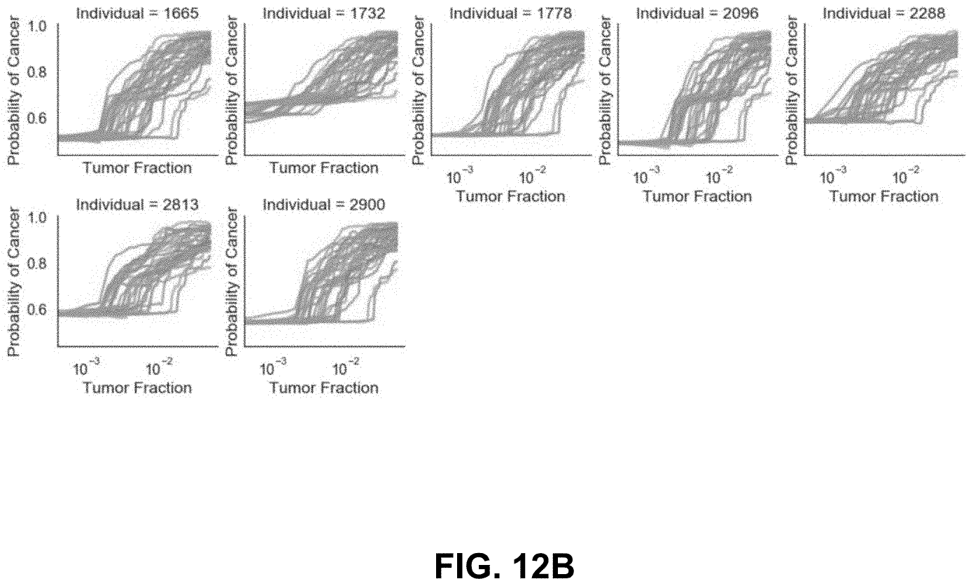

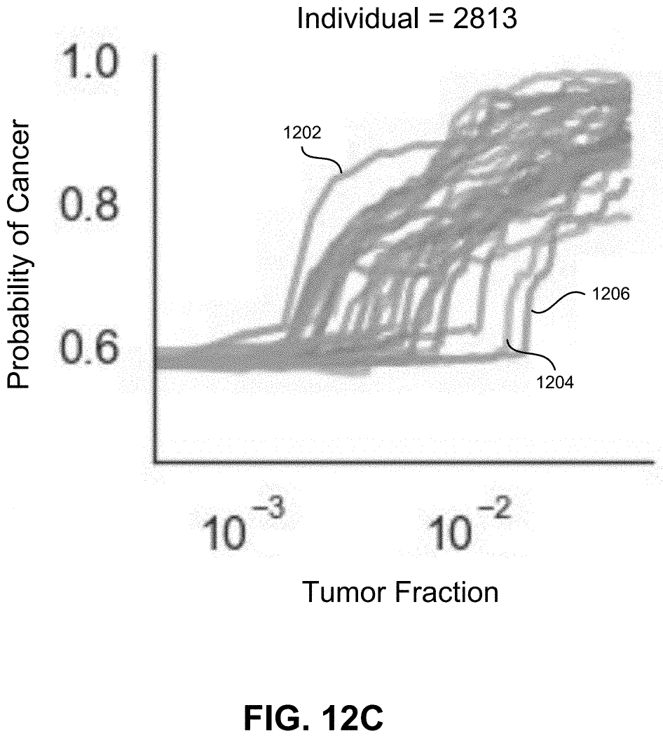

In this way, a time-series development of cancer, in silico, was created. Development of the cancer signal, as reported by a probability of cancer derived from a cancer classifier trained against relative bin values (Y-axis in each of the plots in FIGS. 12A-12C), was evaluated for each spiked data sample. The classifier used in this experiment is described in U.S. Patent Application Publication No. 2019/0287649, which is hereby incorporated by reference.

Twenty-two CCGA low-tumor-fraction subjects with undetectable levels of cell-free tumor fraction were matched with twenty-two high-tumor-fraction subjects who were known to have different types of cancer, who each had a cell-free DNA tumor fraction of at least 10%, and for whom the cancer classifier provide at least a 90% probability of having cancer, were selected from the CCGA study data. Increasing amounts of bin counts from each of the high-tumor-fraction subjects were added to the corresponding bin counts of low-tumor-fraction subjects, forming four hundred and eighty-four sets of cancer series data having increasing bin counts, as plotted on the X-axis of each of the graphs shown in FIGS. 12A-12C. Such bin counts represent the number of sequences observed in a sample that map to a particular bin, where each bin represents a unique portion of a reference human genome. As such, such bin counts are considered as a form of copy number variation 133 (FIG. 1B). To illustrate, in FIGS. 12A-12CC, individual 2813 is one of the twenty-two CCGA low-tumor-fraction subjects. For this individual, there are twenty-two lines in the illustrated graph. Each respective line in the graph represents the progressive spiking (X-axis) of the corresponding allelic counts of a respective high-tumor-fraction subject in the set of twenty-two high-tumor-fraction subjects. For instance, line 702 represents the progressive spiking of low-tumor-fraction subject 2813 with the bin counts of a first high-tumor-fraction subject, line 704 represents the progressive spiking of low-tumor-fraction subject 2813 with the bin counts of a second high-tumor-fraction subject cancer, line 706 represents the progressive spiking of low-tumor-fraction subject 2813 with the bin counts of a third high-tumor-fraction subject, and so forth. Each of the four hundred and eighty-four sets of cancer series data includes a plurality of two-dimensional points (x, y), where x=target_TF, and y is the probability of having cancer returned by a trained classifier upon inputting to the trained classifier the bin count data for the respective point, and where the bin counts data includes a respective bin count (counts_newᵢ) for each bin i in a plurality of bins computed as:

counts_newᵢ=(target_TF/actual_TF)*counts_highTFᵢ+(1−target_TF/actual_TF)*counts_lowTFᵢ where,

counts_newᵢ is the adjusted counts for a bin i for the low-tumor fraction subject (e.g., individual 16) upon spiking with the bin counts from the matched high-tumor fraction subject,

target_TF is the target tumor fraction (x-axis of the graph) for the low-tumor fraction subject (e.g., individual 2813) upon spiking with the bin counts from the matched high-tumor fraction subject,

actual_TF is the actual tumor fraction for the low-tumor fraction subject (e.g., individual 2813) prior to spiking with the bin counts from the matched high-tumor-fraction subject,

counts_highTFᵢ is the bin count for bin i in the matched high-tumor-fraction subject, and

counts_lowTFᵢ is the bin count for bin i in the low-tumor-fraction subject (e.g., individual 2813).

Thus, in this way, each line in the graph of FIG. 7C represents the progressive spiking of a different high-tumor fraction subject into the nucleic acid fragment sequence counts of individual 2813 and thus represents a progression of tumor fraction. As discussed above, for each tumor fraction sampled, for each cancer, the combined allelic counts (e.g., of the individual 2813 with the matched spiked allelic counts) were subjected to a classifier to determine a probability of having the cancer condition (y-axis). In other words, each instance of spiked bin counts (for each line in each graph in FIGS. 12A-12CC) was evaluated by the cancer classifier to generate a probability (y-axis) that the spiked data was acquired from a subject having cancer. These probabilities were plotted as a function of tumor fraction, in the graphs shown in FIGS. 12A-12C.

As shown by the graphs in FIGS. 12A-12C, the probability of cancer calculated for a given simulated sample depended upon (i) the simulated tumor fraction, (ii) the type of cancer, and (iii) the background signal provided by the reference subject (the subject who data was spiked with cancer signal). For instance, referring to reference individual 2813, the plot for which is enlarged in FIGS. 12A-12C, there is a nearly 10-fold difference in the tumor fraction required to generate a spike in the identified cancer probability across the different types of cancers represented by the twenty-two high-tumor fraction subjects. For instance, when signals from a first cancer is spiked into reference individual's 2813 background (represented by series 702), a significant increase in the identified cancer probability is seen at simulated tumor fractions of just greater than 0.001 (0.1%). However, when signal from two other cancers are respectively spiked into the same background (represented by series 704 and 706, respectively), an increase in the identified cancer probability is not seen until the simulated tumor fraction increases above 0.01 (1%). This demonstrates the dependence upon the cancer type on the calculated cancer probability. Similarly, FIGS. 12A-12C shows that the dependence upon the individual's background signal on the calculated cancer probability is rather significant. For instance, in most of the reference backgrounds, a spike-in calculated cancer probability was not observed for one particular cancer type until the tumor fraction of the simulated sample reached above 0.01 (1%). However, when the cancer signal for that cancer was spiked into data for individual 510, a spike-in cancer probability was observed at a tumor fraction significantly below 0.01. In fact, detectable spikes in the calculated cancer probabilities for reference individual 510 were seen significantly earlier for almost all of the different cancer types. In contrast, when the cancer signal for that cancer type was spiked into data for individual 1314, no increase in cancer probability was observed until the tumor fraction rose significantly above 0.01 (1%). In fact, detectable spikes in the calculated cancer probabilities for reference individual 1314 appeared to be significantly delayed for most cancer types.

### Example 2—Overfitting of a Logistic Regression Model

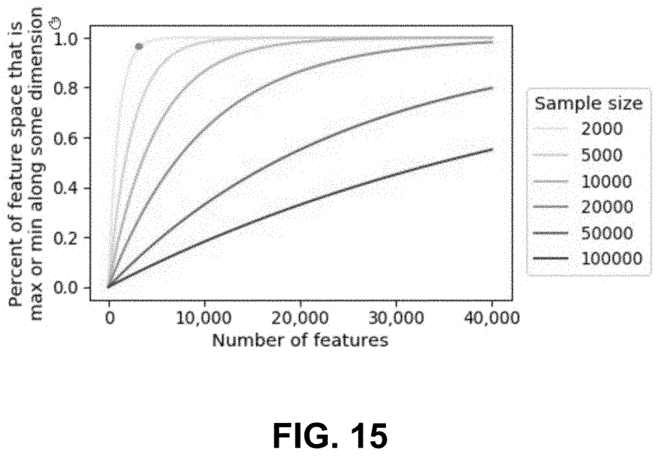

As classification algorithms have become more complex, using larger and larger feature sets, the number of training constructs required for training also expands. In particular, as the number of features used for a disease classifier expands, the number of training constructs having at least one feature value that is an outlier, e.g., present on the surface of a hypercube defining the feature space of the classifier, also expands. This, in turn, leads to overfitting of the classifier and loss of sensitivity, particularly around the level of detection (LOD) for a given disease signal in the classifier. For example, FIG. 15 shows curves representing the percentages of feature space that is maximized or minimized along some dimension as the number of features used in the classifier expands, when the model is trained against 2000 (1002), 5000 (1004), 10,000 (1006), 20,000 (1008), 50,000 (1010), and 100,000 (1012) training constructs. As shown at point 1014, training a classifier with 2500 features with a training set of 2000 samples results in nearly all of the hypercube volume laying on the edge of feature space.

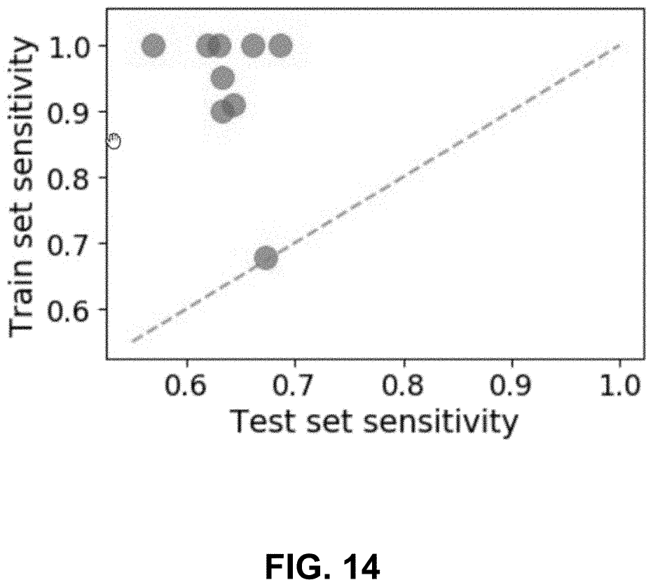

It was observed that machine learning classifiers being trained with thousands of features from the CCGA study described above were being over-fit. It was hypothesized that switching to a more simplistic logistic regression model would solve the problem, as logistic regression is a more rigid model. However, when the same large feature sets were used to train the logistic regression model, the same over-fitting problem was observed. Illustrated in FIG. 14 are the results of nine folds of leave out cross-validation of the logistic regression model. As seen in FIG. 14, the model was over-fit for all but one fold of the cross-validation, as evidenced by the significantly higher sensitivity for the training portion (0.9-1.0) than the test fold (0.6-0.7).

### VI. Claimable Subject Matter

In one aspect, a method for training a model for detecting cancer comprises receiving sequencing data for a plurality of training samples, each training sample labeled as one of cancer and non-cancer and each training sample comprising a plurality of anomalous cfDNA fragments; sampling a first training sample labeled as cancer and a second training sample labeled as non-cancer; generating a first synthetic training sample by sampling a first subset of anomalous cfDNA fragments from the first training sample and a second subset of anomalous cfDNA fragments from the second training sample, the first synthetic training sample labeled as cancer; generating a feature vector for each of the training samples including the first synthetic training sample based on the plurality of anomalous cfDNA fragments of each training sample; and training the model with the feature vectors and the labels of the training samples including the first synthetic training sample, the model configured to generate a cancer prediction for a test sample based on sequencing data of the test sample.

In another aspect, a method for detecting cancer can comprise receiving sequencing data for a test sample comprising a plurality of anomalous cfDNA fragments; generating a test feature vector based on the anomalous cfDNA fragments of the test sample; and inputting the test feature vector into a classification model to generate a cancer prediction for the test sample, wherein the classification model is trained by: receiving sequencing data for a plurality of training samples, each training sample labeled as one of cancer and non-cancer and each training sample comprising a plurality of anomalous cfDNA fragments, sampling a first training sample labeled as cancer and a second training sample labeled as non-cancer, generating a first synthetic training sample by sampling a first subset of anomalous cfDNA fragments from the first training sample and a second subset of anomalous cfDNA fragments from the second training sample, the first synthetic training sample labeled as cancer, generating a feature vector for each of the training samples including the first synthetic training sample based on the plurality of anomalous cfDNA fragments of each training sample, and training the model with the feature vectors and the labels of the training samples including the first synthetic training sample.

In another aspect, the present disclosure provides a method of generating a plurality of supplemental data constructs that facilitate discrimination of a disease condition. The method can include obtaining a training dataset, in electronic form, including a first plurality of genomic data constructs for a first cohort of training subjects having a first state of the disease condition, where the first plurality of genomic data constructs includes, for each respective training subject in the first cohort of training subjects, a respective genomic data construct including values for a plurality of genomic characteristics of a corresponding plurality of nucleic acid fragments in a corresponding biological sample obtained from the respective training subject. The method can then include using the training dataset to generate the plurality of supplemental data constructs, where each respective supplemental genomic data construct in the plurality of supplemental genomic data constructs corresponds to at least a respective genomic data construct from the first plurality of genomic data constructs, and where each respective supplemental genomic data construct in the plurality of supplemental genomic data constructs includes, for each respective genomic characteristic in the plurality of genomic characteristics, an augmented value that is derived from probability sampling of nucleic acid fragments contributing to the value of the respective genomic characteristic in at least the respective genomic data construct from the first plurality of genotypic data constructs.

In some embodiments, the training data set includes a second plurality of genomic data constructs for a second cohort of training subjects having a second state of the disease condition that is different from the first state of the disease condition. The second plurality of genomic data constructs can include, for each respective training subject in the second cohort of training subjects, a respective genomic data construct including values for the plurality of genotypic characteristics of a corresponding plurality of nucleic acid fragments in a corresponding biological sample obtained from the respective training subject. In some embodiments, the method includes training a test classifier to discriminate a state of the disease condition using at least (i) the first plurality of genomic data constructs, (ii) the second plurality of genomic data constructs, (iii) the plurality of supplemental genomic data constructs, and (iv) for each respective genomic data construct in the first plurality of genomic data constructs, second plurality of genomic data constructs, and plurality of supplemental genomic data constructs, an indication of the state of the disease condition.

In another aspect, the disclosure provides a method for discriminating a disease condition in a test subject. The method can include obtaining a test genomic data construct, in electronic form. The test genomic data construct can include a value for each genomic characteristic in a plurality of test genomic characteristics of a corresponding plurality of nucleic acid fragments in a biological sample obtained from the test subject. The method can then include applying the test genomic data construct to a test classifier trained as described above, thereby determining the state of the disease condition in the test subject. In this method, the plurality of test genomic characteristics can include the plurality of genotypic characteristics the test classifier is trained against.

In another aspect, the disclosure provides a method of generating time series data that facilitate discrimination of a disease condition. The method can include obtaining a first training dataset, in electronic form, that includes a first plurality of genomic data constructs for a first cohort of training subjects. The method can then include using the first training data set to generate, for each respective training subject in the first cohort of training subjects, a respective first augmented genomic data construct including values for the plurality of genomic characteristics that are representative of the respective training subject at a respective second time point. The respective first augmented genomic data construct corresponds to a corresponding first pair of genomic data constructs, the first pair of genomic data constructs comprising (i) a respective first genomic data construct for the respective training subject and (ii) a respective spike-in genotypic data construct from the set of one or more spike-in genomic data constructs. The respective first augmented genomic data construct can include, for each respective genomic characteristic in the plurality of genomic characteristics, an augmented value that is derived from a first probability sampling of nucleic acid fragments contributing to the value of the respective genomic characteristic in each genomic data construct of the corresponding first pair of genomic data constructs. The method thereby generates, for each respective training subject in the first cohort of training subjects, a respective time series data set including the respective first genomic data construct and the respective first augmented genomic data construct.

In some embodiments, the method also includes training a temporal classifier to discriminate a state of the disease condition using at least (i) for each respective training subject in the first cohort of training subjects, the respective time series data set, (ii) for each respective training subject in the first cohort of training subjects, a respective plurality of time points including a respective time point for each respective genomic data construct in the respective time series data set, or a derivation thereof, and (iii) for each respective training subject in the first cohort of training subjects, an indication of the disease condition for at least the earliest respective time point and the latest respective time point in the respective plurality of time points.

In one aspect, the disclosure provides a method of training a temporal classification algorithm to discriminate a state of a disease condition of a test subject from among a set of states of the disease condition. The method includes obtaining a training dataset, in electronic form, that includes, for each respective training subject in a plurality of training subjects: (1) a respective first genomic data construct for the respective training subject, the respective first genomic data construct including values for a plurality of genotypic characteristics of a first respective plurality of nucleic acid fragments in a first biological sample obtained from the respective training subject at a respective first time point, (2) a respective second genomic data construct for the respective training subject, the respective second genomic data construct including values for the plurality of genomic characteristics that are representative of the respective training subject at a respective second time point occurring after the respective first time point, (3) the respective first time point and the respective second time point, or a derivation thereof, and (4) an indication of the disease condition in the set of disease conditions, at the respective first time and the respective second time point, of the respective training subject. The method can then include training a temporal classification algorithm against, for each respective training subject, at least (a) the respective first genomic data construct, (b) the respective second genomic data construct, (c) the respective first time point and the respective second time point, or the derivation thereof, and (d) the indication of the disease condition, at the respective first time and the respective second time point. For at least one respective training subject in the plurality of training subjects, the respective second genomic data construct can include values for the plurality of genomic characteristics from a respective second plurality of nucleic acid fragments from a second biological sample obtained from the respective training subject and a respective third plurality of nucleic acid fragments from a spike-in biological sample obtained from a spike-in subject afflicted with a respective state of the disease condition in the set of states of the disease condition.

In another aspect, the disclosure provides a method for discriminating a disease condition in a test subject. The method can include obtaining a test time series data set, in electronic form, for a test subject. The test time series data set can include, (i) for each respective time point in a plurality of time points, a respective test genomic data construct including values for a plurality of test genomic characteristics of a corresponding plurality of nucleic acid fragments in a corresponding biological sample obtained from the test subject at the respective time point, and (ii) for each respective pair of consecutive time points in the plurality of time points, an indication of the length of time between the respective pair of consecutive time points. The method can then include applying the test time series data set to a classifier trained as described above, thereby determining the state of the disease condition in the test subject. In this method, the plurality of test genomic characteristics includes the plurality of genomic characteristics the classifier was trained against.

In one aspect, the disclosure provides a method of assessing the performance of a classifier trained to discriminate a disease condition in a test subject. The method can include obtaining a first classifier trained to discriminate a disease condition by evaluating a test genomic data construct, where the test genomic data construct includes values for a plurality of genomic characteristics of a corresponding first plurality of nucleic acid fragments in a first corresponding biological sample obtained from the test subject. The method can then include obtaining an augmented assessment data set including a plurality of augmented genomic data constructs. Each respective augmented genotypic data construct in the plurality of augmented genotypic data constructs can include values for the plurality of genomic characteristics of a corresponding plurality of nucleic acid fragments representative of a corresponding biological sample obtained from a subject having a respective state of the disease condition in a plurality of states of the disease condition. The augmented assessment data set can include respective augmented genomic data constructs, in the plurality of augmented genotypic data constructs, representative of each respective state of the disease condition in the plurality of states of the disease condition. The method can further include independently applying each respective augmented genomic data construct in the augmented assessment data set to the classifier to generate a disease state classification for each respective augmented genotypic data construct, thereby generating a plurality of disease state classifications. The method can then include evaluating each respective disease state classification, in the plurality of disease state classifications, as a function of the respective state of the disease condition represented by the corresponding augmented genomic data construct, thereby assessing the performance of the classifier.

Another aspect of the disclosure provides a method of generating a plurality of supplemental data constructs that facilitate discrimination of a disease condition, the method comprising: at a computer system comprising at least one processor and a memory storing at least one program for execution by the at least one processor, the at least one program comprising instructions for: A) obtaining a training dataset, in electronic form, comprising: a first plurality of genotypic data constructs for a first cohort of training subjects having a first state of the disease condition, wherein the first plurality of genotypic data constructs includes, for each respective training subject in the first cohort of training subjects, a respective genotypic data construct comprising values for a plurality of genotypic characteristics of a corresponding plurality of nucleic acid fragments in a corresponding biological sample obtained from the respective training subject; and B) using the training dataset to generate the plurality of supplemental data constructs, wherein each respective supplemental genotypic data construct in the plurality of supplemental genotypic data constructs corresponds to at least a respective genotypic data construct from the first plurality of genotypic data constructs, and wherein each respective supplemental genotypic data construct in the plurality of supplemental genotypic data constructs comprises, for each respective genotypic characteristic in the plurality of genotypic characteristics, an augmented value that is derived from probability sampling of nucleic acid fragments contributing to the value of the respective genotypic characteristic in at least the respective genotypic data construct from the first plurality of genotypic data constructs.

In some embodiments, the training data set further comprises a second plurality of genotypic data constructs for a second cohort of training subjects having a second state of the disease condition that is different from the first state of the disease condition, wherein the second plurality of genotypic data constructs includes, for each respective training subject in the second cohort of training subjects, a respective genotypic data construct comprising values for the plurality of genotypic characteristics of a corresponding plurality of nucleic acid fragments in a corresponding biological sample obtained from the respective training subject, the method further comprising: C) training a test classifier to discriminate a state of the disease condition using at least (i) the first plurality of genotypic data constructs, (ii) the second plurality of genotypic data constructs, (iii) the plurality of supplemental genotypic data constructs, and (iv) for each respective genotypic data construct in the first plurality of genotypic data constructs, second plurality of genotypic data constructs, and plurality of supplemental genotypic data constructs, an indication of the state of the disease condition.

In some embodiments, the training C) uses a third plurality of genotypic data constructs for a third cohort of training subjects, wherein the third plurality of genotypic data constructs includes, for each respective training subject in the third cohort of training subjects, a respective genotypic data construct comprising values for the plurality of genotypic characteristics of a corresponding plurality of nucleic acid fragments in a corresponding biological sample obtained from the respective training subject, wherein each training subject in the third cohort has a third state of the disease condition.

In some embodiments, the training C) uses one or more personal characteristics of the respective training subject.

In some embodiments, the disease condition is cancer.

In some embodiments, the first state of the cancer is a presence of the cancer and a second state of the cancer is an absence of the cancer.

In some embodiments, the first state of the cancer is a first type of cancer and a second state of the cancer is a second type of cancer.

In some embodiments, the first state of the cancer is a first stage of a specified cancer and a second state of the cancer is a second stage of the specified cancer.

In some embodiments, the first state of the cancer is a first prognosis for the cancer and a second state of the cancer is a second prognosis for the cancer.

In some embodiments, the disease condition is a cardiovascular disease.

In some embodiments, the first state of the cardiovascular disease is a presence of the cardiovascular disease and a second state of the cardiovascular disease is an absence of the cardiovascular disease.

In some embodiments, the first state of the cardiovascular disease is a first prognosis for the cardiovascular disease and a second state of the cardiovascular disease is a second prognosis for the cardiovascular disease.

In some embodiments, the plurality of genotypic characteristics comprises, for each respective genomic location in a plurality of genomic locations, a methylation status of the respective genomic location.

In some embodiments, the plurality of genotypic characteristics comprises, for each respective genomic location in a plurality of genomic locations, support for a variant allele.

In some embodiments, the plurality of genotypic characteristics comprises, for each respective genomic location in a plurality of genomic locations, a relative copy number.

In some embodiments, the plurality of genotypic characteristics comprises at least 5000 genotypic characteristics.

In some embodiments, the plurality of genotypic characteristics comprises at least 50,000 genotypic characteristics.

In some embodiments, the training dataset comprises fewer than 20,000 genotypic data constructs.

In some embodiments, the training dataset comprises fewer than 2000 genotypic data constructs.

In some embodiments, for each biological sample, the values for the plurality of genotypic characteristics of the corresponding plurality of nucleic acid fragments are obtained by whole-genome sequencing.

In some embodiments, for each biological sample, the values for the plurality of genotypic characteristics of the corresponding plurality of nucleic acid fragments are obtained by targeted sequencing using a plurality of nucleic acid probes to enrich nucleic acids in the corresponding biological sample for a plurality of genomic regions.

In some embodiments, for each biological sample, the values for the plurality of genotypic characteristics of the corresponding plurality of nucleic acid fragments are obtained by (i) whole-genome methylation sequencing or (ii) targeted DNA methylation sequencing using a plurality of nucleic acid probes to enrich nucleic acids in the corresponding biological sample for a plurality of genomic regions.

In some embodiments, each corresponding biological sample is a liquid biological sample.

In some embodiments, the liquid biological sample is a blood sample.

In some embodiments, the plurality of nucleic acid fragments in the corresponding biological sample are cell-free DNA.

In some embodiments, the probability sampling is simple random sampling, stratified random sampling, systematic random sampling, clustered random sampling, or multi-stage random sampling.

In some embodiments, the probability sampling comprises weighted random sampling of a predetermined portion of the plurality of nucleic acid fragments contributing to the values of the plurality of genotypic characteristics, wherein the probability of selecting a respective nucleic acid fragment that contributes to the value of a corresponding genotypic characteristic is proportional to the abundance of nucleic acid fragments contributing the corresponding genotypic characteristic relative to the total number of nucleic acid fragments contributing to the values of the plurality of genotypic characteristics.

In some embodiments, for each respective supplemental data construct in the plurality of supplemental data constructs: the probability sampling selects a respective portion of the plurality of nucleic acid fragments that contribute to the values for the plurality of genotypic characteristics in the respective data construct from the first plurality of genotypic data constructs; and the magnitude of the respective portion of nucleic acid fragments is determined independently from the magnitudes of the respective portions of nucleic acid fragments selected for the other supplemental data constructs.

In some embodiments, for each respective supplemental data construct in the plurality of supplemental data constructs: the probability sampling selects a respective portion of the plurality of nucleic acid fragments that contribute to the values for the plurality of genotypic characteristics in the respective data construct from the first plurality of genotypic data constructs; and the magnitude of the respective portion of nucleic acid fragments is selected such that the respective supplemental data construct represents a simulated informative nucleic acid fragment fraction falling within a range of informative nucleic acid fragment fractions over which an exploratory classifier satisfies a threshold sensitivity to changes in the informative nucleic acid fragment fraction represented by the genotypic data construct, wherein the exploratory classifier is trained to discriminate a state of the disease condition based on the plurality of genotypic characteristics.

In some embodiments, the range of informative nucleic acid fragment fractions is determined by: a) using the training dataset to generate a plurality of augmented exploratory genotypic data constructs, wherein: each respective augmented exploratory genotypic data construct in the plurality of augmented exploratory genotypic data constructs corresponds to at least a respective genotypic data construct from the first plurality of genotypic data constructs, each respective augmented exploratory genotypic data construct in the plurality of augmented exploratory genotypic data constructs comprises, for each respective genotypic characteristic in the plurality of genotypic characteristics, an augmented value that is derived from probability sampling of nucleic acid fragments contributing to the value of the respective genotypic characteristic from at least the respective genotypic data construct from the first plurality of genotypic data constructs; each respective augmented exploratory genotypic data construct in the plurality of augmented exploratory genotypic data constructs represents a simulated informative nucleic acid fragment fraction that is based upon the informative nucleic acid fragment fraction represented by the respective genotypic data construct from the first plurality of genotypic data constructs; and the distribution of simulated informative nucleic acid fragment fractions represented by the plurality of augmented exploratory genotypic data constructs spans from a first informative nucleic acid fragment fraction that is below a level of detection for an exploratory classifier to a second informative nucleic acid fragment fraction that is above the level of detection for the exploratory classifier; b) applying the plurality of augmented exploratory genotypic data constructs to the exploratory classifier to generate a plurality of simulated disease condition probabilities, wherein the exploratory classifier was trained to discriminate a state of the disease condition using at least: (1) a first plurality of exploratory genotypic data constructs, wherein the first plurality of exploratory genotypic data constructs includes, for each respective exploratory subject in a first cohort of exploratory subjects that have the first state of the disease condition, a respective genotypic data construct comprising values for the plurality of genotypic characteristics of a corresponding plurality of nucleic acid fragments in a corresponding biological sample obtained from the respective exploratory subject, (2) a second plurality of exploratory data constructs, wherein the second plurality of exploratory genotypic data constructs includes, for each respective exploratory subject in a second cohort of exploratory subjects that have a second state of the disease condition, a respective genotypic data construct comprising values for the plurality of genotypic characteristics of a corresponding plurality of nucleic acid fragments in a corresponding biological sample obtained from the respective exploratory subject, and (3) for each respective genotypic data construct in the first and second pluralities of exploratory genotypic data constructs, an indication of the state of the disease condition; and c) identifying the range of informative nucleic acid fragment fractions over which simulated disease condition probabilities are most sensitive to changes in the informative nucleic acid fragment fraction represented by a respective augmented exploratory genotypic data construct.

In some embodiments, each respective biological sample obtained from an exploratory subject in the first cohort of exploratory subjects is a sample of a solid, diseased tissue of the subject.

In some embodiments, each respective supplemental genotypic data construct in the plurality of supplemental genotypic data constructs corresponds to a corresponding pair of genotypic constructs, the pair of genotypic constructs consisting of (i) the respective genotypic data construct from the first plurality of genotypic data constructs and (ii) a respective genotypic data construct from the second plurality of genotypic data constructs; and each respective supplemental genotypic data construct in the supplemental plurality of genotypic data constructs comprises, for each respective genotypic characteristic in the plurality of genotypic characteristics, an augmented value that is derived from a probability sampling of nucleic acid fragments contributing to the value of the respective genotypic characteristic in each genotypic data construct of the corresponding pair of genotypic data constructs.

In some embodiments, for at least one respective supplemental genotypic data construct in the plurality of supplemental genotypic data constructs, the respective genotypic data construct from the second plurality of genotypic data constructs is augmented prior to deriving the augmented values for the plurality of genotypic characteristics of the respective supplemental genotypic data construct.

In some embodiments, for each respective supplemental genotypic data construct in the plurality of supplemental genotypic data constructs, the augmented value for each respective genotypic characteristic in the plurality of genotypic characteristics is formed from (i) a first weighted contribution of the respective genotypic characteristic from the respective genotypic data construct from the first plurality of genotypic data constructs, and (ii) a second weighted contribution of the respective genotypic characteristic from the respective genotypic data construct from the second plurality of genotypic data constructs.

In some embodiments, for each respective supplemental genotypic data construct in the plurality of supplemental genotypic data constructs, the (i) respective training subject corresponding to the respective genotypic data construct from the first plurality of genotypic data constructs and (ii) respective training subject corresponding to the respective genotypic data construct from the second plurality of genotypic data constructs, corresponding to the pair of genotypic constructs, are matched based on a shared personal characteristic.

In some embodiments, the method further comprises: obtaining a plurality of augmented false-positive genotypic data constructs by: identifying a subset of genotypic data constructs from the second plurality of genotypic data constructs that are discriminated by a precursor to the test classifier with a performance that fails a performance threshold; and using the subset of genotypic data constructs to generate the plurality of augmented false-positive genotypic data constructs, wherein: each respective augmented false-positive genotypic data construct in the plurality of augmented false-positive genotypic data constructs corresponds to at least a respective genotypic data construct from the sub-set of genotypic data constructs, and each respective genotypic data construct in the plurality of augmented false-positive genotypic data constructs comprises, for each respective genotypic characteristic in the plurality of genotypic characteristics, an augmented value that is derived from probability sampling of nucleic acid fragments contributing to the value of the respective genotypic characteristic in at least the respective genotypic data construct from the sub-set of genotypic data constructs, wherein training the test classifier (C) uses (v) the plurality of augmented false-positive genotypic data constructs, and (vi) for each respective genotypic data construct in the plurality of augmented false-positive genotypic data constructs, an indication of the state of the disease condition.

In some embodiments, the test classifier is a logistic regression algorithm.

In some embodiments, the test classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

In some embodiments, the test classifier is a temporal classifier that requires at least (i) a first test genotypic data construct generated from a first biological sample acquired from a test subject at a first point in time, and (ii) a second test genotypic data construct generated from a second biological sample acquired from a test subject at a second point in time.

In some embodiments, the method further comprises: D) obtaining a test genotypic data construct, in electronic form, that includes a value for each genotypic characteristic in the plurality of genotypic characteristics of a corresponding plurality of nucleic acid fragments in a biological sample obtained from a test subject, and E) applying the test genotypic data construct to the test classifier to thereby determine the state of the disease condition in the test subject.

In some embodiments, the test subject was not previously diagnosed with the disease condition, prior to the applying E).

Another aspect of the disclosure provides a method for discriminating a disease condition in a test subject, the method comprising: at a computer system comprising at least one processor and a memory storing at least one program for execution by the at least one processor, the at least one program comprising instructions for: A) obtaining a test genotypic data construct, in electronic form, that includes a value for each genotypic characteristic in a plurality of test genotypic characteristics of a corresponding plurality of nucleic acid fragments in a biological sample obtained from the test subject; and B) applying the test genotypic data construct to a test classifier trained according to a method of any one of claims 2-39 to thereby determine the state of the disease condition in the test subject, wherein the plurality of test genotypic characteristics comprises the plurality of genotypic characteristics the test classifier was trained against.

In some embodiments, the biological sample obtained from the test subject is a liquid biological sample.

In some embodiments, the liquid biological sample is a blood sample.

In some embodiments, the plurality of nucleic acid fragments in the biological sample obtained from the test subject are cell-free DNA.

Another aspect of the disclosure provides a method of generating time series data that facilitate discrimination of a disease condition, the method comprising: at a computer system comprising at least one processor and a memory storing at least one program for execution by the at least one processor, the at least one program comprising instructions for: A) obtaining a first training dataset, in electronic form, that comprises: a first plurality of genotypic data constructs for a first cohort of training subjects, wherein the first plurality of genotypic data constructs includes, for each respective training subject in the first cohort of training subjects, a respective first genotypic data construct comprising values for a plurality of genotypic characteristics of a corresponding first plurality of nucleic acid fragments in a corresponding first biological sample obtained from the respective training subject at a respective first time point, wherein the respective training subject has a first state of the disease condition at the respective first time point, and a set of one or more spike-in genotypic data constructs for a cohort of one or more spike-in subjects, wherein the set of one or more spike-in genotypic data constructs includes, for each respective spike-in subject in the set or one or more spike-in subjects, a respective spike-in genotypic data construct comprising values for the plurality of genotypic characteristics of a corresponding plurality of nucleic acid fragments in a corresponding biological sample obtained from the respective spike-in subject, wherein the respective spike-in subject had a second state of the disease condition when the corresponding biological sample was obtained from the respective spike-in subject, and wherein the first state of the disease condition and the second state of the disease condition are related by progression of the disease condition; and B) using the first training data set to generate, for each respective training subject in the first cohort of training subjects, a respective first augmented genotypic data construct comprising values for the plurality of genotypic characteristics that are representative of the respective training subject at a respective second time point, wherein: the respective first augmented genotypic data construct corresponds to a corresponding first pair of genotypic data constructs, the first pair of genotypic data constructs consisting of (i) a respective second genotypic data construct for the respective training subject and (ii) a respective spike-in genotypic data construct from the set of one or more spike-in genotypic data constructs; and the respective first augmented genotypic data construct comprises, for each respective genotypic characteristic in the plurality of genotypic characteristics, an augmented value that is derived from a first probability sampling of nucleic acid fragments contributing to the value of the respective genotypic characteristic in each genotypic data construct of the corresponding first pair of genotypic data constructs, thereby generating, for each respective training subject in the first cohort of training subjects, a respective time series data set comprising the respective first genotypic data construct and the respective first augmented genotypic data construct.

In some embodiments, for at least one respective training subject in the first cohort of training subjects, the respective second genotypic data construct is the respective first genotypic data construct.

In some embodiments, for at least one respective training subject in the first cohort of training subjects, the respective second genotypic data construct comprises values for the plurality of genotypic characteristics of a corresponding second plurality of nucleic acid fragments in a corresponding second biological sample obtained from the respective training subject at the second time point.

In some embodiments, the method further comprises: using the first training data set to generate, for each respective training subject in the first cohort of training subjects, a respective second augmented genotypic data construct comprising values for the plurality of genotypic characteristics that are representative of the respective training subject at a respective third time point, wherein: the respective second augmented genotypic data construct corresponds to a corresponding second pair of genotypic constructs consisting of (i) a respective third genotypic data construct for the respective training subject and (ii) the respective spike-in genotypic data construct from the set of one or more spike-in genotypic data constructs; and the respective second augmented genotypic data construct comprises, for each respective genotypic characteristic in the plurality of genotypic characteristics, an augmented value that is derived from a second probability sampling of nucleic acid fragments contributing to the value of the respective genotypic characteristic in each genotypic data construct of the corresponding second pair of genotypic data constructs, thereby expanding, for each respective training subject in the first cohort of training subjects, the respective time series data set by inclusion of the respective second augmented genotypic data construct.

In some embodiments, for at least one respective training subject in the first cohort of training subjects, the respective third genotypic data construct is the respective first genotypic data construct.

In some embodiments, for at least one respective training subject in the first cohort of training subjects: the respective second genotypic data construct comprises values for the plurality of genotypic characteristics of a corresponding second plurality of nucleic acid fragments in a corresponding second biological sample obtained from the respective training subject at the second time point; and the respective third genotypic data construct is the respective second genotypic data construct.

In some embodiments, for at least one respective training subject in the first cohort of training subjects, the respective third genotypic data construct comprises values for the plurality of genotypic characteristics of a corresponding third plurality of nucleic acid fragments in a third corresponding biological sample obtained from the respective training subject at the third time point.

In some embodiments, for each respective training subject in the first cohort of training subjects: the respective first time series data set is for modeling development of the second disease state from the first disease state; the second time point corresponds to a time point that is after the first time point; the third time point corresponds to a time point that is after the second time point; and the second probability sampling of nucleic acid fragments is weighted more heavily towards selection of nucleic acid fragments contributing to the value of the genotypic characteristics in the respective spike-in genotypic data construct than is the first probability sampling.

In some embodiments, the method further comprises: C) training a temporal classifier to discriminate a state of the disease condition using at least (i) for each respective training subject in the first cohort of training subjects, the respective time series data set, (ii) for each respective training subject in the first cohort of training subjects, a respective plurality of time points comprising a respective time point for each respective genotypic data construct in the respective time series data set, or a derivation thereof, and (iii) for each respective training subject in the first cohort of training subjects, an indication of the disease condition for at least the earliest respective time point and the latest respective time point in the respective plurality of time points.

In some embodiments, the training C) uses one or more personal characteristics of the respective training subject.

In some embodiments, for at least one respective training subject in the first cohort of training subjects: the respective spike-in genotypic data construct in the respective pair of genotypic data constructs comprises values for the plurality of genotypic characteristics of the corresponding plurality of nucleic acid fragments in the corresponding biological sample obtained from the respective training subject at a fourth corresponding time point that is after the first corresponding time point; and the second corresponding time point is between the first corresponding time point and the fourth corresponding time point.

In some embodiments, for each respective training subject in the first cohort of training subjects, the spike-in subject corresponding to the respective spike-in genotypic data construct in the corresponding pair of genotypic data constructs is a different subject than the respective training subject.

In some embodiments, for each respective training subject in the first cohort of training subjects, the spike-in subject corresponding to the respective spike-in genotypic data construct in the corresponding pair of genotypic data constructs is matched to the respective training subject based on a shared personal characteristic.

In some embodiments, the disease condition is cancer.

In some embodiments, the first state of the cancer is a presence of the cancer and the second state of the cancer is an absence of the cancer.

In some embodiments, the first state of the cancer is a first type of cancer and the second state of the cancer is a second type of cancer.

In some embodiments, the first state of the cancer is a first stage of a specified cancer and the second state of the cancer is a second stage of the specified cancer.

In some embodiments, the first state of the cancer is a first prognosis for the cancer and the second state of the cancer is a second prognosis for the cancer.

In some embodiments, for at least one respective training subject in the first cohort of training subjects: the respective training subject was not afflicted with cancer at the respective first time point, and the respective spike-in genotypic data construct in the pair of genotypic data constructs was obtained from a corresponding spike-in subject who was afflicted with at least stage 2 cancer when the corresponding biological sample was obtained from the respective spike-in subject.

In some embodiments, the disease condition is a cardiovascular disease.

In some embodiments, the first state of the cardiovascular disease is a presence of the cardiovascular disease and the second state of the cardiovascular disease is an absence of the cardiovascular disease.

In some embodiments, the first state of the cardiovascular disease is a first prognosis for the cardiovascular disease and the second state of the cardiovascular disease is a second prognosis for the cardiovascular disease.

In some embodiments, for at least one respective training subject in the first cohort of training subjects: the respective training subject was not afflicted with the cardiovascular disease at the respective first time point, and the respective spike-in genotypic data construct in the pair of genotypic data constructs was obtained from a corresponding spike-in subject who was afflicted with the cardiovascular disease when the corresponding biological sample was obtained from the respective spike-in subject.

In some embodiments, the plurality of genotypic characteristics comprises, for each respective genomic location in a plurality of genomic locations, a methylation status of the respective genomic location.

In some embodiments, the plurality of genotypic characteristics comprises, for each respective genomic location in a plurality of genomic locations, support for a variant allele.

In some embodiments, the plurality of genotypic characteristics comprises, for each respective genomic location in a plurality of genomic locations, a relative copy number.

In some embodiments, the plurality of genotypic characteristics comprises at least 5000 genotypic characteristics.

In some embodiments, the plurality of genotypic characteristics comprises at least 50,000 genotypic characteristics.

In some embodiments, for each biological sample, the values for the plurality of genotypic characteristics of the corresponding plurality of nucleic acid fragments are obtained by whole-genome sequencing.

In some embodiments, for each biological sample, the values for the plurality of genotypic characteristics of the corresponding plurality of nucleic acid fragments are obtained by targeted sequencing using a plurality of nucleic acid probes to enrich nucleic acids in the corresponding biological sample for a plurality of genomic regions.

In some embodiments, for each biological sample, the values for the plurality of genotypic characteristics of the corresponding plurality of nucleic acid fragments are obtained by (i) whole-genome methylation sequencing or (ii) targeted DNA methylation sequencing using a plurality of nucleic acid probes to enrich the nucleic acids in the corresponding biological sample for a plurality of genomic regions.

In some embodiments, for each respective training subject in a plurality of training subjects, the first corresponding biological sample is a liquid biological sample.

In some embodiments, the liquid biological sample is a blood sample.

In some embodiments, the plurality of nucleic acid fragments in the corresponding biological sample are cell-free DNA.

In some embodiments, the probability sampling is simple random sampling, stratified random sampling, systematic random sampling, clustered random sampling, or multi-stage random sampling.

In some embodiments, the probability sampling comprises, for each respective genotypic data construct in each respective pair of genotypic data constructs, weighted random sampling of a predetermined portion of the corresponding plurality of nucleic acid fragments contributing to the corresponding values of the plurality of genotypic characteristics, wherein the probability of selecting a respective nucleic acid fragment that contributes to the value of a corresponding genotypic characteristic is proportional to the abundance of nucleic acid fragments contributing the corresponding genotypic characteristic relative to the total number of nucleic acid fragments contributing to the values of the plurality of genotypic characteristics.

In some embodiments, for the second respective genotypic data construct corresponding to each respective training subject in the first cohort of training subjects: the probability sampling selects a respective first portion of the plurality of nucleic acid fragments that contribute to the values for the plurality of genotypic characteristics in the first respective genotypic construct and a respective second portion of the plurality of nucleic acid fragments that contribute to the values for the plurality of genotypic characteristics in the respective spike-in genotypic data construct; and the magnitude of the respective first portion of nucleic acid fragments and the respective second portion of nucleic acid fragments is determined based on at least (i) the length of time between the first time point and the second time point and (ii) a temporal model for development of the second state of the disease condition from the first state of the disease condition.

In some embodiments, the temporal model for development of the second state of the disease condition from the first state of the disease condition is based at least on a personal characteristic of the respective subject.

In some embodiments, the disease condition is cancer, and the temporal model for development of the second state of the cancer from the first state of the cancer is based at least on the type of cancer.

In some embodiments, the disease condition is cancer, and the temporal model for development of the second state of the cancer from the first state of the cancer is based at least on whether the cancer is metastatic or non-metastatic.

In some embodiments, the disease condition is cancer, and the temporal model for development of the second state of the cancer from the first state of the cancer is separated into stages.

In some embodiments, the temporal classifier is a logistic regression algorithm.

In some embodiments, the temporal classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

In some embodiments, the temporal classifier is a recurrent neural network.

In some embodiments, the method further comprises: D) obtaining a test time series data set, in electronic form, for a test subject, wherein the test time series data set comprises: for each respective time point in a plurality of time points, a corresponding test genotypic data construct comprising values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points; and E) applying the test time series data set to the temporal classifier to thereby determine the state of the disease condition in the test subject.

In some embodiments, the test subject was not previously diagnosed with the disease condition, prior to the applying E).

Another aspect of the present disclosure provides a method for discriminating a disease condition in a test subject, the method comprising: at a computer system comprising at least one processor and a memory storing at least one program for execution by the at least one processor, the at least one program comprising instructions for: A) obtaining a test time series data set, in electronic form, for a test subject, wherein the test time series data set comprises: for each respective time point in a plurality of time points, a respective test genotypic data construct comprising values for a plurality of test genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points; and B) applying the test time series data set to a classifier trained according to one of the training methods to thereby determine the state of the disease condition in the test subject, wherein the plurality of test genotypic characteristics comprises the plurality of genotypic characteristics the classifier was trained against.

In some embodiments, each respective biological sample obtained from the test subject is a liquid biological sample.

In some embodiments, the liquid biological sample is a blood sample.

In some embodiments, the nucleic acid fragments in each biological sample obtained from the test subject are cell-free DNA.

Another aspect of the present disclosure provides a method of training a temporal classification algorithm to discriminate a state of a disease condition of a test subject from among a set of states of the disease condition, the method comprising: at a computer system comprising at least one processor and a memory storing at least one program for execution by the at least one processor, the at least one program comprising instructions for: A) obtaining a training dataset, in electronic form, that comprises, for each respective training subject in a plurality of training subjects: (1) a respective first genotypic data construct for the respective training subject, the respective first genotypic data construct comprising values for a plurality of genotypic characteristics of a first respective plurality of nucleic acid fragments in a first biological sample obtained from the respective training subject at a respective first time point, (2) a respective second genotypic data construct for the respective training subject, the respective second genotypic data construct comprising values for the plurality of genotypic characteristics that are representative of the respective training subject at a respective second time point occurring after the respective first time point, (3) the respective first time point and the respective second time point, or a derivation thereof, and (4) an indication of the disease condition in the set of disease conditions, at the respective first time and the respective second time point, of the respective training subject; and B) training a temporal classification algorithm against, for each respective training subject, at least (a) the respective first genotypic data construct, (b) the respective second genotypic data construct, (c) the respective first time point and the respective second time point, or the derivation thereof, and (d) the indication of the disease condition, at the respective first time and the respective second time point, wherein, for at least one respective training subject in the plurality of training subjects, the respective second genotypic data construct comprises values for the plurality of genotypic characteristics from a respective second plurality of nucleic acid fragments from a second biological sample obtained from the respective training subject and a respective third plurality of nucleic acid fragments from a spike-in biological sample obtained from a spike-in subject afflicted with a respective state of the disease condition in the set of states of the disease condition.

In some embodiments, the training B) uses one or more personal characteristics of the respective training subject.

In some embodiments, the training data set further includes, for each respective training subject in the plurality of training subjects: (5) a respective third genotypic data construct for the respective training subject, the respective third genotypic data construct comprising values for the plurality of genotypic characteristics that are representative of the respective training subject at a respective third time point occurring after the respective second time point, (6) the respective third time point, or a derivation of the respective second time point and the respective third time point, and (7) an indication of the state of the disease condition in the set of states of the disease condition, at the respective third time point, of the respective training subject; the temporal classification algorithm is further trained against (b1) the respective third genotypic data construct, (c1) the respective third time point, or the derivation of the respective second time point and the respective second time point, and (d1) the indication of the state of the disease condition in the set of states of the disease condition, at the respective third time point, of the respective training subject; and wherein, for at least one respective training subject in the plurality of training subjects, the respective third genotypic data construct comprises values for the plurality of genotypic characteristics from a respective fourth plurality of nucleic acid fragments from a third biological sample obtained from the respective training subject and a respective fifth plurality of nucleic acid fragments from a spike-in biological sample obtained from a spike-in subject with the respective state of the disease condition in the set of states of the disease condition.

In some embodiments, the respective second plurality of nucleic acid fragments and the respective fourth plurality of nucleic acid fragments are the same cell-free nucleic acids from the same biological sample obtained from the respective training subject.

In some embodiments, the respective third plurality of nucleic acid fragments and the respective fifth plurality of nucleic acid fragments are the same cell-free nucleic acids from the same spike-in biological sample obtained from the spike-in subject.

In some embodiments, the respective third plurality of nucleic acid fragments and the respective fifth plurality of nucleic acid fragments are the same cell-free nucleic acids from the same spike-in biological sample obtained from the spike-in subject; the values for the plurality of genotypic characteristics in the respective second genotypic data construct comprise a respective first weighted mixture of (i) values for the plurality of genotypic characteristics of the respective second plurality of nucleic acid fragments and (ii) values for the plurality of genotypic characteristics of the respective third plurality of nucleic acid fragments; the values for the plurality of genotypic characteristics in the respective third genotypic data construct comprise a respective second weighted mixture of (i) values for the plurality of genotypic characteristics of the respective second plurality of nucleic acid fragments and (ii) values for the plurality of genotypic characteristics of the respective third plurality of nucleic acid fragments; and the respective second weighted mixture is weighted more heavily towards the values for the plurality of the genotypic characteristics of the respective third plurality of nucleic acid fragments than is the respective first weighted mixture.

In some embodiments, for a respective training subject of the at least one respective training subject, the respective third plurality of nucleic acid fragments are cell-free nucleic acids in a biological sample obtained from the respective training subject at a respective third time point occurring after the respective second time point.

In some embodiments, for a respective training subject of the at least one respective training subject, the spike-in subject is a different subject than the respective training subject.

In some embodiments, for a respective training subject of the at least one respective training subject, the spike-in subject is matched to the respective training subject based on a shared personal characteristic.

In some embodiments, the disease condition is cancer.

In some embodiments, a first state, in the set of states, of the cancer is a presence of the cancer and a second state, in the set of states, of the cancer is an absence of the cancer.

In some embodiments, a first state, in the set of states, of the cancer is a first type of cancer and a second state, in the set of states, of the cancer is a second type of cancer.

In some embodiments, a first state, in the set of states, of the cancer is a first stage of a specified cancer and a second state, in the set of states, of the cancer is a second stage of the specified cancer.

In some embodiments, a first state, in the set of states, of the cancer is a first prognosis for the cancer and a second state, in the set of states, of the cancer is a second prognosis for the cancer.

In some embodiments, for at least one respective training subject in the first cohort of training subjects: the respective training subject was not afflicted with cancer at the respective first time point; the respective second genotypic data construct for the respective training subject is representative of the respective training subject having cancer at the respective second time point; and the spike-in subject was afflicted with at least stage 2 cancer when the spike-in biological sample was obtained.

In some embodiments, the disease condition is a cardiovascular disease.

In some embodiments, a first state, in the set of states, of the cardiovascular disease is a presence of the cardiovascular disease and a second state, in the set of states, of the cardiovascular disease is an absence of the cardiovascular disease.

In some embodiments, a first state, in the set of states, of the cardiovascular disease is a first prognosis for the cardiovascular disease and a second state, in the set of states, of the cardiovascular disease is a second prognosis for the cardiovascular disease.

In some embodiments, for at least one respective training subject in the first cohort of training subjects: the respective training subject was not afflicted with the cardiovascular disease at the respective first time point; and the respective second genotypic data construct for the respective training subject is representative of the respective training subject having cardiovascular disease at the respective second time point; and the spike-in subject was afflicted with cardiovascular disease when the spike-in biological sample was obtained.

In some embodiments, the plurality of genotypic characteristics comprises, for each respective genomic location in a plurality of genomic locations, a methylation status of the respective genomic location.

In some embodiments, the plurality of genotypic characteristics comprises, for each respective genomic location in a plurality of genomic locations, support for a variant allele.

In some embodiments, the plurality of genotypic characteristics comprises, for each respective genomic location in a plurality of genomic locations, a relative copy number.

In some embodiments, the plurality of genotypic characteristics comprises at least 5000 genotypic characteristics.

In some embodiments, the plurality of genotypic characteristics comprises at least 50,000 genotypic characteristics.

In some embodiments, for each biological sample, the values for the plurality of genotypic characteristics of the corresponding plurality of nucleic acid fragments are obtained by whole-genome sequencing.

In some embodiments, for each biological sample, the values for the plurality of genotypic characteristics of the corresponding plurality of nucleic acid fragments are obtained by targeted sequencing using a plurality of nucleic acid probes to enrich the nucleic acids in the corresponding biological sample for a plurality of genomic regions.

In some embodiments, for each biological sample, the values for the plurality of genotypic characteristics of the corresponding plurality of nucleic acid fragments are obtained by (i) whole-genome methylation sequencing or (ii) targeted DNA methylation sequencing using a plurality of nucleic acid probes to enrich the nucleic acids in the corresponding biological sample for a plurality of genomic regions.

In some embodiments, each biological sample is a liquid biological sample.

In some embodiments, the liquid biological sample is a blood sample.

In some embodiments, the plurality of nucleic acid fragments in the corresponding biological sample are cell-free DNA.

In some embodiments, for each respective training subject of the at least one respective training subject, the respective second genotypic data construct comprises, for each respective genotypic characteristic in the plurality of genotypic characteristics, an augmented value that is derived from a probability sampling of (i) nucleic acid fragments contributing to the value of the respective genotypic characteristic in the second plurality of nucleic acid fragments, and (ii) nucleic acid fragments contributing to the value of the respective genotypic characteristic in the third plurality of nucleic acid fragments.

In some embodiments, the probability sampling is simple random sampling, stratified random sampling, systematic random sampling, clustered random sampling, or multi-stage random sampling.

In some embodiments, the probability sampling comprises, for each of the respective second plurality of nucleic acid fragments and the respective third plurality of nucleic acid fragments, weighted random sampling of a predetermined portion of the corresponding plurality of nucleic acid fragments contributing to the corresponding values of the plurality of genotypic characteristics, wherein the probability of selecting a respective nucleic acid fragment that contributes to the value of a corresponding genotypic characteristic is proportional to the abundance of nucleic acid fragments contributing the corresponding genotypic characteristic relative to the total number of nucleic acid fragments contributing to the values of the plurality of genotypic characteristics.

In some embodiments, for the respective second genotypic data construct corresponding to each respective training subject in the at least one respective training subject: the probability sampling selects a respective first portion of the respective second plurality of nucleic acid fragments that contribute to the values for the plurality of genotypic characteristics and a respective second portion of the respective third plurality of nucleic acid fragments that contribute to the values for the plurality of genotypic characteristics; and the magnitude of the respective first portion of nucleic acid fragments and the respective second portion of nucleic acid fragments is determined based on at least (i) the length of time between the first time point and the second time point and (ii) a temporal model for development of the respective state of the disease condition that the spike-in subject is afflicted with, in the set of states of the disease condition.

In some embodiments, for each respective training subject of the at least one respective training subject, the respective second genotypic data construct was formed by: i) mixing together a first amount of the second plurality of nucleic acid fragments from the second biological sample and a second amount of the cell-free nucleic acids from the spike-in biological sample, thereby forming a mixture of cell-free nucleic acids; ii) sequencing nucleic acid fragments from the mixture of cell-free nucleic acids; and iii) determining values for the plurality of genomic characteristics based on the sequencing ii).

In some embodiments, the first amount and the second amount are determined based on at least (i) the length of time between the first time point and the second time point and (ii) a temporal model for development of the respective state of the disease condition that the spike-in subject is afflicted with, in the set of states of the disease condition.

In some embodiments, the temporal model for development of the respective second state of the disease condition is based at least on a personal characteristic of the respective training subject.

In some embodiments, the disease condition is cancer, and the temporal model for development of the respective state of the cancer is based at least on the type of cancer.

In some embodiments, the disease condition is cancer, and the temporal model for development of the respective state of the cancer is based at least on whether the cancer is metastatic or non-metastatic.

In some embodiments, the disease condition is cancer, and the temporal model for development of the respective state of the cancer is separated into stages.

In some embodiments, the temporal classifier is a logistic regression algorithm.

In some embodiments, the temporal classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

In some embodiments, the temporal classifier is a recurrent neural network.

In some embodiments, the method further comprises: C) obtaining a test time series data set, in electronic form, for a test subject, wherein the test time series data set comprises: for each respective time point in a plurality of time points, a corresponding test genotypic data construct comprising values for the plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points; and D) applying the test time series data set to the temporal classifier to thereby determine the state of the disease condition in the test subject.

In some embodiments, the test subject was not previously diagnosed with the disease condition, prior to the applying D).

Another aspect of the present disclosure provides a method for discriminating a disease condition in a test subject, the method comprising: at a computer system comprising at least one processor and a memory storing at least one program for execution by the at least one processor, the at least one program comprising instructions for: A) obtaining a test time series data set, in electronic form, for a test subject, wherein the test time series data set comprises: for each respective time point in a plurality of time points, a corresponding test genotypic data construct comprising values for a plurality of genotypic characteristics of a corresponding plurality of nucleic acid fragments in a corresponding biological sample obtained from the test subject at the respective time point, and for each respective pair of consecutive time points in the plurality of time points, an indication of the length of time between the respective pair of consecutive time points; and B) applying the test time series data set to a classifier trained according to a method of any one of claims 96-138 to thereby determine the state of the disease condition in the test subject, wherein the plurality of test genotypic characteristics comprises the plurality of genotypic characteristics the classifier was trained against.

In some embodiments, each respective biological sample obtained from the test subject is a liquid biological sample.

In some embodiments, the liquid biological sample is a blood sample.

In some embodiments, the nucleic acid fragments in each biological sample obtained from the test subject are cell-free DNA.

Another aspect of the present disclosure provides a method of assessing the performance of a classifier trained to discriminate a disease condition in a test subject, the method comprising: at a computer system comprising at least one processor and a memory storing at least one program for execution by the at least one processor, the at least one program comprising instructions for: A) obtaining a first classifier trained to discriminate a disease condition by evaluating a test genotypic data construct, wherein the test genomic data construct comprises values for a plurality of genotypic characteristics of a corresponding first plurality of nucleic acid fragments in a first corresponding biological sample obtained from the test subject; B) obtaining an augmented assessment data set comprising a plurality of augmented genotypic data constructs, wherein each respective augmented genotypic data construct in the plurality of augmented genotypic data constructs comprises values for the plurality of genotypic characteristics of a corresponding plurality of nucleic acid fragments representative of a corresponding biological sample obtained from a subject having a respective state of the disease condition in a plurality of states of the disease condition, wherein the augmented assessment data set includes respective augmented genotypic data constructs, in the plurality of augmented genotypic data constructs, that are representative of each respective state of the disease condition in the plurality of states of the disease condition; C) independently applying each respective augmented genotypic data construct in the augmented assessment data set to the classifier to generate a disease state classification for each respective augmented genotypic data construct, thereby generating a plurality of disease state classifications; and D) evaluating each respective disease state classification, in the plurality of disease state classifications, as a function of the respective state of the disease condition represented by the corresponding augmented genotypic data construct, thereby assessing the performance of the classifier.

Another aspect of the present disclosure provides a method of assessing the performance of a classifier trained to discriminate a disease condition in a test subject, the method comprising: at a computer system comprising at least one processor and a memory storing at least one program for execution by the at least one processor, the at least one program comprising instructions for: A) obtaining a first classifier trained to discriminate a disease condition by evaluating a test genotypic data construct, wherein the test genomic data construct comprises values for a plurality of genotypic characteristics of a corresponding first plurality of nucleic acid fragments in a first corresponding biological sample obtained from the test subject; B) obtaining an augmented assessment data set comprising a plurality of augmented genotypic data constructs, wherein each respective augmented genotypic data construct in the plurality of augmented genotypic data constructs comprises values for the plurality of genotypic characteristics of a corresponding plurality of nucleic acid fragments representative of a corresponding biological sample obtained from a subject having a respective state of the disease condition in a plurality of states of the disease condition, wherein the augmented assessment data set includes respective augmented genotypic data constructs, in the plurality of augmented genotypic data constructs, that are representative of each respective state of the disease condition in the plurality of states of the disease condition, wherein the augmented assessment data set is obtained by a method according to any of the preceding methods; C) independently applying each respective augmented genotypic data construct in the augmented assessment data set to the classifier to generate a disease state classification for each respective augmented genotypic data construct, thereby generating a plurality of disease state classifications; and D) evaluating each respective disease state classification, in the plurality of disease state classifications, as a function of the respective state of the disease condition represented by the corresponding augmented genotypic data construct, thereby assessing the performance of the classifier.

In some embodiments, the classifier is a logistic regression algorithm.

In some embodiments, the classifier is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

In some embodiments, the temporal classifier is a recurrent neural network.

In some embodiments, the disease condition is cancer.

In some embodiments, each state in the plurality of states of the cancer comprises a sub-range of cell-free DNA tumor fraction, in a range of cell-free DNA tumor fraction spanning at least from a baseline percentage of cell-free DNA tumor fraction that is at least 25% below the level of detection for the classifier to a ceiling percentage of cell-free DNA tumor fraction that is at least 25% above the level of detection for the classifier.

In some embodiments, the disease condition is a cardiovascular disease.

In some embodiments, each state in the plurality of states of the cardiovascular disease comprises a sub-range of cell-free DNA cardiovascular tissue fraction, in a range of cell-free DNA cardiovascular tissue fraction spanning at least from a baseline percentage of cell-free DNA cardiovascular tissue fraction that is at least 25% below the level of detection for the classifier to a ceiling percentage of cell-free DNA cardiovascular tissue fraction that is at least 25% above the level of detection for the classifier.

In some embodiments, the plurality of genotypic characteristics comprises, for each respective genomic location in a plurality of genomic locations, a methylation status of the respective genomic location.

In some embodiments, the plurality of genotypic characteristics comprises, for each respective genomic location in a plurality of genomic locations, support for a variant allele.

In some embodiments, the plurality of genotypic characteristics comprises, for each respective genomic location in a plurality of genomic locations, a relative copy number.

In some embodiments, the plurality of genotypic characteristics comprises at least 5000 genotypic characteristics.

In some embodiments, the plurality of genotypic characteristics comprises at least 50,000 genotypic characteristics.

In some embodiments, for each biological sample, the values for the plurality of genotypic characteristics of the corresponding plurality of nucleic acid fragments are obtained by whole-genome sequencing.

In some embodiments, for each biological sample, the values for the plurality of genotypic characteristics of the corresponding plurality of nucleic acid fragments are obtained by targeted sequencing using a plurality of nucleic acid probes to enrich the nucleic acids in the corresponding biological sample for a plurality of genomic regions.

In some embodiments, for each biological sample, the values for the plurality of genotypic characteristics of the corresponding plurality of nucleic acid fragments are obtained by (i) whole-genome methylation sequencing or (ii) targeted DNA methylation sequencing using a plurality of nucleic acid probes to enrich the nucleic acids in the corresponding biological sample for a plurality of genomic regions.

In some embodiments, each biological sample is a liquid biological sample.

In some embodiments, the liquid biological sample is a blood sample.

In some embodiments, the plurality of nucleic acid fragments in the corresponding biological sample are cell-free DNA.

### VII. Additional Considerations

The foregoing detailed description of embodiments refers to the accompanying drawings, which illustrate specific embodiments of the present disclosure. Other embodiments having different structures and operations do not depart from the scope of the present disclosure. The term “the invention” or the like is used with reference to certain specific examples of the many alternative aspects or embodiments of the applicants' invention set forth in this specification, and neither its use nor its absence is intended to limit the scope of the applicants' invention or the scope of the claims.

Embodiments of the invention may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Any of the steps, operations, or processes described herein as being performed by the analytics system may be performed or implemented with one or more hardware or software modules of the apparatus, alone or in combination with other computing devices. In one embodiment, a software module is implemented with a computer program product comprising a computer-readable medium containing computer program code, which can be executed by a computer processor for performing any or all of the steps, operations, or processes described.

1. A method for training a model for detecting cancer, comprising:
receiving sequencing data for a plurality of training samples, each training sample labeled as one of cancer and non-cancer and each training sample comprising a plurality of anomalous cfDNA fragments;
sampling a first training sample labeled as cancer and a second training sample labeled as non-cancer;
generating a first synthetic training sample by sampling a first subset of anomalous cfDNA fragments from the first training sample and a second subset of anomalous cfDNA fragments from the second training sample, the first synthetic training sample labeled as cancer;
generating a feature vector for each of the training samples including the first synthetic training sample based on the plurality of anomalous cfDNA fragments of each training sample; and
training the model with the feature vectors and the labels of the training samples including the first synthetic training sample, the model configured to generate a cancer prediction for a test sample based on sequencing data of the test sample.

2. The method of claim 1, wherein generating the first synthetic training sample comprises:
for each genomic region of a plurality of genomic regions, sampling anomalous cfDNA fragments from the first training sample overlapping the genomic region at a first sampling probability and sampling anomalous cfDNA fragments from the second training sample overlapping the genomic region at a second sampling probability that is complementary to the first sampling probability.

3. The method of claim 2, wherein the first sampling probability and the second sampling probability are set according to a limit of detection of the trained model.

4. The method of claim 1, further comprising:
sampling a third training sample labeled as non-cancer; and
generating a second synthetic training sample by sampling a third subset of anomalous cfDNA fragments from the first training sample, wherein the third subset is different from the first subset, and a fourth subset of anomalous cfDNA fragments from the third training sample, the second synthetic training sample labeled as cancer; and
generating a second feature vector for the second synthetic training sample based on the plurality of anomalous cfDNA fragments of the second synthetic training sample,
wherein the model is further trained with the second feature vector and the label of the second synthetic training samples.

5. The method of claim 1, further comprising:
sampling a third training sample labeled as cancer and a fourth training sample labeled as non-cancer;
generating a second synthetic training sample by sampling a third subset of anomalous cfDNA fragments from the third training sample and a fourth subset of anomalous cfDNA fragments from the fourth training sample, the second synthetic training sample labeled as cancer; and
generating a second feature vector for the second synthetic training sample based on the plurality of anomalous cfDNA fragments of the second synthetic training sample,
wherein the model is further trained with the second feature vector and the label of the second synthetic training samples.

6. The method of claim 5, wherein the first training sample and the first synthetic training sample have a label of a first cancer type, and wherein the third training sample and the second synthetic training sample have a label of a second cancer type.

7. The method of claim 1, wherein each feature of a feature vector corresponds to a CpG site of a plurality of CpG sites, and wherein generating a feature vector for each of the training samples comprises:
for each anomalous cfDNA fragment, determining a likelihood that the anomalous cfDNA fragment is derived from a cancer biological sample by applying a probabilistic model to a plurality of methylation states at a plurality of CpG sites of the anomalous cfDNA fragment; and
determining each feature of the feature vector according to a count of anomalous cfDNA fragments overlapping the CpG site corresponding to the feature and having a likelihood above a threshold likelihood.

8. The method of claim 7, wherein each feature vector is normalized according to a sequencing depth of the training sample.

9. The method of claim 1, further comprising:
filtering an initial set of cfDNA fragments for each training sample with p-value filtering to generate the set of anomalous fragments, the filtering comprising removing fragments from the initial set having below a threshold p-value with respect to other fragments to produce the set of anomalous fragments.

10. The method of claim 1, wherein the trained model is a neural network algorithm, a support vector machine algorithm, a Naive Bayes algorithm, a nearest neighbor algorithm, a boosted trees algorithm, a random forest algorithm, a decision tree algorithm, a multinomial logistic regression algorithm, a linear model, or a linear regression algorithm.

11. (canceled)

12. A method for detecting cancer, comprising:
receiving sequencing data for a test sample comprising a plurality of anomalous cfDNA fragments;
generating a test feature vector based on the anomalous cfDNA fragments of the test sample; and
inputting the test feature vector into a classification model to generate a cancer prediction for the test sample, wherein the classification model is trained by:
receiving sequencing data for a plurality of training samples, each training sample labeled as one of cancer and non-cancer and each training sample comprising a plurality of anomalous cfDNA fragments,
sampling a first training sample labeled as cancer and a second training sample labeled as non-cancer,
generating a first synthetic training sample by sampling a first subset of anomalous cfDNA fragments from the first training sample and a second subset of anomalous cfDNA fragments from the second training sample, the first synthetic training sample labeled as cancer,
generating a feature vector for each of the training samples including the first synthetic training sample based on the plurality of anomalous cfDNA fragments of each training sample, and
training the model with the feature vectors and the labels of the training samples including the first synthetic training sample.

13. The method of claim 12, wherein the cancer prediction is a binary prediction between cancer and non-cancer.

14. The method of claim 12, wherein the cancer prediction is a multiclass cancer prediction between a plurality of cancer types.

15. The method of claim 12, wherein each feature of a feature vector corresponds to a CpG site of a plurality of CpG sites, and wherein generating a feature vector for each of the training samples comprises:
for each anomalous cfDNA fragment, determining a likelihood that the anomalous cfDNA fragment is derived from a cancer biological sample by applying a probabilistic model to a plurality of methylation states at a plurality of CpG sites of the anomalous cfDNA fragment; and
determining each feature of the feature vector according to a count of anomalous cfDNA fragments overlapping the CpG site corresponding to the feature and having a likelihood above a threshold likelihood.

16. The method of claim 15, wherein each feature vector is normalized according to a sequencing depth of the training sample.

17. The method of claim 12, wherein the classification model is trained by further:
filtering an initial set of cfDNA fragments for each training sample with p-value filtering to generate the set of anomalous fragments, the filtering comprising removing fragments from the initial set having below a threshold p-value with respect to other fragments to produce the set of anomalous fragments.

18. (canceled)

19. A system comprising:
a computer processor; and
a non-transitory computer-readable storage medium storing instructions that, when executed by the computer processor, cause the processor to perform operations comprising:
receiving sequencing data for a test sample comprising a plurality of anomalous cfDNA fragments;
generating a test feature vector based on the anomalous cfDNA fragments of the test sample; and
inputting the test feature vector into a classification model to generate a cancer prediction for the test sample, wherein the classification model is trained by:
receiving sequencing data for a plurality of training samples, each training sample labeled as one of cancer and non-cancer and each training sample comprising a plurality of anomalous cfDNA fragments,
sampling a first training sample labeled as cancer and a second training sample labeled as non-cancer,
generating a first synthetic training sample by sampling a first subset of anomalous cfDNA fragments from the first training sample and a second subset of anomalous cfDNA fragments from the second training sample, the first synthetic training sample labeled as cancer,
generating a feature vector for each of the training samples including the first synthetic training sample based on the plurality of anomalous cfDNA fragments of each training sample, and
training the model with the feature vectors and the labels of the training samples including the first synthetic training sample.

20. The system of claim 19, wherein the cancer prediction is at least one of:
a binary prediction between cancer and non-cancer; and
a multiclass cancer prediction between a plurality of cancer types.

21. The system of claim 19, wherein each feature of a feature vector corresponds to a CpG site of a plurality of CpG sites, and wherein generating a feature vector for each of the training samples comprises:
for each anomalous cfDNA fragment, determining a likelihood that the anomalous cfDNA fragment is derived from a cancer biological sample by applying a probabilistic model to a plurality of methylation states at a plurality of CpG sites of the anomalous cfDNA fragment; and
determining each feature of the feature vector according to a count of anomalous cfDNA fragments overlapping the CpG site corresponding to the feature and having a likelihood above a threshold likelihood.

22. The system of claim 19, wherein the classification model is trained by further:
filtering an initial set of cfDNA fragments for each training sample with p-value filtering to generate the set of anomalous fragments, the filtering comprising removing fragments from the initial set having below a threshold p-value with respect to other fragments to produce the set of anomalous fragments.
